(19) United States
(12) Patent Application Publication
(10) Pub. No.: US 2021/0260009 A1
(43) Pub. Date: Aug. 26, 2021

(54) METHODS AND COMPOSITIONS FOR THE TREATMENT OF A RENAL DISEASE

(71) Applicant: PRESIDENT AND FELLOWS OF HARVARD COLLEGE, CAMBRIDGE, MA (US)

(72) Inventor: Wendy GARRETT, Boston, MA (US)

(73) Assignee: PRESIDENT AND FELLOWS OF HARVARD COLLEGE, CAMBRIDGE, MA (US)

(21) Appl. No.: 17/180,136

(22) Filed: Feb. 19, 2021

Related U.S. Application Data
(60) Provisional application No. 62/979,638, filed on Feb. 21, 2020.

(51) Int. Cl. A61K 31/198; A61P 13/12; A61K 9/00; A23L 33/00; A23L 33/175; A23K 20/142; A23K 50/40

(52) U.S. Cl. CPC A61K 31/198; A61P 13/12; A61K 9/0056; A61K 9/0053; A23V 2002/00; A23L 33/40; A23L 33/175; A23K 20/142; A23K 50/40; A61K 9/0029

(57) ABSTRACT

Provided herein are methods, compositions, and assays related to regulating the level or activity of hydrogen sulfide (H₂S) in a subject. The methods, compositions, and assays are also related to treating, alleviating, and preventing an inflammatory or fibrotic disease of the kidney in a subject in need thereof.

### CROSS-REFERENCE TO RELATED APPLICATION

This application claims benefit under 35 U.S.C. § 119(e) of U.S. Provisional Application No. 62/979,638 filed Feb. 21, 2020, the content of which is incorporated herein by reference in its entirety.

### GOVERNMENT SUPPORT

This invention was made with government support under Grant No. R01 CA202704, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 19, 2021, is named 002806-096980USPT_SL.txt and is 69,354 bytes in size.

### TECHNICAL FIELD

The technology described herein relates to methods, compositions, and assays for regulating hydrogen sulfide and the treating a renal disease.

### BACKGROUND

The gut microbiota produces a myriad of diet-derived microbial metabolites that function in microbe-microbe and host-microbe interactions. Furthermore, microbial flora can influence the development and severity of a multitude of diseases. Associations between chronic kidney disease (CKD) and the gut microbiota have been postulated, yet questions remain about the underlying mechanisms. In humans, increasing dietary protein increases gut bacterial production of indole, an indoxyl sulfate precursor, and hydrogen sulfide (H₂S). H₂S has diverse physiological functions, some of which are mediated by the post-translational modification S-sulfhydration. The physiological roles of H₂S in regulating gut bacterial function within a host remain understudied. Furthermore, there is currently an unmet need for dietary and pharmaceutical compositions that can inhibit tryptophanase activity in bacteria and regulate H₂S in in the gut for the treatment and prevention of renal diseases.

### SUMMARY

The methods, compositions, and assays provided herein are based, in part, on the discovery that a sulfur amino acid-based dietary intervention post-translationally modifies a microbial enzyme, tryptophanase (TnaA), blunting its uremic toxin-producing activity and alleviating CKD in a preclinical model. Furthermore, it was discovered that gut microbiota influences the severity of renal diseases, particularly inflammatory and fibrotic diseases of the kidney.

In one aspect, provided herein is a method of regulating the level or activity of hydrogen sulfide (H₂S) in the gastrointestinal tract of a subject, the method comprising: administering to the subject an agent that increases the level or activity of H₂S, e.g., in the gastrointestinal tract of the subject as compared to a reference level.

In another aspect, provided herein is a method of treating an inflammatory or fibrotic disease of the kidney in a subject, the method comprising: administering to a subject in need thereof an agent that increases the level or activity of H₂S, e.g., in the gastrointestinal tract of the subject as compared to a reference level.

In yet another aspect, provided herein is a method of preventing or alleviating an inflammatory or fibrotic disease of the kidney in a subject, the method comprising: administering to a subject in need thereof an agent that increases the level or activity of H₂S, e.g., in the gastrointestinal tract of the subject as compared to a reference level.

In some embodiments of any one of the aspects described herein, the agent that increases the level or activity of H₂S is a TnaA inhibitor.

In some embodiments of any one of the aspects described herein, the agent that increases the level or activity of H₂S can reduce gut indole levels as compared with a reference level.

In some embodiments of any one of the aspects described herein, the agent that increases the level or activity of H₂S is a sulfur donor, e.g., the thiol group of a reactive Cys is modified to a persulfide (—SSH) group.

In some embodiments of any one of the aspects described herein, the agent or composition modulates the level or activity of a bacterial cysteine desulfhydrase polypeptide. In some embodiments of any one of the aspects described herein, the agent or composition modulates the level of a bacterium expressing a cysteine desulfhydrase polypeptide.

In some embodiments of any one of the aspects described herein, the agent or composition modulates or increases the level or activity of a polypeptide or a nucleic acid encoding a polypeptide selected from the group consisting of: cystathionine β-synthase; cystathionine γ-lyase, 3-mercaptopyruvate sulfurtransferase; and cysteine aminotransferase.

In some embodiments of any one of the aspects described herein, the agent that increases the level or activity of H₂S is a sulfated amino acid. Some exemplary sulfated amino acids include, but are not limited to, methionine, cysteine, homocysteine, taurine, cystine or di-cysteine, and salts, analogs, and derivatives thereof.

The agent that increases the level or activity of H₂S can be comprised in a composition. For example, the agent that increases the level or activity of H₂S can be comprised in a food composition. The food composition can be for consumption by a mammal, for example by human or a pet. In some embodiments of any one of the aspects described herein, the composition can be formulated as a dietary supplement. In some embodiments of any of the aspects described herein, the composition can be formulated as a medical food. For example, the composition can be formulated as a medical pet food.

In another example, the agent that increases the level or activity of H₂S can be comprised in a pharmaceutical composition, e.g., a composition comprising the agent and a pharmaceutically acceptable carrier or excipient.

It is noted that the agent that increases the level or activity of H₂S can be administered in any suitable route of administration. For example, the agent can be administered orally, enterally or parenterally. Accordingly, in some embodiments of any one of the aspects, the agent can be formulated in a composition for oral administration, enteral administration, or parenteral administration.

In one aspect, provided herein is a method of regulating the level or activity of hydrogen sulfide (H₂S) in the gastrointestinal tract of a subject, the method comprising: administering to the subject a composition comprising a sulfated amino acid.

In another aspect, provided herein is a method of regulating the level or activity of hydrogen sulfide (H₂S) in the gastrointestinal tract of a subject, the method comprising: administering to the subject a food composition comprising a sulfated amino acid.

In another aspect, provided herein is a method of regulating the level or activity of hydrogen sulfide (H₂S) in the gastrointestinal tract of a subject, the method comprising: administering to the subject a pharmaceutical composition comprising a sulfated amino acid.

In another aspect, provided herein is a method of treating an inflammatory or fibrotic disease of the kidney in a subject, the method comprising: administering to a subject in need thereof a composition comprising a sulfated amino acid.

In another aspect, provided herein is a method of treating an inflammatory or fibrotic disease of the kidney in a subject, the method comprising: orally administering to a subject in need thereof a composition comprising a sulfated amino acid.

In another aspect, provided herein is a method of preventing or alleviating an inflammatory or fibrotic disease of the kidney in a subject, the method comprising: orally administering to a subject in need thereof a composition comprising a sulfated amino acid.

In another aspect, provided herein is an assay for identifying an agent for the treatment of an inflammatory or fibrotic disease of the kidney in a subject, the assay comprising:

a. contacting a bacterium with an agent; and

b. detecting the level or activity of hydrogen sulfide (H₂S).

In some embodiments of any one of the aspects, the agent that increases the level or activity of H₂S is selected for the treatment of an inflammatory or fibrotic disease of the kidney in a subject.

In another aspect, provided herein is a composition comprising:
a. an effective amount of a sulfated amino acid, i.e., an amount that increases the level or activity of H₂S in a subject; and
b. a carrier.

In some embodiments of any one of the aspects, the sulfated amino acid is isolated and purified.

In some embodiments of any of the aspects, the composition is formulated as a dietary supplement. In some embodiments of any of the aspects, the composition is formulated as a medical food. In some embodiments of any of the aspects, the composition is formulated as a medical pet food. In some embodiments of any of the aspects, the composition is formulated as a pharmaceutical composition.

In some embodiments of any of the aspects, the composition comprises at least one food ingredient. In some embodiments of any of the aspect, the carrier is a food ingredient. In some embodiments of any of the aspects, the food ingredient is selected from the group consisting of: fats, carbohydrates, proteins, fibers, nutritional balancing agents, and mixtures thereof. In some embodiments of any of the aspects, food ingredient is selected from the group consisting of: fats, carbohydrates, proteins, fibers, nutritional balancing agents, and mixtures thereof. In some embodiments of any of the aspects, the composition further comprises adenine, one or more vitamins, potassium, omega 3-fatty acids, and/or calcium carbonate. In some embodiments of any of the aspects, the composition is a cat food or a dog food formulated to enhance or improve renal function.

In some embodiments of any of the aspects, the administering is oral administration, enteral administration, or parenteral administration.

In some embodiments of any of the aspects, the subject is a mammal. In some embodiments of any of the aspects, the subject is a human, a dog, or a cat. In some embodiments of any of the aspects, the subject has or is suspected of having an inflammatory or fibrotic disease of the kidney.

In some embodiments of any of the aspects, the inflammatory or fibrotic disease of the kidney is selected from the group consisting of: chronic kidney disease, renal parenchymal injury, tubulitis, end-stage renal failure, lupus, nephritis, acute renal failure, kidney infection, polycystic kidney disease, renal amyloidosis, and renal colic. In some embodiments of any of the aspects, the subject has or is suspected of having an enrichment of one or more bacteria selected from the group consisting of: Enterobacteriaceae, Escherichia, Escherichia coli, Bacterioides, Prevotella, Ordoribacter, Cuhuromica, Alistipes, Pseudoflavonifractor, Pseudoflavonifractor sp. Marseille-P3106, Alistipes putredinis, Bacteroides intestinalis, Bacteroides thetaiotaomicron, Bacteroides acidifaciens, Bacteroides uniformis, Bacteroides nordii, Bacteroides clarus, Prevotella sp. CAG 1031, Bacteroides sp. CAG 462, Ordoribacter splanchnicus, Cuhuromica massihensis, Alistipes sp. An66, and Alistipes sp. CHKCI003 in the gastrointestinal tract.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIGS. 1A-1F shows Dietary Saa and the Gut Microbiota Modulate Kidney Injury Severity in a Mouse CKD Model. FIG. 1A shows serum creatinine levels of SPF and GF C57BL/6J mice on low vs. high Saa+Ade diets. Symbols indicate data from individual mice. FIG. 1B shows representative H&E staining of kidneys from mice in FIG. 1A. FIG. 1C shows representative trichrome stain of kidneys from mice in FIG. 1A. FIG. 1D shows histology-based renal injury score. Data represent 3 independent experiments and symbols indicate data from individual mice. FIGS. 1E and 1F show cecal sulfide levels using lead acetate (FIG. 1E) or methylene blue sulfide detection assays (FIG. 1F) from SPF and GF C57BL/6J mice on low vs. high Saa diets. Data represent 3-4 independent experiments and symbols indicate data from individual mice. Bars represent the mean±SEM. ** P value <0.01, *** P value <0.001. Two-way ANOVA with Tukey's post-hoc test for FIGS. 1A, 1D, 1E and 1F.

FIGS. 2A-2E shows cecal microbiome 16S rRNA gene amplicon analysis of mice on Saa diets. FIG. 2A shows alpha-diversity measures. FIG. 2B shows beta-diversity, weighted UniFrac analysis. FIG. 2C shows relative abundances of bacterial phyla. FIG. 2D shows relative abundances of the top 10 bacterial genera. FIG. 2E shows the same analysis as in FIG. 2D, each X-axis tick represents a cage, allowing for observation of caging effects. n=19 mice on low Saa diet and 24 mice on high Saa diet.

FIGS. 3A-3E shows CKD Patient Fecal Microbiome Data Analysis Reveals Enterobacteriaceae Enrichment. FIG. 3A shows LEfSe analysis of 16S rRNA gene amplicon survey data from Kai-Yu et al. (2017). FIG. 3B shows LEfSe analysis of 16S rRNA gene amplicon survey data from an unpublished CKD patient cohort (NCBI accession PRJEB5761). For clarity, taxonomy is shown from the class level. FIG. 3C shows Volcano plot of PhyloChip analysis data from Vaziri et al. (2013). Taxa with fold change >2 and q-value <0.05 are labeled in color with their family level taxonomy. FIG. 3D shows boxplot representation of the combined averaged relative abundance of 7 E. coli strains measured in the fecal samples from CKD and non-CKD subjects using PhyloChip analysis. FIG. 3E shows boxplot representation of normalized mean E. coli gene abundance in a whole genome shotgun dataset of CKD versus non-CKD subjects (NCBI accession PRJNA449784). * P value <0.05, ** P value <0.01.

FIG. 4A shows serum creatinine levels from WT ASF^{E. coli} mice on low vs. high Saa diets. Data represent 2-3 independent experiments and symbols indicate data from individual mice. FIG. 4B shows colonization of ASF mice with E. coli on Saa+Ade diets. Data represent 2-3 independent experiments and symbols indicate data from individual mice. FIG. 4C shows colonization of ASF mice with E. coli on Saa diets. Data represent 2-3 independent experiments and symbols indicate data from individual mice. FIG. 4D shows relative abundances of ASF strains in cecal contents of mice on Saa+Ade diets. Data represent 2-3 independent experiments and symbols indicate data from individual mice. Bars represent the mean±SEM. * P value <0.05. Mann-Whitney U test for A.

FIGS. 5A-5F show E. coli Colonization of ASF Mice Exacerbates Kidney Injury in a Diet-dependent Manner in a Mouse Model of CKD. FIG. 5A shows serum creatinine levels from ASF or ASF E. coli C57BL/6J mice on low vs. high Saa+Ade diets. Data represent 2-3 independent experiments and symbols indicate data from individual mice. FIG. 5B shows representative H&E staining of kidneys from mice in A. FIG. 5C shows representative trichrome staining of kidneys from mice in A. D. Histology-based renal injury score. Data represent 2-3 independent experiments and symbols indicate data from individual mice. FIG. 5E-5F shows measurement of cecal sulfide levels using the lead acetate paper (FIG. 5E) or methylene blue assays (FIG. 5F) on cecal contents from ASF or ASF E. coli C57BL/6J mice on low vs high Saa diets. Data represent 2-3 independent experiments and symbols indicate data from individual mice. Bars represent the mean±SEM. * P value <0.05, ** P value <0.01. Two-way ANOVA with Tukey's post-hoc test for FIGS. 5A and 5D, and Mann-Whitney test for FIGS. 5E and 5F.

FIGS. 6A-6H show characterization of E. coli S-Sulfhydrome Reveals that TnaA is a Highly S-Sulfhydrated Protein. FIG. 6A shows E. coli production of sulfide from L-cysteine, during aerobic growth in LB broth, detected by lead acetate sulfide assay. Representative data showing reduced sulfide production by the ΔdecR mutant (left) and quantitative densitometry (right). Data represent 4 independent experiments. FIG. 6B shows measurement of sulfide produced by E. coli cultures by methylene blue sulfide assay. Data represent 6 independent experiments. FIG. 6C shows schematic of the S-sulfhydrated protein pull-down method. FIG. 6D shows Silver staining of E. coli lysates subjected to S-sulfhydration pull-down and eluted either with or without 20 mM DTT, data shown represent 3 independent experiments. FIG. 6E shows Silver staining of WT and ΔdecR E. coli lysates subjected to S-sulfhydration pull-down and eluted with DTT, data shown represent 2 independent experiments. FIG. 6F shows (L) Heat map of the relative quantity of the 212 S-sulfhydrated proteins identified and quantified by TMT LC-MS3 analysis in the S-sulfhydration pull-down fractions from WT E. coli samples eluted with or without DTT and from ΔdecR mutant samples eluted with DTT. Proteins are ordered based on their q-value score for enrichment in the DTT eluted vs non-DTT eluted samples. Data represent 3 biological repeats and are normalized to the reference channel, comprised of equal amounts from each of the 9 samples. (right) Top 10 S-sulfhydrated proteins ranked by q-value score. FIG. 6G shows boxplot representation of the data presented in F. FIG. 6H shows pathway enrichment analysis using the PANTHER database (Mi et al., 2019) of the 212 S-sulfhydrated proteins. Pathways with q-value <0.05 are reported. Bars represent the mean±SEM. ** P value <0.01, *** P value <0.001. Linear model test for FIG. 6A, two-way Kruskal-Wallis test with Dunn's post-hoc test for B and Two-way ANOVA with Tukey's post-hoc test for FIG. 6G.

FIG. 7A shows final OD600 of WT and ΔdecR E. coli cultures grown in LB supplemented with cysteine under aerobic conditions. Data represent 3 independent experiments. FIG. 7B shows lead acetate detection of H₂S production by WT and ΔdecR E. coli cultures grown in LB supplemented with cysteine under anaerobic conditions. Data represent 3 independent experiments. FIG. 7C shows final OD600 of WT and ΔdecR E. coli cultures grown in LB supplemented with cysteine under anaerobic conditions. Data represent 3 independent experiments. FIG. 7D shows Coomassie staining of S-sulfhydrated proteins from WT E. coli lysates treated with NaCl, H₂O₂ or NaHS. Lower gel shows Western blotting of RpoD in the flow-through samples, as loading control. Data are representative of 3 independent experiments. FIG. 7E shows Coomassie staining of S-sulfhydrated proteins from WT E. coli grown in LB or LB supplemented with 0.4 mM cysteine. Lower gel shows Western blotting of RpoD in the flow-through samples, as loading control. FIG. 7F shows quantification of several Coomassie stains from E. Data are representative of 3 independent experiments. Bars represent the mean±SEM. ** P value <0.01. Mann-Whitney U test for FIG. 7E.

FIG. 8A-8F show Indole-Producing Enzymatic Activity of E. coli TnaA is Inhibited by S-Sulfhydration. FIG. 8A shows representative western blot analysis of TnaA-His from WT and ΔdecR E. coli lysates subjected to S-sulfhydration pull-down. The loading control shows the quantity of RpoD, a housekeeping protein, in the flow-through samples. Data represent 3 independent experiments. The graph below the blot represents quantification of the ratio of densitometry between TnaA in pull-down samples and RpoD in flow-through samples. FIG. 8B shows the same analysis method as in A depicting the effects of treatments of E. coli lysates with NaCl, H2O2, or NaHS on TnaA S-sulfhydration. Data represent 4 independent experiments. FIG. 8C shows LC-MS/MS m/z spectra of a TnaA S-sulfhydrated peptide. The top spectrum shows the addition of +32 Da on C363, red series ions represent the native cysteine residue (R—SH) peptide and the blue series ions represent the R—SO2/R—S—SH cysteine modification. The lower spectrum represents the addition of +64 Da on C363, red series ions represent the native cystine residue (R—SH) and the blue series ions represent the oxidized S-sulfhydrated (R—S—SO2) or the polysulfhydrated (R—S—S—S) cysteine residue. FIG. discloses SEQ ID NO: 9. FIG. 8D shows LC-MS/MS analysis of the relative quantity of indoles in bacterial cultures of WT E. coli supplemented with cysteine or NaHS. Data represent 5 independent experiments. FIG. 8E shows Kovac's assay for indole concentrations in bacterial cultures of WT E. coli supplemented with cysteine or NaHS. Data represent 4 independent experiments. FIG. 8F shows Kovac's assay for indole production by purified TnaA enzyme in buffer supplemented with NaCl, Na2S4, cysteine or DTT. Data represent 3 independent experiments. Bars represent the mean±SEM. * P value <0.05, ** P value <0.01, *** P value <0.001. Mann-Whitney test for FIG. 8A, and Two-way ANOVA with Tukey's post-hoc test for FIGS. 8B and 8F, and two-way Kruskal-Wallis test with Dunn's post-hoc test for FIGS. 8D and 8E.

FIG. 9A shows relative indole levels of WT and ΔdecR E. coli cultures grown in LB supplemented with cysteine measured by LC-MS/MS. Data represent 3 independent experiments. FIG. 9B shows Western blotting of TnaA in WT E. coli cultures grown in LB or LB supplemented with cysteine or NaHS, or ΔdecR E. coli grown in LB. Lower gel shows Western blotting for RpoD, as loading control. Data represent 2-3 independent experiments. FIG. 9C shows relative indole levels of WT and tnaA mut E. coli cultures grown in LB measured by Kovac's assay. Data represent 3 independent experiments. FIG. 9D shows Western blotting of the S-sulfhydration pull-down fractions of purified E. coli TnaA treated with NaCl, NaHS or Na2S4. Flow through samples represent loading control. Gels are representative of 3 independent experiments. FIG. 9E shows normalized activity of TnaA purified from WT and ΔdecR E. coli grown with 5 mM L-cysteine. Activity was normalized to protein concentration. Bars represent the mean±SEM. ** P value <0.01. Mann-Whitney U test for FIG. 9A.

FIG. 10A shows colonization of ASF mice with E. coli on Saa diets. Data represent 3 independent experiments and symbols indicate data from individual mice. FIG. 10B shows a chromatogram of indole detection in cecal contents from ASF mice on Saa diets. Data represent 3 independent experiments and symbols indicate data from individual mice. FIG. 10C shows colonization of ASF mice with different E. coli strains on low Saa+Ade diet. Data represent 2-3 independent experiments and each symbol represents data from an individual mouse. FIG. 10D shows colonization of ASF mice with different E. coli strains on high Saa+Ade diet. Data represent 2-3 independent experiments and each symbol represents data from an individual mouse. FIG. 10E shows LC-MS measurements of serum indoxyl sulfate in ASF mice on high Saa+Ade diet, colonized with WT, tnaA mut or ΔdecR E. coli strains. Data represent 2-3 independent experiments and each symbol represents data from an individual mouse. FIG. 10F shows serum creatinine levels of mice in FIG. 10E. Data represent 2-3 independent experiments and each symbol represents data from an individual mouse. FIG. 10G shows representative H&E staining of kidneys from mice in FIG. 10E. FIG. 10H shows representative trichrome staining of kidneys from mice in E. FIG. 10I shows histology-based renal injury score of mice in FIG. 10E. Data represent 2-3 independent experiments and symbols indicate data from individual mice. Bars represent the mean±SEM. * P value <0.05 Two-way ANOVA with Tukey's post-hoc test for FIG. 10F.

FIG. 11A-11I demonstrates that Dietary Saa Modulate Cecal Indole Levels, Serum Indoxyl-Sulfate Levels, and Kidney Function in a Mouse CKD Model. FIG. 11A shows Western blot analysis of TnaA of S-sulfhydration pull-down fraction and flow-through samples from cecal contents of ASF E. coli mice on Saa diets. The data are representative of pooled samples from 3 mice per condition from 3 independent experiments. FIG. 11B shows Kovac's assay measurement of indole levels in cecal contents from ASF E. coli mice on Saa diets. Data represent 3 independent experiments and each symbol represents data from an individual mouse. FIG. 11C shows LC-MS/MS analysis of indole levels in cecal contents from ASF E. coli mice on Saa diets. Left, spectra representative of an experiment with 3 mice in each group and the indole standard used. Right, quantification of the LC-MS/MS analysis. Data represents 3 independent experiments and each symbol represents data from an individual mouse. FIG. 11D shows LC-MS measurements of serum indoxyl sulfate in ASF and ASF mice on low Saa+Ade diet, colonized with WT, tnaA mut or ΔdecR E. coli strains. Data represent 2-3 independent experiments and each symbol represents data from an individual mouse. FIG. 11E shows serum creatinine levels in ASF and ASF^{E.coli} mice colonized with WT, tnaA mut or ΔdecR E. coli strains on low Saa+Ade diets. Data represent 2-3 independent experiments and each symbol represents data from an individual mouse. FIG. 11F shows representative H&E staining of kidneys from mice in E. FIG. 11G shows representative trichrome staining of kidneys from mice in FIG. 11E. FIG. 11H shows histology-based renal injury score. Data represent 2-3 independent experiments and symbols indicate data from individual mice. FIG. 11I shows schematic representation of the effects of low and high Saa-Ade diets on gut microbial activity and the consequences for renal function. Bars represent the mean±SEM. * P value <0.05, ** P value <0.01, *** P value <0.001. Mann-Whitney test for FIGS. 11A, 11B and 11C. two-way ANOVA with Tukey's post-hoc test for FIGS. 11D, 11E and 11H.

FIG. 12 shows multiple sequence alignment of TnaA from several human gut microbiota bacterial species. FIG. discloses SEQ ID NOS 10-25, respectively, in order of appearance.

FIG. 13 shows modifications and S-sulfhydration of TnaA. FIG. discloses SEQ ID NO: 26.

FIG. 14A shows colonization of ASF mice with E. coli on Saa+Ade diets. FIG. 14B shows Colonization of ASF mice with E. coli on Saa diets. FIG. 14C shows relative abundances of ASF strains in cecal contents of mice on Saa+Ade diets. FIG. 14D shows RT-qPCR analysis of CKD related genes from kidneys of GF and SPF mice on Saa+Ade diets. FIGS. 14E and 14F show serum Cre levels from WT ASF^{E.} coli (E) and WT SPF (F) mice on low vs. high Saa diets. Data represent 2 independent experiments for FIGS. 14D and 14F, and 3 independent experiments for A, B, C, and E. Symbols represent individual mice. Bars represent mean±SEM. * P value <0.05, ** P value <0.01, *** P value <0.001. Symbols represent individual mice. Bars represent mean±SEM. Two-way ANOVA with Tukey's post-hoc test for D. Mann-Whitney test for FIG. 14E and FIG. 14F.

FIG. 15A shows relative indole levels of WT and ΔdecR E. coli cultures grown aerobically in LB supplemented with cysteine measured by LC-MS/MS. FIG. 15B shows Western blotting of TnaA in WT E. coli cultures grown aerobically in LB or LB supplemented with cysteine or NaHS, or ΔdecR E. coli grown in LB. Lower gel shows Western blotting for RpoD as the loading control. FIG. 15C shows relative indole levels of WT and tnaA mut E. coli cultures grown aerobically in LB measured by Kovac's assay. FIG. 15D shows Western blotting of the S-sulfhydration pull-down fractions of purified E. coli TnaA treated with NaCl, H₂O₂ or Na2S4. Flow through samples represent loading control. FIG. 15E shows Normalized activity of TnaA purified from WT and ΔdecR E. coli grown with 5 mM L-cysteine. Activity was normalized to protein concentration. Data represent 3 independent experiments for FIGS. 15A, 15B, 15C, 15D and 15E. Bars represent mean±SEM. * P value <0.05, ** P value <0.01. Mann-Whitney test for FIGS. 15A, 15C and 15E.

FIG. 16 shows additional growth curves of WT and ΔdecR E. coli in LB medium supplemented with various L-cysteine concentrations under aerobic conditions. (Right) Lead acetate detection of H₂S production by WT and ΔsseA E. coli cultures grown in LB supplemented with cysteine under aerobic conditions. See also FIGS. 7A-7F.

### DETAILED DESCRIPTION

The methods, compositions, and assays described herein are based, in part, on the discovery that hydrogen sulfide (H₂S) and/or S-sulfhydration in the gastrointestinal tract of a subject can affect renal function via dietary cysteine, which can affect the level and activity of hydrogen sulfide (H₂S) and S-sulfhydration in the gut. Specifically, it was discovered that the combination of a low sulfated amino acid (Saa) diet and the presence of a bacterium (e.g., E. coli, an Enterobacteriaceae family member), can markedly exacerbate kidney damage in a model of renal disease (e.g., chronic kidney disease, CDK). Therefore, increasing the levels of H₂S, inhibit bacterial enzyme function, such as microbial enzyme-tryptophanase (TnaA) blunts the microbial enzyme's uremic toxin-producing activity and alleviates CKD.

Briefly, the methods, compositions, and assays provided herein relate to regulating the levels of hydrogen sulfide (H₂S) and/or S-sulfhydration in the gastrointestinal tract of a subject. The methods compositions, and assays provided herein also relate to treating, alleviating, and preventing a renal disease. In particular, the renal disease is an inflammatory and/or fibrotic disease of the kidney.

In one aspect, provided herein is a method of regulating the level or activity of hydrogen sulfide (H₂S) in the gastrointestinal tract of a subject, the method comprises: administering to the subject a composition comprising a sulfated amino acid.

Hydrogen sulfide (H₂S) is a highly reactive molecule that also functions as a signaling compound in the body. H₂S has diverse physiological functions, some of which are mediating post-translational modification of sulfated amino acids (e.g., cysteine, Cys), a process called S-sulfhydration. Non-limiting examples of S-sulfhydration can be found, e.g., in Paul and Snyder, Nat Rev Mol Cell Biol (2012); Linden, 2014; Magee et al., 2000; and Mustafa et al., 2009; the contents of each of which are incorporated herein by reference in their entireties. Specifically, in the process of sulfhydration, the thiol group of a reactive Cys is modified to a persulfide (—SSH) group, resulting in increased reactivity of the Cys residue.

The methods provided herein are methods of regulating or modulating the levels and activity of H₂S in a subject. In some embodiments of any one of the aspects, the method comprises administering an agent or composition that increases the level or activity of H₂S in the gastrointestinal tract of a subject compared to a reference level. The increased level or activity of H₂S can increase S-sulfhydration of microbial enzymes (e.g., tryptophanase, TnaA) which affects enzymatic function. For example, tryptophanase (TnaA), an exo-enzyme that degrades tryptophan to ammonia, pyruvate and indole is inhibited by S-sulfhydration, and regulates the levels of indoxyl sulfate in the body (e.g, serum). Furthermore, the mechanism by which S-sulfhydration of TnaA can influence renal function is provided in the working examples.

Generally, tryptophanase or TnA is a microbial enzyme that catalyzes the degradation of tryptophan to indole, pyruvate and ammonia. Indoles are a class of bacterial-produced molecules that not only regulate bacterial physiology, but also participate in bacteria-host interactions. Indoles can be transported through the portal vein to the liver where they are oxidized, yielding the uremic toxin indoxyl sulfate. Indole are further described in the art, e.g., in Darkoh, K. et al. “Clostridium difficile Modulates the Gut Microbiota by Inducing the Production of Indole, an Interkingdom Signaling and Antimicrobial Molecule.” mSystems. 4, e00346-18, /msystems/4/2/msys.00346-18.atom (2019); and T. Zelante, et al. Tryptophan catabolites from microbiota engage aryl hydrocarbon receptor and balance mucosal reactivity via interleukin-22. Immunity. 39, 372-385 (2013).

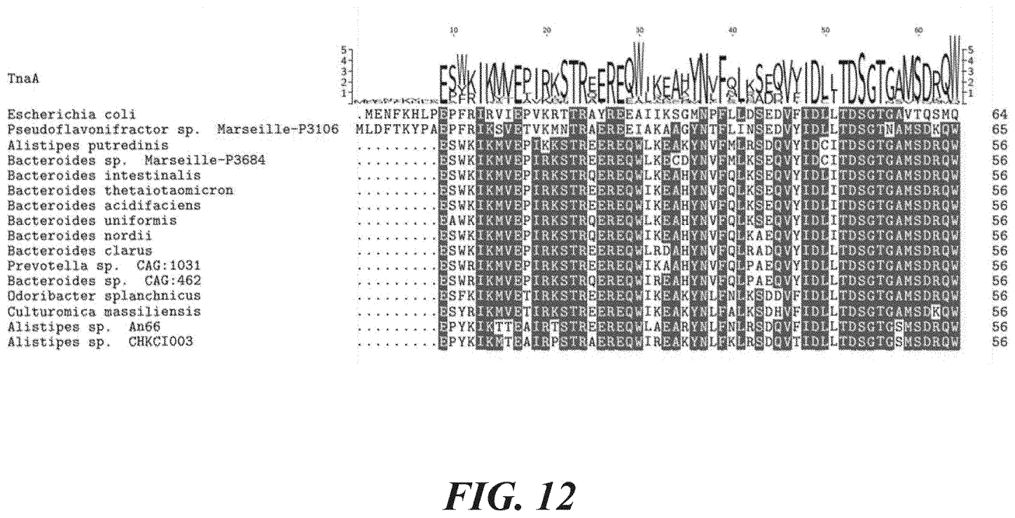

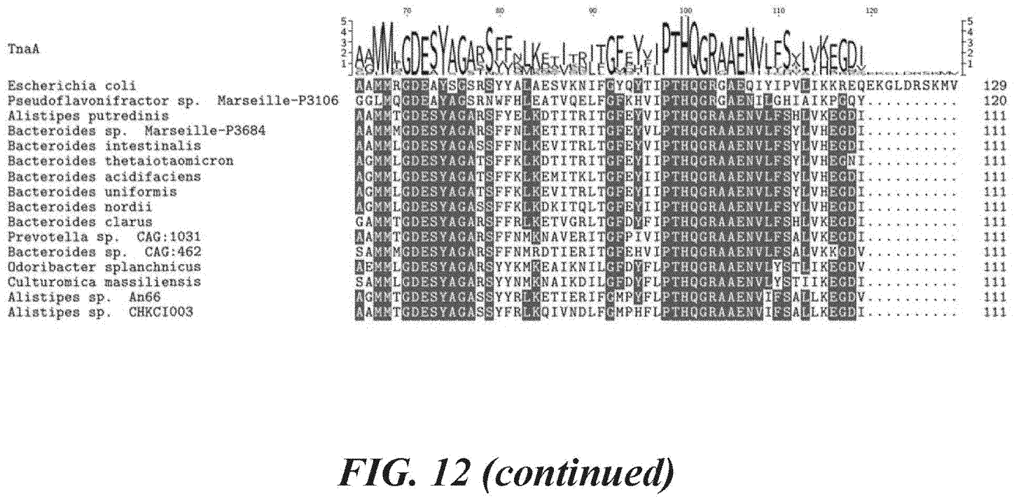

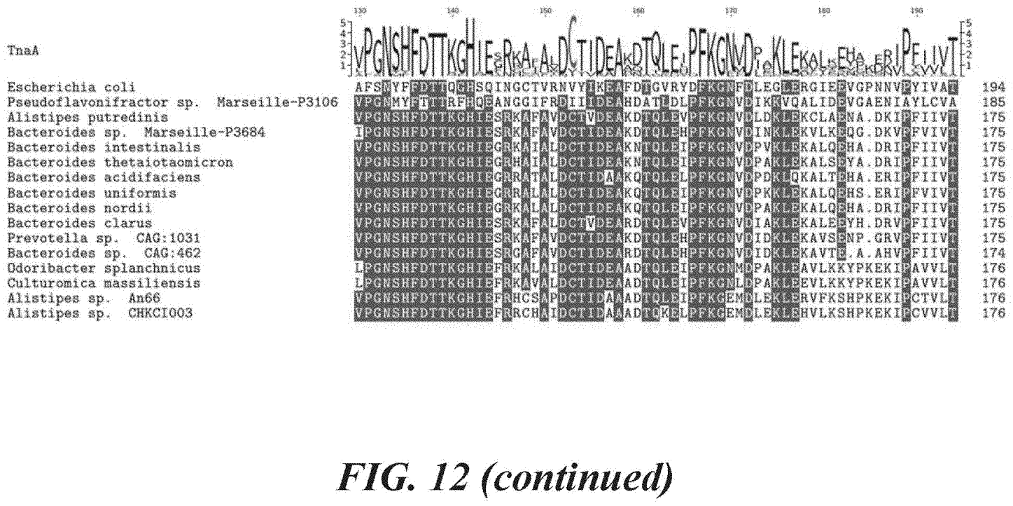

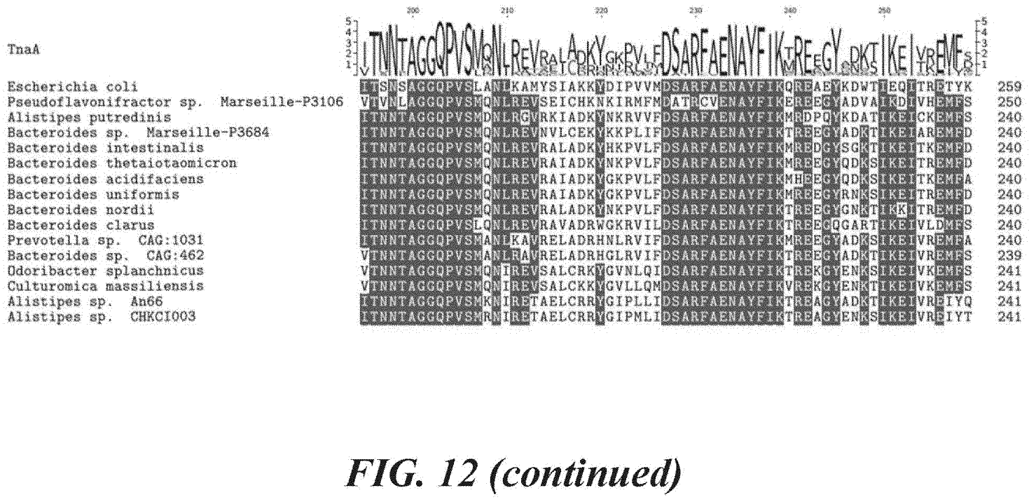

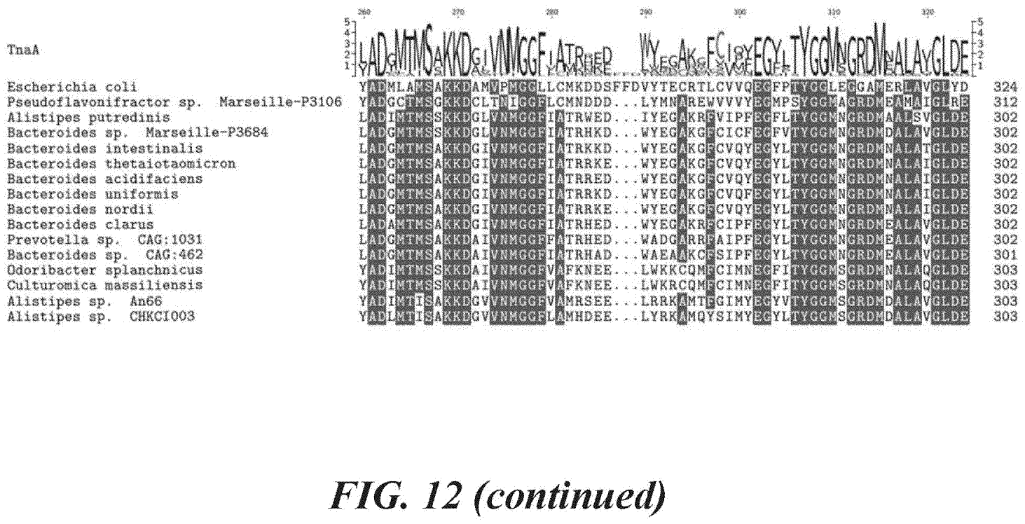

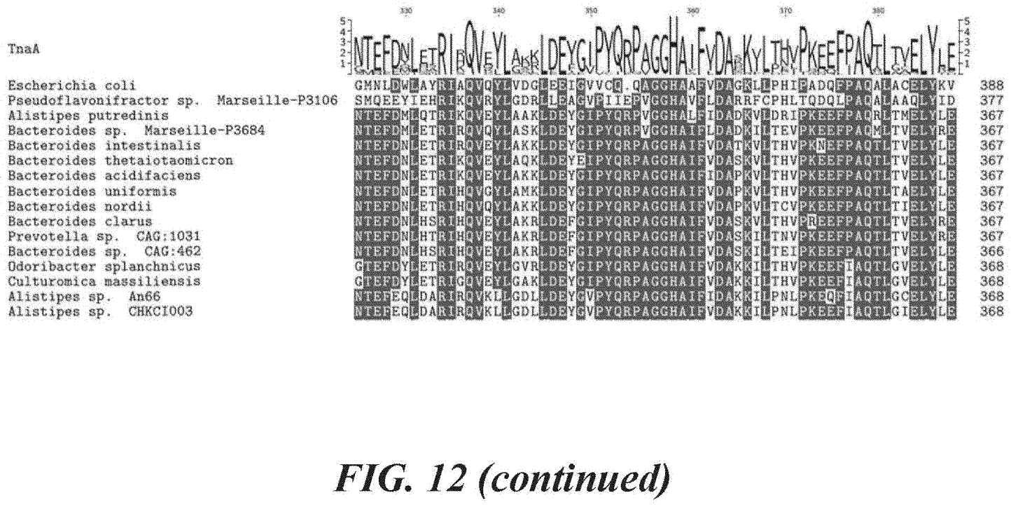

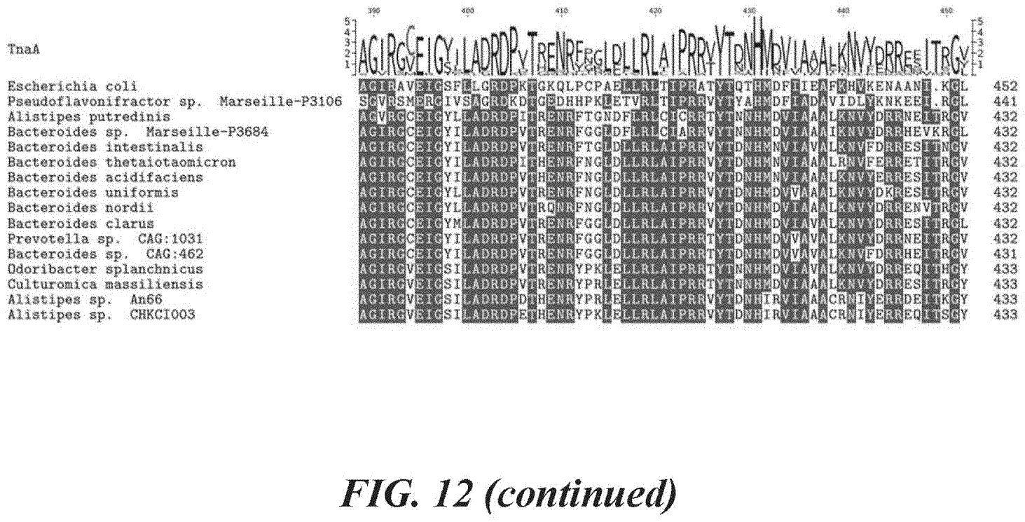

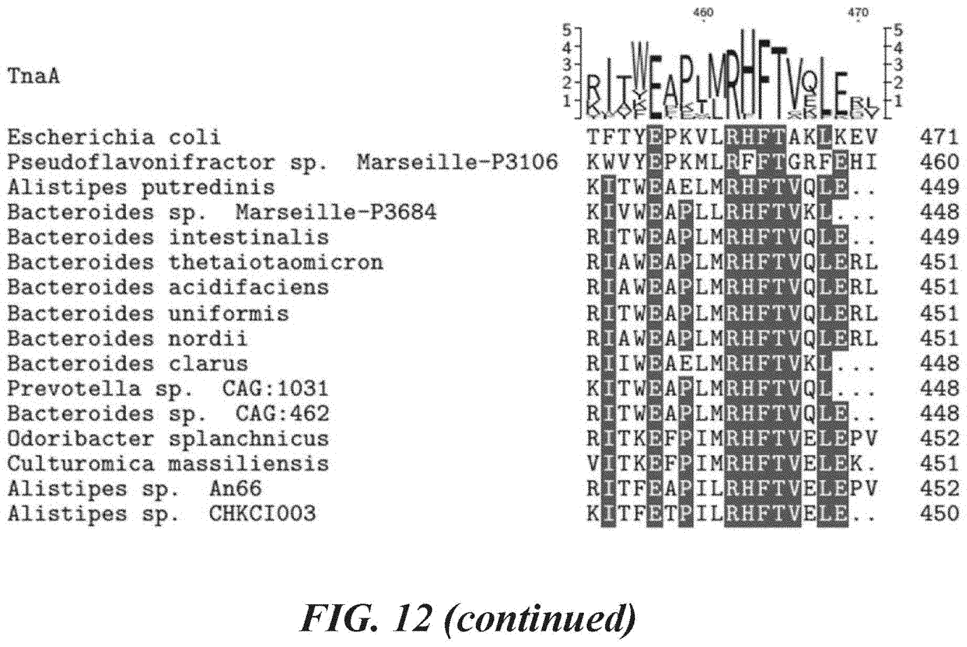

The working examples provided herein show that production of indoles by E. coli is differentially affected by levels of sulfide endogenously produced by gut bacteria. The bacterial metabolism can affect host physiology, and also microbe-microbe interactions driven by bacterial post-translational modifications mediated by host diet. Non-limiting examples of microorganisms that can affect renal disease pathology include those depicted in FIG. 12.

In another aspect, provided herein is a composition comprising:

a. an effective amount of a sulfated amino acid, e.g., an amount that increases the level of H₂S in the gastrointestinal tract of a subject; and

b. a carrier.

In some embodiments of any of the aspects, the composition comprises one or more of a sulfated amino acid selected from the group consisting of: methionine, cysteine, homocysteine, taurine, cystine (di-cysteine), salts, analogs, and derivatives thereof. In some embodiments of any of the aspects, the sulfated amino acid is isolated and purified from an external source. The composition can comprise a salt, derivative, or analog of a sulfated amino acid e.g., those described in U.S. Pat. Nos. 6,229,041 B1; 6,635,561 B1; 7,105,570 B2; 7,829,709 B1; and 10,144,717 B2 which are incorporated by reference in their entireties.

As used herein, a “sulfated amino acid” is any natural, isolated, purified, or synthetic amino acid comprising a sulfur atom (e.g., natural, isolated, purified, or synthetic amino acid comprising a thiol, sulfide, disulfide, sulfenic, sulfinic, sulfonic, sulfonate, sulfoxide, or sulfone group. Non-limiting examples of sulfated amino acids include cysteine (Cys) and methionine (Met). Additional non-limiting examples of sulfonated amino acids include homocysteine, taurine, cystine or di-cysteine, salts, analogs, and derivatives thereof.

The sulfated amino acids may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid.

These salts may be prepared by methods known to those skilled in the art, e.g., U.S. Pat. No. 7,105,570 B2 and WO 2001/027307 A1, which are incorporated herein by reference in their entireties.

In addition to salt forms, that the sulfated amino acids and compositions provided herein can be in a prodrug form. Prodrugs of the compositions described herein are those compositions that readily undergo chemical changes under physiological conditions to provide the compositions of the present invention. Additionally, prodrugs can be converted to the sulfated amino acids of the present invention by chemical or biochemical methods in an ex vivo environment. For example, prodrugs can be slowly converted to the sulfated amino acids when formulated in combination with a suitable enzyme or chemical reagent.

In some embodiments of any one of the aspects described herein, the agent that increases the level or activity of H₂S is a sulfur donor. For example, the sulfur donor can include the thiol group of a reactive Cys is modified to a persulfide (—SSH) group; disodium tetrasulfide (Na2S4), a poly-sulfide donor; sodium hydrosulfide, analogs, or derivatives thereof.

In some embodiments of any one of the aspects described herein, the agent or composition described herein modulates the level or activity of a bacterial cysteine desulfhydrase polypeptide. In some embodiments of any one of the aspects described herein, the agent or composition modulates the level of a bacterium expressing a cysteine desulfhydrase polypeptide. Bacteria that express a cysteine desulfhydrase polypeptide are known in the art, e.g., Nagasawa T, et al. “D-Cysteine desulfhydrase of Escherichia coli. Purification and characterization”. Eur Biochem. 1985 Dec. 16; 153(3):541-51; and Soutourina J, et al. “Role of D-cysteine desulfhydrase in the adaptation of Escherichia coli to D-cysteine.”J Biol Chem. 2001 Nov. 2; 276(44):40864-72, the contents of each of which are incorporated herein by reference in their entireties.

In some embodiments of any one of the aspects described herein, the agent or composition described herein is a bacterium that produces a sulfated amino acid described herein. Bacteria that can be used to produce sulfated amino acids or to be formulated in the compositions described herein include, but are not limited to those described, e.g., in U.S. Pat. Nos. 7,148,047 B2; and 8,383,372 B2.

In some embodiments of any one of the aspects described herein, the agent or composition modulates or increases the level or activity of a polypeptide or a nucleic acid encoding such a polypeptide selected from the group consisting of: cystathionine β-synthase; cystathionine γ-lyase, 3-mercaptopyruvate sulfurtransferase; and cysteine aminotransferase. These enzymes are involved in the transsulfuration pathway, a metabolic pathway involving the interconversion of cysteine and homocysteine through the intermediate cystathionine. The transulferation pathway is described in detail, e.g., in Aitken S M, Lodha P H, Morneau D J. The enzymes of the transsulfuration pathways: active-site characterizations. Biochim Biophys Acta. 2011 November; 1814(11):1511-7, the contents of each of which is incorporated herein by reference in its entirety.

The structure and function of cystathionine β-synthase and cystathionine γ-lyase polypeptides expressed in mammals and bacteria are known in the art. See, e.g., in Conter, C. et al. “Cystathionine β-synthase is involved in cysteine biosynthesis and H₂S generation in Toxoplasma gondii.” Sci Rep 10, 14657 (2020); Kozich V et al. “Cystathionine beta-synthase mutations: effect of mutation topology on folding and activity.” Hum. Mutat. 31:809-819 (2010); Nozaki T, et al., “Characterization of transsulfuration and cysteine biosynthetic pathways in the protozoan hemoflagellate, Trypanosoma cruzi. Isolation and molecular characterization of cystathionine beta-synthase and serine acetyltransferase from Trypanosoma.” J Biol Chem. 2001 Mar. 2; 276(9):6516-23. doi: 10.1074/jbc.M009774200. Epub 2000 Dec. 5. PMID: 11106665; Beatty P. et al. (1980). “Involvement of the cystathionine pathway in the biosynthesis of glutathione by isolated rat hepatocytes.” Arch. Biochem. Biophys. 204, 80-87; and Chiku T, et al. H₂S biogenesis by human cystathionine gamma-lyase leads to the novel sulfur metabolites lanthionine and homolanthionine and is responsive to the grade of hyperhomocysteinemia. J Biol Chem. 2009 Apr. 24; 284(17):11601-12, the contents of each of which are incorporated herein by reference in their entireties. The structure and function of 3-mercaptopyruvate sulfurtransferase is described, e.g., in Shibuya N, Mikami Y, Kimura Y, Nagahara N, Kimura H (November 2009). “Vascular endothelium expresses 3-mercaptopyruvate sulfurtransferase and produces hydrogen sulfide”. Journal of Biochemistry. 146 (5): 623-6 and Vachek H, Wood J L (January 1972). “Purification and properties of mercaptopyruvate sulfur transferase of Escherichia coli”. Biochimica et Biophysica Acta (BBA)—Enzymology. 258 (1): 133-46, the contents of each of which are incorporated herein by reference in their entireties. The structure and function of cysteine aminotransferase is described, e.g., in D'Aniello A. et al., “Amino acids and transaminases activity in ventricular CSF and in brain of normal and Alzheimer patients.” Neurosci Lett. 2005 Nov. 4; 388(1):49-53, the contents of each of which are incorporated herein by reference in their entireties.

In some embodiments of any of the aspects, the composition provided herein is formulated as a medical food. In some embodiments of any of the aspects, the composition provided herein is formulated as a pharmaceutical composition.

In some embodiments of any of the aspects, provided herein is a composition, pharmaceutical composition, medical food, or dietary supplement for use in the treatment of a renal disease and/or an inflammatory or fibrotic disease of the kidney.

In another aspect, provided herein is a pharmaceutical composition comprising: an agent that increases the level or activity of H₂S, e.g., in the gastrointestinal tract of a subject; and a carrier. The agent can be present in an amount sufficient for increasing the level or activity of H₂S in the gastrointestinal tract of a subject as compared to a reference level.

In another aspect, provided herein is a pharmaceutical composition comprising: an effective amount of a sulfated amino acid that increases the level or activity of H₂S in the gastrointestinal tract of a subject; and a carrier.

In some embodiments of any one of the aspects, provided herein is a composition comprising a pharmaceutically acceptable carrier or excipient.

In some embodiments of any of the aspects, the pharmaceutical composition is formulated to restrict delivery of the agent to the gastrointestinal tract of the subject. In some embodiments of any one of the aspects, the pharmaceutical composition comprises an enteric coating.

In some embodiments of any of the aspects, the composition further comprises a lipid vehicle. Exemplary lipid vehicles include, but are not limited to, liposomes, micelles, exosomes, lipid emulsions, and lipid-drug complex.

In some embodiments of any of the aspects, the pharmaceutical composition further comprises a particle or polymer-based vehicle. Exemplary particle or polymer-based vehicles include, but are not limited to, nanoparticles, microparticles, polymer microspheres, or polymer-drug conjugates.

In some embodiments of any one of the aspects, the pharmaceutical composition is a liquid dosage form or solid dosage form. Liquid dosage forms for oral administration include, but are not limited to, pharmaceutically acceptable emulsions, microemulsions, solutions, suspensions, syrups and elixirs.

The liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the agent described herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monosterate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragées, capsules, pills, can be used. Solid compositions of a similar type can also be employed as fillers in soft and hard-filled gelatin capsules using such excipients as lactose or milk sugar as well as high molecular weight polyethylene glycols, and the like. The solid dosage forms of tablets, dragées, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The solid dosage forms of tablets, dragées, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The compositions provided herein can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Pharmaceutical compositions include formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound or composition; as powders or granules; as solutions or suspensions in aqueous or non-aqueous liquids; or as oil-in-water or water-in-oil emulsions.

Accordingly, formulations suitable for rectal administration include gels, creams, lotions, aqueous or oily suspensions, dispersible powders or granules, emulsions, dissolvable solid materials, douches, and the like can be used. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

The methods provided herein comprise administering an effective amount of a composition comprising a sulfated amino acid to a subject in order to alleviate at least one symptom of the renal disease (e.g., an inflammatory and/or fibrotic disease of the kidney). As used herein, “alleviating at least one symptom of the renal disease” is ameliorating any condition or symptom associated with the renal disease (e.g., pain, abnormal urination, fever, vomiting, malaise, inflammation, fibrosis, etc.). As compared with an equivalent untreated control, such reduction is by at least 5%, 10%, 20%, 40%, 50%, 60%, 80%, 90%, 95%, 99% or more as measured by any standard technique. A variety of means for administering the compositions provided herein to subjects can be used.

In some embodiments of any of the aspects, the administering is oral administration, enteral administration, or parenteral administration. In some embodiments of any of the aspects, the agent is administered continuously, in intervals, or sporadically. The route of administration of the composition will be optimized for the type of composition being delivered (e.g., a pharmaceutical composition), and can be determined by a skilled practitioner.

The term “effective amount” as used herein refers to the amount of an agent (e.g., sulfated amino acid) or composition described herein can be administered to a subject. The subject may not have a renal disease. However, the effective amount of the sulfated amino acid provided herein increases the level of H₂S in the subject's gastrointestinal tract and/or serum. The subject may have or is diagnosed as having a renal disease and the effective amount of the composition or sulfated amino acid provided herein is needed to alleviate at least one or more symptom of the disease. The subject may be suspected of having a renal disease or reduce renal function compared to a healthy subject. The term “therapeutically effective amount” therefore refers to an amount of a composition that is sufficient to provide a particular anti-renal disease effect when administered to a typical subject. An effective amount as used herein, in various contexts, would also include an amount of an agent sufficient to delay the development of a symptom of the disease, alter the course of a symptom of the disease (e.g., slowing the progression of the renal disease), or reverse a symptom of the disease (e.g., correcting or halting symptoms of the renal disease). Thus, it is not generally practicable to specify an exact “effective amount.” However, for any given case, an appropriate “effective amount” can be determined by the assay provided herein and/or the subject's diet.

In some embodiments of any of the aspects, the composition is administered continuously (e.g., at constant levels over a period of time). Continuous administration of an agent can be achieved, e.g., by continuous release formulations or on-body injectors.

The effective dose can be estimated initially from cell culture assays. A dose can be formulated in animals. Generally, the compositions are administered so that a compound of the disclosure herein is used or given at a dose from 1 mg/kg to 1000 mg/kg; 1 mg/kg to 500 mg/kg; 1 mg/kg to 150 mg/kg, 1 mg/kg to 100 mg/kg, 1 mg/kg to 50 mg/kg, 1 mg/kg to 20 mg/kg, 1 mg/kg to 10 mg/kg, 1 μg/kg to 1 mg/kg, 100 mg/kg to 100 mg/kg, 100 mg/kg to 50 mg/kg, 100 mg/kg to 20 mg/kg, 100 mg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

In some embodiments of any of the aspects, the administering comprises orally administering a composition comprising at least about 0.8 grams or more, 0.9 grams or more, 1 gram or more, 2 grams or more, 3 grams or more 4 grams or more, 5 grams of more, 6 grams or more of cysteine or a derivative thereof per day. In some embodiments of any of the aspects, the administering comprises orally administering a composition comprising at least about 0.8 grams or more, 0.9 grams or more, 1 gram or more, 2 grams or more, 3 grams or more 4 grams or more, 5 grams of more, 6 grams or more of methionine or a derivative thereof per day.

The compositions described herein can be administered at once, or can be divided into a number of smaller doses to be administered at intervals of time. It is understood that the precise dosage and duration of treatment will be a function of the location of where the composition is administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

In some embodiments of any of the aspects, the composition provided herein is administered in intervals (e.g., at various levels over a given period of time). In some embodiments of any one of the aspects, the composition provided herein is administered hourly, daily, weekly, or monthly. By way of example only, the administering is every 4 hours, every 6 hours, every 12 hours, daily, weekly, or monthly. In some embodiments of any one of the aspects, the administering is daily for a period of 1 week or more, 2 weeks or more, 3 weeks or more, 4 weeks or more, 5 weeks or more, 6 weeks or more, 7 weeks or more, 8 weeks or more, 9 weeks or more, or 10 weeks or more. In some embodiments of any one of the aspects, the administering is twice daily for a period of 1 week or more, 2 weeks or more, 3 weeks or more, 4 weeks or more, 5 weeks or more, 6 weeks or more, 7 weeks or more, 8 weeks or more, 9 weeks or more, or 10 weeks or more. In some embodiments of any one of the aspects, the administering is three times daily for a period of 1 week or more, 2 weeks or more, 3 weeks or more, 4 weeks or more, 5 weeks or more, 6 weeks or more, 7 weeks or more, 8 weeks or more, 9 weeks or more, or 10 weeks or more.

In some embodiments of any of the aspects, the composition provided herein is re-administered to the subject to regulate the level or activity of hydrogen sulfide (H₂S) in the gastrointestinal tract of the subject.

In some embodiments of any of the aspects, the administering increases the level of H₂S in a subject (e.g., the gastrointestinal tract or serum) by at least 5%, 10%, 20%, 40%, 50%, 60%, 80%, 90%, 95%, 99% or more compared with a reference level or an appropriate control as measured by any standard technique.

Effective amounts, toxicity, and therapeutic efficacy can be evaluated by standard pharmaceutical procedures in cell cultures or experimental animals. The dosage can vary depending upon the dosage form employed and the route of administration utilized. The effects of any particular dosage can be monitored by a suitable bioassay, e.g., measuring renal function, urinalysis, or blood work, among others. The dosage can be determined by a physician and adjusted, as necessary, to suit observed effects of the treatment.

The dosage of the composition provided herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further agents, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication. In some embodiments of any one of the aspects, the dosage and administration of the composition provided herein is determined by the measuring the levels of H₂S in the gastrointestinal tract of the subject.

In some embodiments of any of the aspects, the agent or composition described herein is used as a monotherapy. In some embodiments of any of the aspects, the agents described herein can be used in combination with other known agents and therapies for a renal disease. Administered “in combination,” as used herein, means that two (or more) different treatments are delivered to the subject during the course of the subject's affliction with the disorder, e.g., the two or more treatments are delivered after the subject has been diagnosed with the disorder (e.g., a renal disease) and before the disorder has been cured or eliminated or treatment has ceased for other reasons. In some embodiments of any one of the aspects, the delivery of one treatment is still occurring when the delivery of the second begins, so that there is overlap in terms of administration. This is sometimes referred to herein as “simultaneous” or “concurrent delivery.” In other embodiments of any one of the aspects, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments of any one of the aspects, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Therapeutics, dietary supplements, and treatments currently used to treat a renal disease include, but are not limited to, antibiotics (e.g. aminosalicylic acid, norfloxacin, penicillin, cephalosporin), analgesics (e.g. acetaminophen, ibuprofen), non-steroidal anti-inflammatory drugs, anti-inflammatory drugs, vitamins (e.g., vitamin D), erythropoietin, omega 3-fatty acids, calcium carbonate, potassium, IV fluids, angiotensin-converting enzyme inhibitors (ACEis) or angiotensin-receptor blockers (ARBs), dialysis, other treatments for renal disease known in the art.

When administered in combination, the composition provided herein and the additional agent (e.g., second or third agent), or all, can be administered in an amount or dose that is higher, lower or the same as the amount or dosage of each agent used individually, e.g., as a monotherapy. In certain embodiments of any one of the aspects, the administered amount or dosage of the agent, the additional agent (e.g., second or third agent), or all, is lower (e.g., at least 20%, at least 30%, at least 40%, or at least 50%) than the amount or dosage of each agent used individually. In other embodiments of any one of the aspects, the amount or dosage of agent, the additional agent (e.g., second or third agent), or all, that results in a desired effect (e.g., treatment of a renal disease) is lower (e.g., at least 20%, at least 30%, at least 40%, or at least 50% lower) than the amount or dosage of each agent individually required to achieve the same therapeutic effect.

Increasing the levels of one or more sulfated amino acids provided herein can be beneficial and/or therapeutic to the subject. In some embodiments of any of the aspects, the composition provided herein is formulated as a dietary supplement.

In some embodiments of any of the aspects, the dietary supplement and/or composition provided herein comprises a level of a sulfated amino acid which is 2% or more, 3% or more, 5% or more, 10% or more, 15% or more, 20% or more, 35% or more, 30% or more compared to a prior diet or a reference level. The prior diet can be a diet of the subject's choosing, the diet before administration of the dietary supplement or composition provided herein, or a prior diet provided to the subject.

In some embodiments of any of the aspects, the dietary supplement or composition provided herein comprises one or more of the components and/or food ingredients in the following table:

TABLE 1
DIET FORMULATIONS

| | Product # | | | |
|---|---|---|---|---|
| | A15121501 | | A15121502 | |
| | High SAA | | Low SAA | |
| | gm % | kcal % | gm % | kcal % |
| Protein | 17 | 18 | 17 | 18 |
| Carbohydrate | 69 | 71 | 69 | 71 |
| Fat | 5 | 12 | 5 | 12 |
| Total | | 100 | | 100 |
| kcal/gm | | 3.9 | | 3.9 |
| Ingredient (gm) | gm | kcal | gm | kcal |
| L-Arginine | 9.2 | 37 | 10.4 | 41.6 |
| L-Histidine-HCl-H2O | 5.5 | 22 | 6.3 | 25.2 |
| L-Isoleucine | 7.4 | 30 | 8.3 | 33.2 |
| L-Leucine | 11 | 44 | 12.5 | 50 |
| L-Lysine-HCl | 12.9 | 52 | 14.6 | 58.4 |
| L-Methionine | 15 | 60 | 3 | 12 |
| L-Phenylalanine | 7.4 | 30 | 8.3 | 33.2 |
| L-Threonine | 7.4 | 30 | 8.3 | 33.2 |
| L-Tryptophan | 1.8 | 7 | 2.1 | 8.4 |
| L-Valine | 7.4 | 30 | 8.3 | 33.2 |
| L-Alanine | 8.9 | 36 | 10.5 | 42 |
| L-Asparagine-H2O | 4.6 | 18 | 5.2 | 20.8 |
| L-Aspartate | 9.2 | 37 | 10.4 | 41.6 |
| L-Cystine | 8 | 32 | 0.3 | 1.2 |
| L-Glutamic Acid | 27.6 | 110 | 31.3 | 125.2 |
| L-Glutamine | 4.6 | 18 | 5.2 | 20.8 |
| Glycine | 9.2 | 37 | 10.4 | 41.6 |
| L-Proline | 4.6 | 18 | 5.2 | 20.8 |
| L-Serine | 4.6 | 18 | 5.2 | 20.8 |
| L-Tyrosine | 3.7 | 15 | 4.2 | 16.8 |
| L-Amino Acids, total | 170 | 680 | 170 | 680 |
| Corn Starch | 550.5 | 2202 | 550.5 | 2202 |
| Maltodextrin 10 | 125 | 500 | 125 | 500 |
| Sucrose | 0 | 0 | 0 | 0 |
| Cellulose | 50 | 0 | 50 | 0 |
| Corn Oil | 50 | 450 | 50 | 450 |
| Mineral Mix S10001 | 35 | 0 | 35 | 0 |
| Sodium Bicarbonate | 7.5 | 0 | 7.5 | 0 |
| Vitamin Mix V10001 | 10 | 40 | 10 | 40 |
| Choline Bitartrate | 2 | 0 | 2 | 0 |
| FD&C Yellow Dye #5 | 0.05 | 0 | 0 | 0 |
| FD&C Red Dye #40 | 0 | 0 | 0.05 | 0 |
| FD&C Blue Dye #1 | 0 | 0 | 0 | 0 |
| Total | 1000.05 | 3872 | 1000.05 | 3872 |

In some embodiments of any one of the aspects, the composition provided herein further comprises adenine. For example, Saa-Adenine diets can comprise 0.01% or more, 0.02% or more, 0.03% or more, 0.04% or more, 0.05% or more adenine.

The dietary supplement or composition as provided herein can be provided or administered for a set period of time, until one or more symptoms and/or markers of a condition is alleviated, (e.g., a renal disease), and/or until a marker of dietary success is detected (e.g., H₂S). As used herein, “alleviating a symptom” is ameliorating any condition or symptom associated with the condition. As compared with an equivalent untreated control, such amelioration is by at least 5%, 10%, 20%, 40%, 50%, 60%, 80%, 90%, 95%, 99% or more as measured by any standard technique. Markers of dietary success are markers of increase H₂S, reduced inflammation, reduced fibrosis, and/or alleviating at least one symptom of a renal disease and/or an inflammatory or fibrotic disease of the kidney.

In some embodiments of any of the aspects, the composition comprises at least one food ingredient. In some embodiments of any of the aspects, the carrier is a food ingredient.

As used herein, “food ingredient” refers to any product, composition, or a component of a food known to have or disclosed as having a nutritional effect. Food can include various meats (e.g., beef, pork, poultry, fish, etc.), dairy products (e.g., milk, cheese, eggs), fruits, vegetables, cereals, breads, etc., and components thereof. Food can be fresh or preserved, e.g., by canning, dehydration, freezing, or smoking. Food can be provided in raw, unprepared and/or natural states or in cooked, prepared, and/or combined states.

In some embodiments of any of the aspects, the food ingredient is selected from the group consisting of: fat, carbohydrates, protein, fiber, nutritional balancing agent, and mixtures thereof. In some embodiments of any of the aspects, the composition provided herein further comprises one or more of a protein or an amino acid. In some embodiments of any of the aspects, the composition further comprises adenine, one or more vitamins, potassium, omega 3-fatty acids, and/or calcium carbonate.

In some embodiments of any of the aspects, the composition is a pet food. In some embodiments of any one of the aspects, the pet food is a cat food or a dog food formulated to enhance or improve renal function. Methods of producing pet foods are described, e.g., in U.S. Pat. Nos. 10,238,136 B2 and 10,849,338 B2, the contents of each of which are incorporated herein by reference in their entireties.

In some embodiments of any one of the aspects, the composition is a protein powder comprising one or more sulfated amino acids selected from the group consisting of: methionine, cysteine, homocysteine, taurine, cystine (di-cysteine), salts, analogs, and derivatives thereof.

In some embodiments of any one of the aspects, the composition provided herein can be a shake, meal replacement shake, drink, smoothie, powder, bars, or the like.

The composition provided herein can be provided in meals and/or portions, e.g., a grouping or unit of food ingredients which are intended to be consumed as a meal.

In another aspect, provided herein is a method of treating an inflammatory or fibrotic disease of the kidney in a subject, the method comprises: administering to a subject in need thereof a composition comprising a sulfated amino acid.

In another aspect, provided herein is a method of treating an inflammatory or fibrotic disease of the kidney in a subject, the method comprising: orally administering to a subject in need thereof a composition comprising a sulfated amino acid.

In some embodiments of any of the aspects, the methods provided herein comprise administering a dietary supplement or a food composition described herein to a subject having or diagnosed as having a renal disease e.g., chronic kidney disease, renal parenchymal injury, tubulitis, end-stage renal failure, lupus, nephritis, acute renal failure, kidney infection, polycystic kidney disease, renal amyloidosis, and/or renal colic. Subjects having one of these conditions can be identified by a physician using current methods of diagnosing such conditions. Symptoms and/or complications of these conditions which characterize these conditions and aid in diagnosis are well known in the art.

In some embodiments of any of the aspects, the subject provided herein has or is suspected of having an enrichment of one or more bacteria selected from the group consisting of: Enterobacteriaceae, Escherichia, Escherichia coli, Bacterioides, Prevotella, Ordoribacter, Cuhuromica, Alistipes, Pseudoflavonifractor, Pseudoflavonifractor sp. Marseille-P3106, Alistipes putredinis, Bacteroides intestinalis, Bacteroides thetaiotaomicron, Bacteroides acidifaciens, Bacteroides uniformis, Bacteroides nordii, Bacteroides clarus, Prevotella sp. CAG 1031, Bacteroides sp. CAG 462, Ordoribacter splanchnicus, Culturomica massiliensis, Alistipes sp. An66, and Alistipes sp. CHKCI003 in the gastrointestinal tract.

In another aspect, provided herein is an assay for identifying an agent for the treatment of an inflammatory or fibrotic disease of the kidney in a subject, the assay comprising:

a. contacting a bacterium with an agent; and

b. detecting the level or activity of hydrogen sulfide (H₂S).

Methods of detecting the levels of H₂S include but are not limited to a lead acetate assay, a methylene blue assay, chromatography, gas chromatography, chemiluminescence-based assays, mass-spectrometry, and any other method known in the art. Methods of determining H₂S activity include but are not limited to measuring serum creatinine levels, histology from a biological sample or biopsy, measuring the level of sulfated proteins, measuring the level of S-sulfhydrated TnaA, and measuring indole concentration. The level or activity of H₂S measured or detected by the methods provided herein can be compared to a reference level or an appropriate control.

In some embodiments of any of the aspects, prior to step (a) of contacting the bacterium with an agent, a biological sample is obtained from a subject with a renal disease. In some embodiments of any one of the aspects, the biological sample is a cecal sample, urine sample, blood sample, or a serum sample.

In another aspect, provided herein is a method of treating a renal disease in a subject, the method comprising: (a) measuring the level of H₂S in a biological sample obtained from a subject; and (b) comparing the measurement of (a) to a reference level; (c) identifying a subject with decreased H₂S in (a) as compared to a reference level as having a renal disease; and (d) administering to the subject having a renal disease an agent that modulates H₂S.

In another aspect, provided herein is a method of treating a renal disease in a subject, the method comprising: (a) receiving the results of an assay that indicates that the subject has a decrease in H₂S in the gastrointestinal tract; and (b) administering to the subject an agent or composition described herein that modulates the level or activity of H₂S.

### Some Selected Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments of any one of the aspects, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology, cellular and molecular biology, and biochemistry can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with renal disease, e.g. chronic kidney disease. The term “treating” includes reducing or alleviating at least one adverse effect or symptom of renal disease, for example, increased or decreased urination, pain, loss of appetite, discomfort, or vomiting. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the terms “regulates” or “modulates” are used interchangeably to refer to an effect including increasing or decreasing a given parameter as those terms are defined herein. For example, in some embodiments the method comprising regulating hydrogen sulfide in the gastrointestinal tract.

As used herein “preventing” or “prevention” refers to any methodology where the disease state does not occur due to the actions of the methodology (such as, for example, administration of an agent as described herein). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

As used herein the terms “renal disease” or “kidney disease” are used interchangeable to refer to any disease that affects the kidney or kidney function. The renal disease can cause at least one symptom of a disease. These symptoms can include but are not limited to, frequent or lack of urination, extreme thirst, pain, malaise, fever, or any other symptom associated with a renal disease in a subject.

Also used herein, the term “an inflammatory or fibrotic disease of the kidney” can refer to kidney diseases that have such inflammatory and fibrotic pathology. Non-limiting examples of renal diseases and inflammatory and fibrotic diseases of the kidney include chronic kidney disease, renal parenchymal injury, tubulitis, end-stage renal failure, lupus, nephritis, acute renal failure, kidney infection, polycystic kidney disease, renal amyloidosis, and renal colic.

As used herein, the terms “administering,” is used in the context of the placement of an agent (e.g. a sulfated amino acid) described herein, into a subject, by a method or route which results in at least partial localization of the agent at a desired site, such as the gastrointestinal tract, kidney, or a region thereof, such that a desired effect(s) is produced (e.g., increase H₂S level or activity). The agent described herein can be administered by any appropriate route which results in delivery to a desired location in the subject. The half-life of the agent after administration to a subject can be as short as a few minutes, hours, or days, e.g., twenty-four hours, to a few days, to as long as several years, i.e., long-term. In some embodiments of any of the aspects, the term “administering” refers to the administration of a pharmaceutical composition comprising one or more agents. The administering can be done by oral administration, enteric administration (J tube), parenteral administration, direct injection (e.g., directly administered to a target cell or tissue), subcutaneous injection, muscular injection, to the subject in need thereof. Administering can be local or systemic.

The terms “patient”, “subject” and “individual” are used interchangeably herein, and refer to an animal, particularly a human, dog, or cat, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In some embodiments of any of the aspects, the subject has or is suspected of having an inflammatory or fibrotic disease of the kidney. In some embodiments of any of the aspects provided herein, the subject is a mammal. In some embodiments of any of the aspects, the subject is human. In some embodiments of any one of the aspects, of any of the aspects, the subject is a domesticated animal including companion animals (e.g., dogs, cats, rats, guinea pigs, hamsters etc.). In some embodiments of any of the aspects, the subject is a dog, or a cat. In some embodiments of any one of the aspects, of any of the aspects, the subject is an experimental animal or animal substitute as a disease model. A subject can have previously received a treatment for a renal disease, or has never received treatment for a renal disease. A subject can have previously been diagnosed with having a renal disease, or has never been diagnosed with a renal disease.

A “subject in need” of treatment for a particular condition can be a subject having that condition, diagnosed as having that condition, or at risk of developing that condition (e.g., a renal disease).

As used herein, the term “inflammation” or “inflamed” refers to activation or recruitment of the immune system or immune cells (e.g. T cells, B cells, macrophages, etc.). A tissue that has inflammation can become reddened, white, swollen, hot, painful, exhibit a loss of function, or have a film or mucus. Methods of identifying inflammation are well known in the art. Inflammation typically occurs following injury or infection by a microorganism. In some embodiments of any one of the aspects, the inflammation is kidney inflammation.

As used herein, the term “pharmaceutical composition” refers to an active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an artificial or engineered carrier, e.g., a carrier in which the active ingredient would not be found to occur in nature.

As used herein, the term “salt” refers to acid or base salts of the sulfated amino acids or compositions used in the methods of the present invention. Illustrative examples of salts include mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. The term salt also refers to formation of a salt between two compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The term “agent” as used herein means any compound or substance such as, but not limited to, a small molecule, nucleic acid, polypeptide, peptide, drug, ion, a sulfate amino acid, etc. An “agent” can be any chemical, entity or moiety, including without limitation, synthetic and naturally-occurring proteinaceous and non-proteinaceous entities. In some embodiments of any of the aspects, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments of any one of the aspects, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a organic or inorganic molecule, either natural (i.e., found in nature) or non-natural (i.e., not found in nature), which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Examples of “small molecules” that occur in nature include, but are not limited to, taxol, dynemicin, and rapamycin. Examples of “small molecules” that are synthesized in the laboratory include, but are not limited to, compounds described in Tan et al., (“Stereoselective Synthesis of over Two Million Compounds Having Structural Features Both Reminiscent of Natural Products and Compatible with Miniaturized Cell-Based Assays” J. Am. Chem. Soc. 120:8565, 1998; incorporated herein by reference). In certain other preferred embodiments of any one of the aspects, natural-product-like small molecules are utilized.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. In some embodiments, the term “pharmaceutically acceptable carrier” excludes tissue culture media. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Non-limiting examples of pharmaceutical carriers include particle or polymer-based vehicles such as nanoparticles, microparticles, polymer microspheres, or polymer-drug conjugates.

As used herein, the term “restricts delivery of the composition to the gastrointestinal tract” refers to a formulation that permits or facilitates the delivery of the agent or pharmaceutical composition described herein to the colon, large intestine, or small intestine in viable form. Enteric coating or micro- or nano-particle formulations can facilitate such delivery as can, for example, buffer or other protective formulations.

As used herein, the terms “heteroatom” or “ring heteroatom” are meant to include, oxygen (O), nitrogen (N), sulfur (S), phosphorus (P), Boron (B), Arsenic (As), and silicon (Si).

The term “thiol” or “sulfhydryl”, alone or in combination, means a —SH group (e.g., R—SH). The term “thio” or “thia”, alone or in combination, means a thioether group; i.e., an ether group wherein the ether oxygen is replaced by a sulfur atom.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized.

The term “heteroaryl” refers to aryl groups (or rings) that contain at least one heteroatom such as N, O, or S, wherein the nitrogen and sulfur atoms are optionally oxidized, and the nitrogen atom(s) are optionally quaternized.

The term “derivative” as used herein means any chemical, conservative substitution, or structural modification of an agent (e.g., a sulfated amino acid). The derivative can improve characteristics of the agent or small molecule such as pharmacodynamics, pharmacokinetics, absorption, distribution, delivery, targeting to a specific receptor, or efficacy. For example, for a small molecule, the derivative can consist essentially of at least one chemical modification to about ten modifications. The derivative can also be the corresponding salt of the agent (e.g sodium salts). The derivative can be the pro-drug of the small molecule as described herein.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

In some embodiments of any of the aspects, a polypeptide or sulfated amino acid as described herein can be engineered. As used herein, “engineered” refers to the aspect of having been manipulated by the hand of man. For example, a peptide is considered to be “engineered” when at least one aspect of the peptide, e.g., its sequence, has been manipulated by the hand of man to differ from the aspect as it exists in nature.

The terms “decrease”, “reduced”, “reduction”, or “inhibit” are all used herein to mean a decrease or lessening of a property, level, or other parameter by a statistically significant amount. In some embodiments of any one of the aspects, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. For example, increasing the level of H₂S or activity of H₂S in the gastrointestinal tract of a subject.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a renal disease, or a biological sample that has not been contacted with an agent or composition disclosed herein).

As used herein, an “appropriate control” refers to an untreated, otherwise identical cell or population (e.g., a biological sample that was not contacted by an agent or composition described herein, or not contacted in the same manner, e.g., for a different duration, as compared to a non-control cell). In some embodiments of any of the aspects, an appropriate control would be the levels or activity of H₂S in an otherwise identical sample that is not contacted by an agent or composition described herein, or is the level of H₂S activity in a subject prior to administration of an agent or composition. Further, an appropriate control can be the level of H₂S activity in a healthy subject, e.g., an individual that does not have a disease. One skilled in the art can determine the activity of H₂S using functional readouts of H₂S activity, for example, by measuring/assessing S-sulfhydration of a peptide (e.g., TnaA) or the production of indole and/or indoxyl sulfate. One skilled in the art can assess/measure the levels of H₂S and downstream targets of interest, e.g., using biochemical assays, respectively.

The term “pharmaceutically acceptable” can refer to compounds and compositions which can be administered to a subject (e.g., a mammal or a human) without undue toxicity.

As used herein, “detecting” is understood to mean that an assay was performed for a specific target or protein (e.g. H₂S). The amount of target detected can be none or below the level of detection of the assay. Examples of assays include but are not limited to, a lead acetate assay, pull-down assays, mass spectrometry, liquid chromatography, western blotting, colorimetric assays, ELISA assays, tryptophanase assays, RT-PCR, nucleic acid sequencing, and histology.

As used herein, the term “regulates” or “modulates” refers to an effect including increasing or decreasing a given parameter as those terms are defined herein.

As used herein, the term “contacting” when used in reference to a cell or organ, encompasses both introducing or administering an agent, sulfated amino acid, surface, hormone, etc. to the cell, tissue, or organ in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventor(s) are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

Some embodiments of the technology described herein can be defined according to any of the following numbered paragraphs:
1) A method of regulating the level or activity of hydrogen sulfide (H₂S) in the gastrointestinal tract of a subject, the method comprising: administering to the subject a composition comprising a sulfated amino acid.
2) The method of paragraph 1, wherein the composition comprises one or more of a sulfated amino acid selected from the group consisting of: methionine, cysteine, homocysteine, taurine, cystine (di-cysteine), salts, analogs, and derivatives thereof.
3) The method of any one of paragraphs 1-2, wherein the composition comprises at least one food ingredient.
4) The method of paragraph 3, wherein the food ingredient is selected from the group consisting of: fats, carbohydrates, proteins, fibers, nutritional balancing agents, and mixtures thereof.
5) The method of any one of paragraphs 1-4, wherein the composition is formulated as a dietary supplement.
6) The method of any one of paragraphs 1-5, wherein the composition is formulated as a medical food.
7) The method of any one of paragraphs 1-6, wherein the composition is formulated as a pharmaceutical composition.
8) The method of any one of paragraphs 1-7, wherein the administering is oral administration, enteral administration, or parenteral administration.
9) The method of any one of paragraphs 1-8, wherein the subject is a mammal.
10) The method of any one of paragraphs 1-9, wherein the subject is a human, a dog, or a cat.
11) The method of any one of paragraphs 1-10, wherein the subject has or is suspected of having an inflammatory or fibrotic disease of the kidney.
12) A method of treating an inflammatory or fibrotic disease of the kidney in a subject, the method comprising: administering to a subject in need thereof a composition comprising a sulfated amino acid.
13) The method of paragraph 12, wherein the composition comprises one or more of a sulfated amino acid selected from the group consisting of: methionine, cysteine, homocysteine, taurine, cystine (di-cysteine), salts, analogs, and derivatives thereof.
14) The method of any one of paragraphs 12-13, wherein the composition is formulated as a dietary supplement.
15) The method of any one of paragraphs 12-14, wherein the composition is formulated as a medical food.
16) The method of any one of paragraphs 12-14, wherein the composition is formulated as a pharmaceutical composition.
17) The method of any one of paragraphs 12-16, wherein the administering is oral administration, enteral administration, or parenteral administration.
18) The method of any one of paragraphs 12-17, wherein the subject is a mammal.
19) The method of any one of paragraphs 12-18, wherein the subject is a human, a dog, or a cat.
20) The method of paragraph 11 or 12, wherein the inflammatory or fibrotic disease of the kidney is selected from the group consisting of: chronic kidney disease, renal parenchymal injury, tubulitis, end-stage renal failure, lupus, nephritis, acute renal failure, kidney infection, polycystic kidney disease, renal amyloidosis, and renal colic.
21) The method of paragraph 1 or 12, wherein the subject has or is suspected of having an enrichment of one or more bacteria selected from the group consisting of: Enterobacteriaceae, Escherichia, Escherichia coli, Bacterioides, Prevotella, Ordoribacter, Cuhuromica, Alistipes, Pseudoflavonifractor, Pseudoflavonifractor sp. Marseille-P3106, Alistipes putredinis, Bacteroides intestinalis, Bacteroides thetaiotaomicron, Bacteroides acidifaciens, Bacteroides uniformis, Bacteroides nordii, Bacteroides clarus, Prevotella sp. CAG 1031, Bacteroides sp. CAG 462, Ordoribacter splanchnicus, Culturomica massihensis, Alistipes sp. An66, and Alistipes sp. CHKCI003 in the gastrointestinal tract.
22) A method of treating an inflammatory or fibrotic disease of the kidney in a subject, the method comprising: orally administering to a subject in need thereof a composition comprising a sulfated amino acid.
23) An assay for identifying an agent for the treatment of an inflammatory or fibrotic disease of the kidney in a subject, the assay comprising:
   a. contacting a bacterium with an agent; and
   b. detecting the level or activity of hydrogen sulfide (H₂S).
24) The assay of paragraph 23, wherein the bacterium is selected from the group consisting of: Enterobacteriaceae, Escherichia, Escherichia coli, Bacterioides, Prevotella, Ordoribacter, Cuhuromica, Alistipes, Pseudoflavonifractor, Pseudoflavonifractor sp. Marseille-P3106, Alistipes putredinis, Bacteroides intestinalis, Bacteroides thetaiotaomicron, Bacteroides acidifaciens, Bacteroides uniformis, Bacteroides nordii, Bacteroides clarus, Prevotella sp. CAG 1031, Bacteroides sp. CAG 462, Ordoribacter splanchnicus, Culturomica massihensis, Alistipes sp. An66, and Alistipes sp. CHKCI003.
25) The assay of any one of paragraphs 23-24, wherein the assay further comprises detecting the level of S-sulfhydrated polypeptides.
26) The assay of any one of paragraphs 23-25, wherein the assay further comprises detecting the level or activity of decR, yhaOM, tryptophanase (TnaA), indole, and/or indoxyl sulfate.
27) A composition comprising:
   a. an effective amount of a sulfated amino acid that increases the level or activity of H₂S in the gastrointestinal tract of a subject; and
   b. a carrier.
28) The composition of paragraph 27, wherein the carrier is a food ingredient.
29) The composition of any one of paragraphs 27-28, wherein the sulfated amino acid is selected from the group consisting of: methionine, cysteine, homocysteine, taurine, cystine (di-cysteine), salts, analogs, and derivatives thereof.
30) The composition of paragraph 28, wherein the food ingredient is selected from the group consisting of: fats, carbohydrates, proteins, fibers, nutritional balancing agents, and mixtures thereof.
31) The composition of any one of paragraphs 27-30, further comprising adenine, one or more vitamins, potassium, omega 3-fatty acids, and/or calcium carbonate.

### EXAMPLES

### Example 1: Dietary Cysteine Affects Renal Function by S-Sulfhydration of Bacterial Tryptophanase

### Highlights:

(1) Dietary cysteine and the microbiota modulate kidney injury in mice

(2) Enterobacteriaceae (E. coli) are enriched in the gut microbiomes of CKD patients

(3) E. coli tryptophanase is S-sulfhydrated by cysteine, inhibiting its function

(4) TnaA S-sulfhydration status in vivo correlates with kidney function

Associations between chronic kidney disease (CKD) and the gut microbiota have been postulated, yet questions remain about their reproducibility and underlying mechanisms. Since dietary sulfur and protein may modulate CKD severity, the role of dietary sulfur amino acids (Saa) were evaluated, which also affect gut sulfide levels, in a mouse CKD model. Further, data was analyzed that revealed an enrichment of Enterobacteriaceae (e.g. Escherichia coli) in CKD patients. It was discovered that Saa alter S-sulfhydration of the E. coli enzyme tryptophanase (TnaA), which converts tryptophan to indole. TnaA S-sulfhydration levels in vivo correlated with amounts of the uremic toxin indoxyl sulfate and kidney function, demonstrating a relationship between diet, post-translational modification and activity of microbial enzymes. Collectively, these findings reveal the basis for an interaction between dietary components, microbial metabolism and kidney function and provide a framework for understanding how dietary modification and/or inhibiting TnaA activity might alleviate CKD progression.

Chronic kidney disease (CKD) affects nearly 850 million people worldwide (Crews et al., 2019). Diet can alter gut microbiota composition and activity (David et al., 2014; Wu et al., 2016) and, while dietary modification is a cornerstone of CKD treatment, the role of the microbiota in leveraging this effect has not been well-characterized. Some CKD patients harbor a distinct gut microbiota compared to non-CKD control subjects (Castillo-Rodriguez et al., 2018), and gut bacteria can function in production of uremic toxins such as indoxyl-sulfate (Devlin et al., 2016) and p-cresol that contribute to CKD morbidity and mortality (Y.-Y. Chen et al., 2019). Further, while many microbiome studies have focused on the effects of dietary fiber, fat and carbohydrates (Conlon and Bird, 2014; Reese and Carmody, 2018 (Conlon and Bird, 2014; Reese and Carmody, 2018)), less is known about the effects of dietary protein and amino acids, even though 5-10% of dietary amino acids reach the colon where most gut bacterial metabolism occurs (Ahlman et al., 1993; Whitt and Demoss, 1975 (Ahlman et al., 1993; Whitt and Demoss, 1975)). Not wishing to be bound to a particular theory, it was hypothesized that gut microbial metabolism may function as a key intermediary linking dietary sulfur amino acids (Saa) to kidney function.

In humans, increasing dietary protein increases gut bacterial production of indole, an indoxyl sulfate precursor, and hydrogen sulfide (H₂S) (Magee et al., 2000; Poesen et al., 2015). The colon has the highest H₂S concentrations in the body, and there is a strong correlation between dietary protein intake and fecal H₂S levels (Linden, 2014; Magee et al., 2000). H₂S has diverse physiological functions, some of which are mediated by the post-translational modification S-sulfhydration (Mustafa et al., 2009; Paul and Snyder, 2012). For example, S-sulfhydration of the p65 subunit of nuclear factor κB (NF-κB) promotes its binding to the co-activator ribosomal protein S3 and induces anti-apoptotic gene expression (Sen et al., 2012). S-sulfhydration of the signaling phosphatase protein tyrosine phosphatase 1B (PTP1B) inhibits its activity, leading to reduced protein translation during endoplasmic reticulum stress (Krishnan et al., 2011), and S-sulfhydration of multiple Ca2+ TRP channels promotes self-renewal of mesenchymal stem cells (Liu et al., 2014). While a vast number of studies have been performed in mammalian systems (Paul and Snyder, 2015), the physiological roles of H₂S in regulating gut bacterial function within a host remain understudied. Additionally, whether there are bona fide opportunities to improve CKD by manipulating diet-microbiota interactions remain unclear.

To begin to determine the steps linking dietary SAA, gut microbial metabolism, and kidney function; a preclinical CKD model was employed. It was determined that mice on a low Saa diet had more severe kidney disease compared to those on a high Saa diet, and renal function was more impaired in specific pathogen-free (SPF) as compared to germ-free (GF) mice. To focus on microbial metabolism in relevant taxa, gut microbiome studies of CKD patients were analyzed and it a robust expansion of the family Enterobacteriaceae was discovered. The combination of a low Saa diet and the presence of E. coli, an Enterobacteriaceae family member, markedly exacerbated kidney damage in a CKD gnotobiotic model. Cecal sulfide levels varied with a high vs low Saa diet and proteomic profiling of the E. coli sulfhydrome revealed that tryptophanase (TnaA), an exo-enzyme that degrades tryptophan to ammonia, pyruvate and indole, was one of the most highly S-sulfhydrated proteins detected. Utilizing in vitro and in vivo experiments, it was found that S-sulfhydration inhibits TnaA, and use of E. coli mutants in gnotobiotic models supported that serum indoxyl sulfate and renal function were dependent on TnaA S-sulfhydration. Overall, this work uncovers a diet-microbe-host interaction centered on dietary amino acids as mediators of a microbial post-translational modification that may be clinically relevant for CKD patients.

### Results:

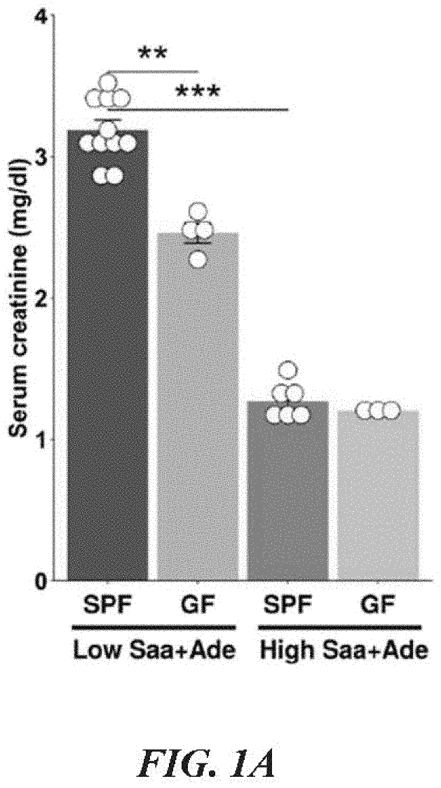

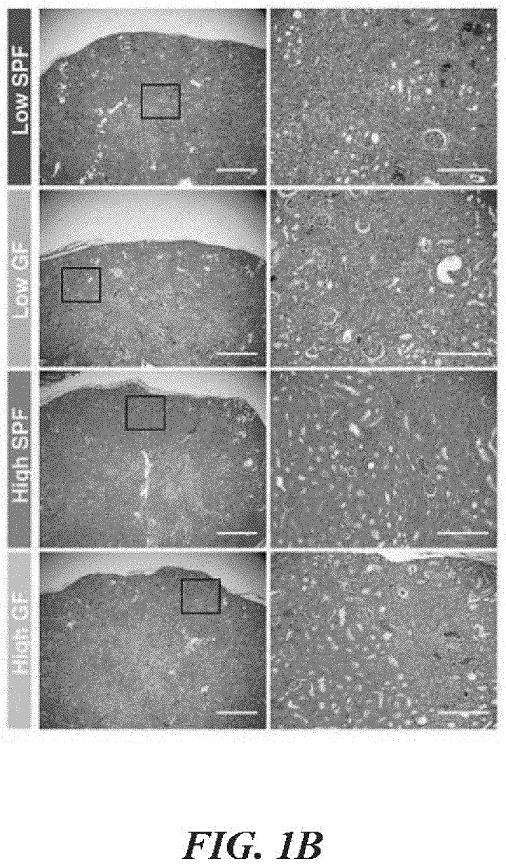

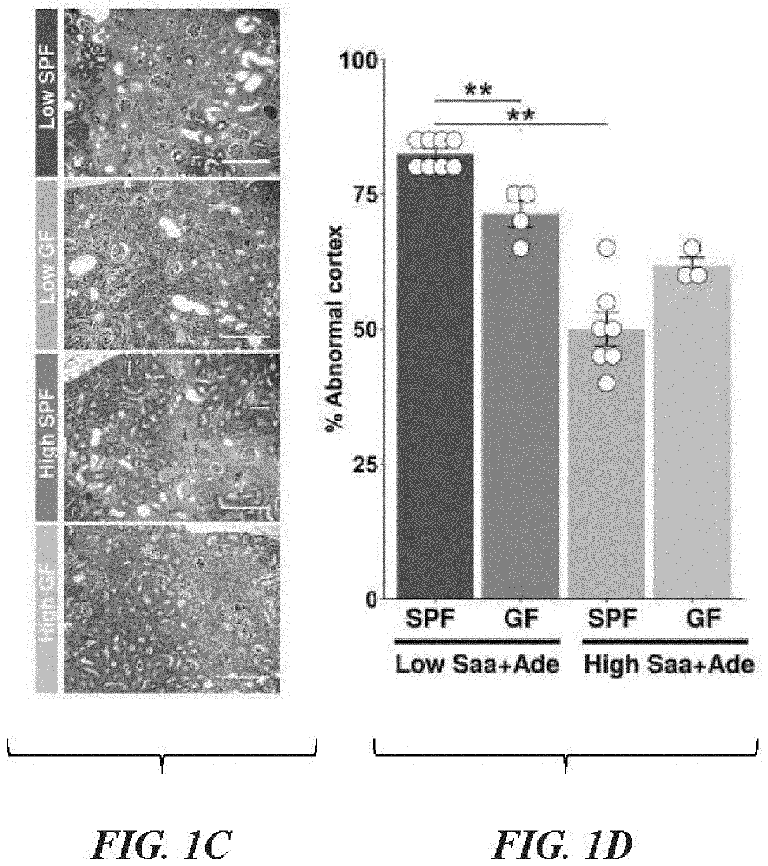

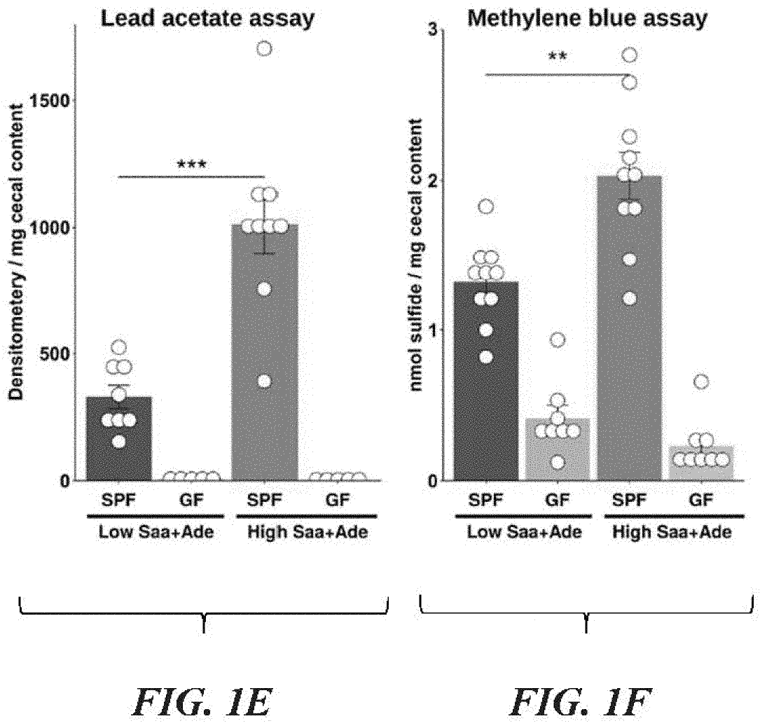

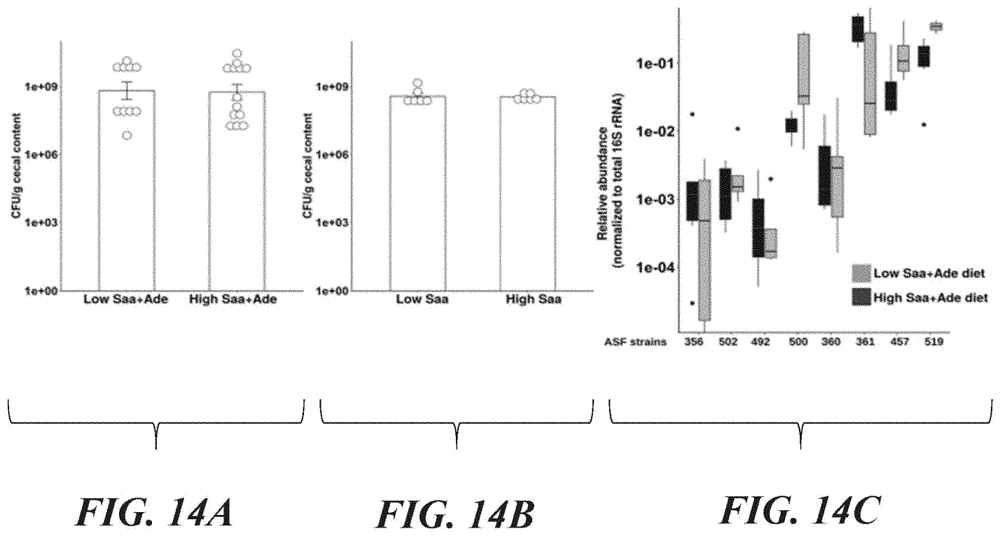

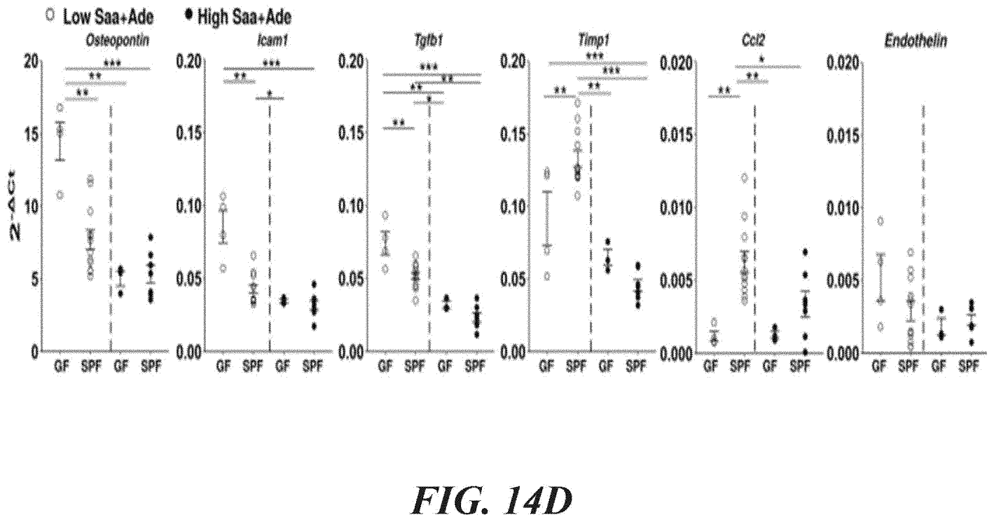

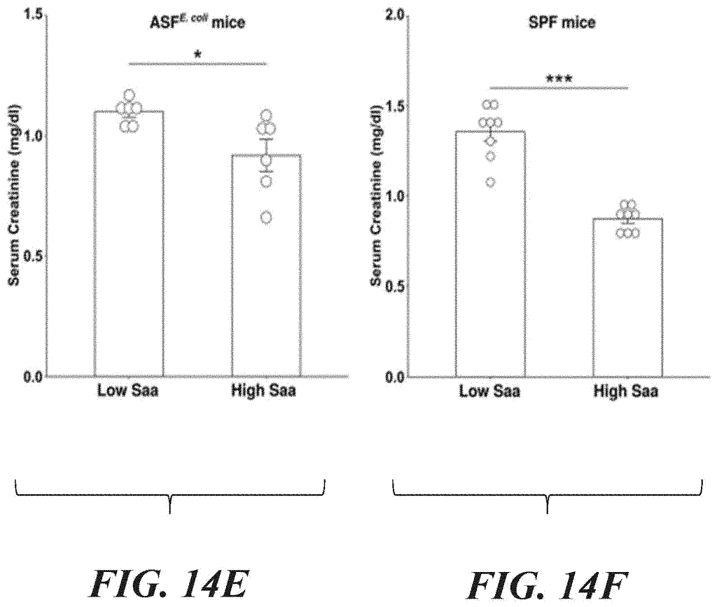

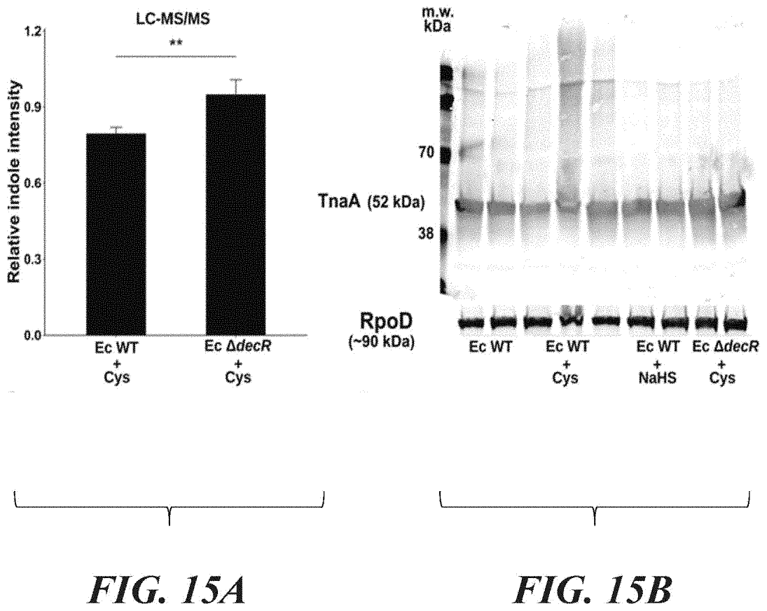

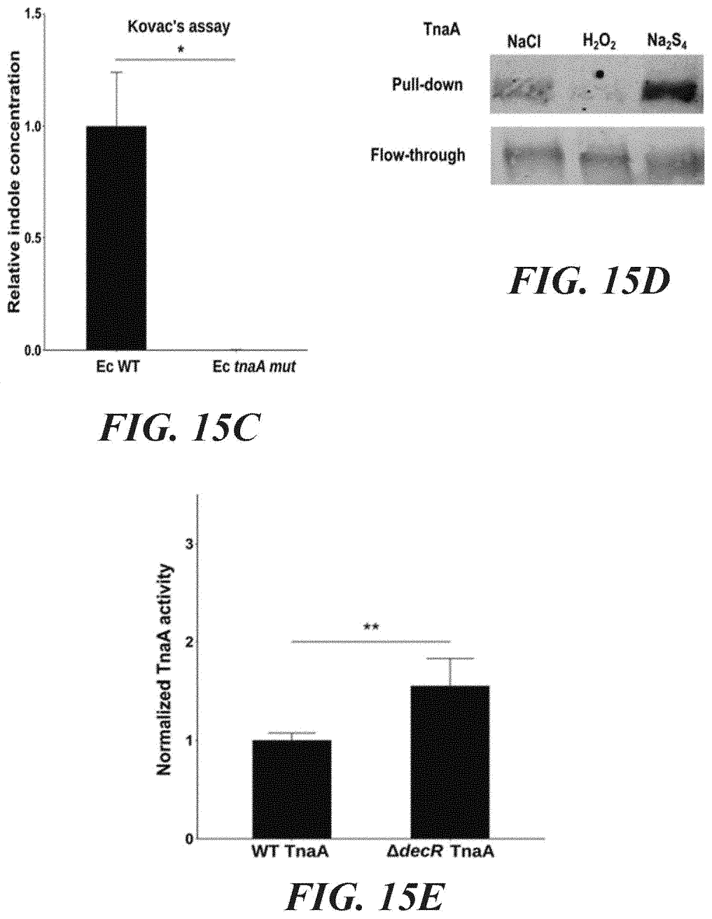

Dietary Saa Affect Kidney Function and Cecal Sulfide Levels in a Diet- and Microbiota-Dependent Manner: To begin to address the role of gut microbial metabolism and Saa in renal failure, a mouse model of CKD was employed that is driven by elevated dietary adenine (Jia et al., 2013). Isocaloric diets were formulated to represent edge cases of mouse Saa consumption, i.e. diets with low versus high amounts of methionine and cysteine (see TABLE 1 for the diet formulations) based on the literature (Elshorbagy et al., 2012; Paul et al., 2014). The lower Saa diet contains sufficient methionine to avoid the effects of methionine restriction (Cooke et al., 2018). Conventionally reared, specific-pathogen-free (SPF) mice on a low Saa+adenine (Saa+Ade) diet had significantly increased serum creatinine levels compared to mice on high Saa+Ade (mean 3.189 v 1.272, p<0.001) (FIG. 1A), as well as histology notable for worse tubular dilatation and drop-out, tubulitis with pen-tubular fibrosis, and cortical crystal deposition (FIG. 1B-1C). The low Saa+Ade diet also exacerbated the extent and severity of the renal parenchymal tubulointerstitial injury (FIG. 1B-1D). To determine the extent to which the Saa effects were dependent upon the gut microbiota, he Saa+Ade diets were fed to gnotobiotically-reared, germ-free (GF) mice. Serum creatinine (mean 1.76 vs 3.189 mg/dL, p<0.01) and kidney damage were markedly reduced in the GF mice as compared to SPF mice on the low Saa+Ade diet, while there were similar phenotypes in the GF and SPF mice fed the high Saa+Ade diet (FIG. 1A-1D). Given the extent of renal injury observed in GF mice on the low Saa+Ade diet, although less than SPF mice, we examined the expression of a select panel of host genes implicated in CKD pathogenesis in both humans and the mouse adenine model (12). Spp1 (Osteopontin), Tgfb1, and Icam1 were elevated in GF mice far more so than in SPF mice on the low Saa+Ade diet (FIG. 14D). In contrast, Ccl2 and Timp1 were far more elevated in SPF mice on the low Saa+Ade diet (FIG. 14D). These data suggest that the microbiota may buffer expression of some host genes while stimulating expressions of others via effects on diverse cell host populations, inclusive of immune, epithelial and stromal compartment cells, influencing renal injury susceptibility.

Overall, it was observed that a low Saa diet exacerbated the phenotypes observed with the adenine diet and the presence of gut microbiota further magnified these effects.

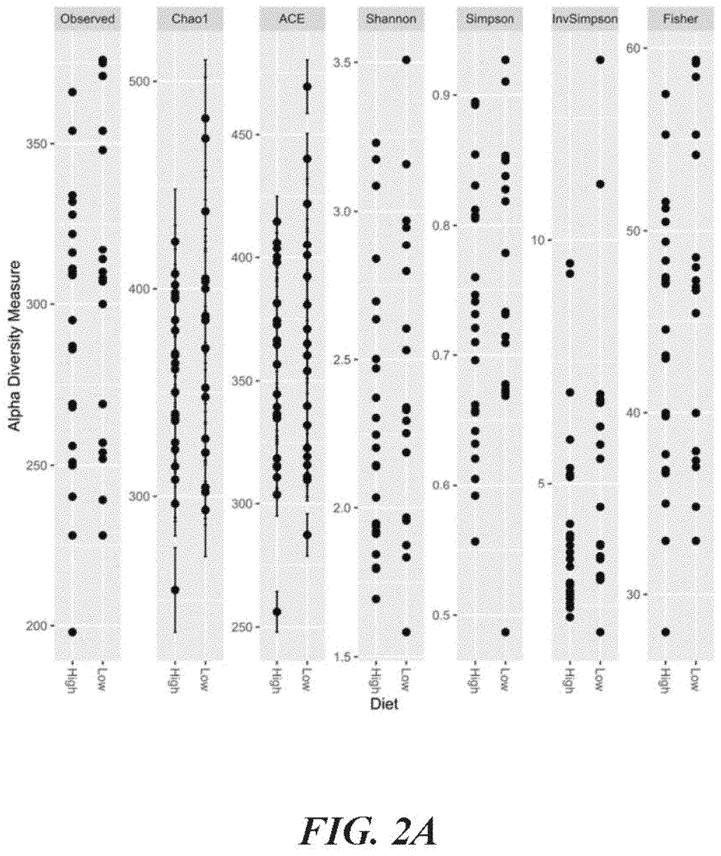

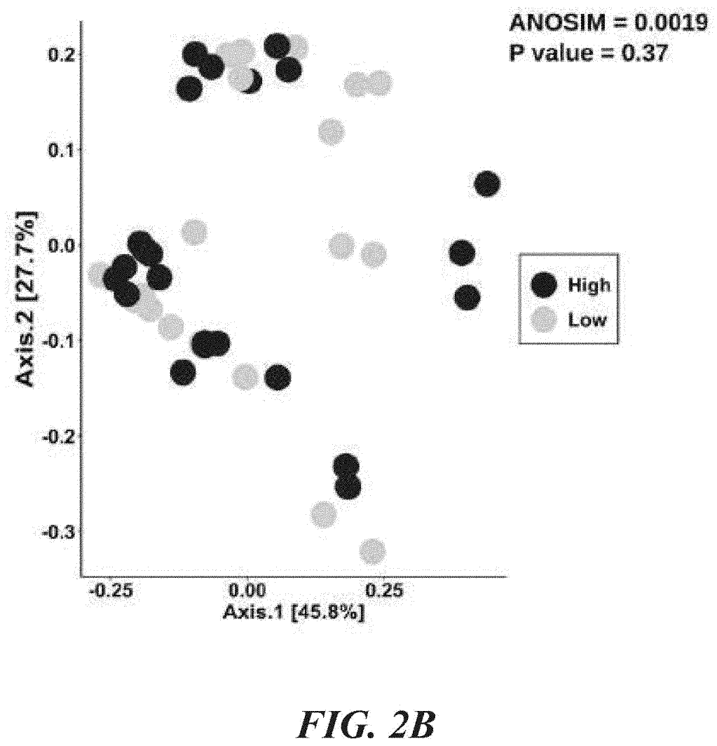

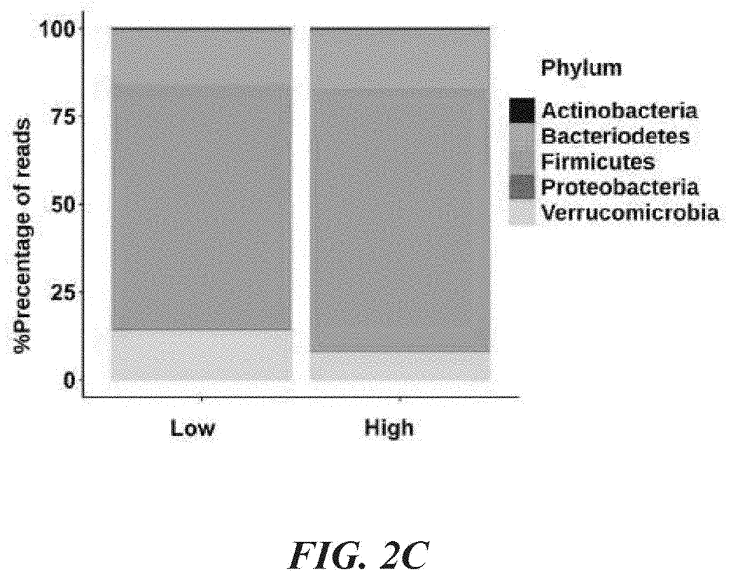

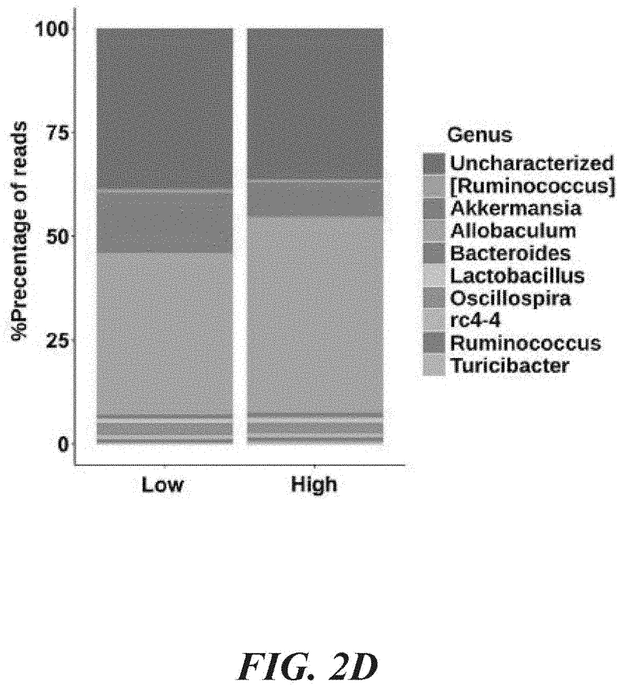

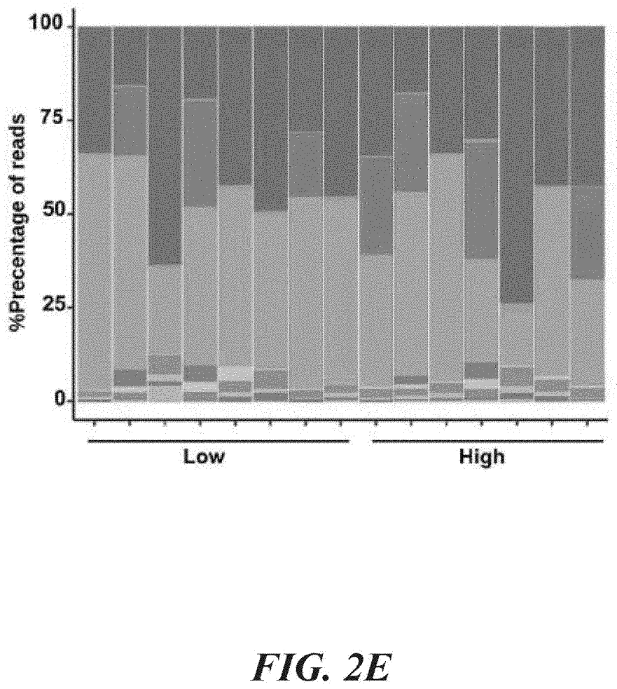

A plausible link between dietary Saa and gut bacteria is microbial metabolism of cysteine to H₂S. The cecal sulfide levels were measured from GF and SPF mice fed low versus high Saa diets using both the lead acetate and methylene blue sulfide detection assays (Hine and Mitchell, 2017). As expected, SPF mice on the high Saa diet had higher cecal sulfide levels (mean 3.1-fold higher, p<0.001, lead acetate assay; mean 1.5-fold higher, p<0.01, methylene blue assay; two-way ANOVA with Tukey's post-hoc test for both analyses) than those on the low Saa diet (FIG. 1E-1F). GF mouse ceca had significantly less sulfide than SPF mice, regardless of Saa diet (FIG. 1E-1F). No significant differences were observed in the taxonomic abundances of the gut microbiota between SPF mice on the low vs high Saa diets using 16S rRNA gene amplicon surveys (FIG. 2A-2E), supporting that the effect on cecal sulfide in healthy mice may be mediated by altering microbial function, rather than microbiota structure. Given these findings and with the goal of more effectively modeling such gut microbial activity shifts that could occur in humans with CKD, patient gut microbiota profiling studies were carried out to identify taxa enriched in CKD patients as compared to healthy individuals.

### Enrichment of Enterobacteriaceae in CKD Patient Gut Microbiota:

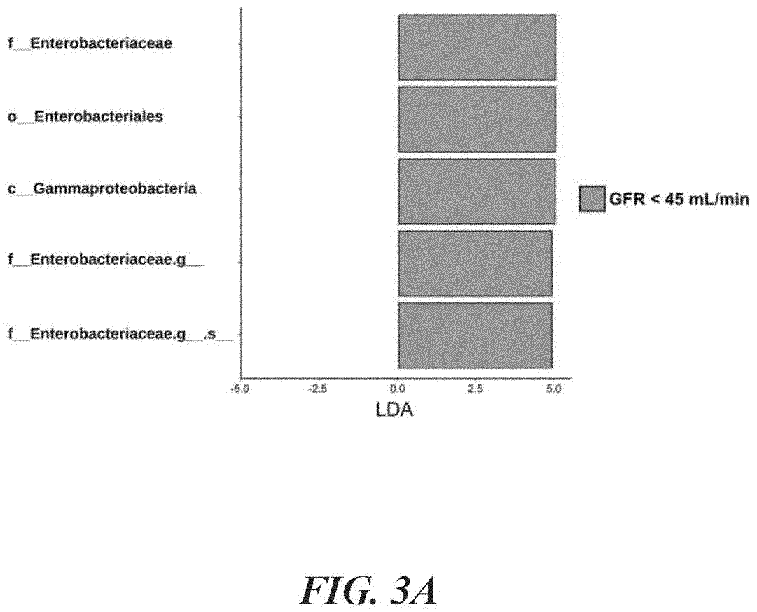

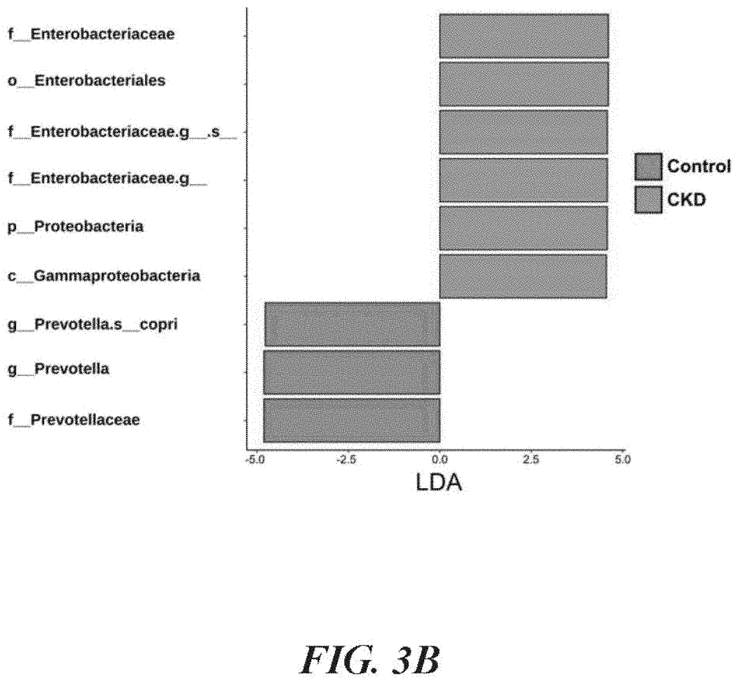

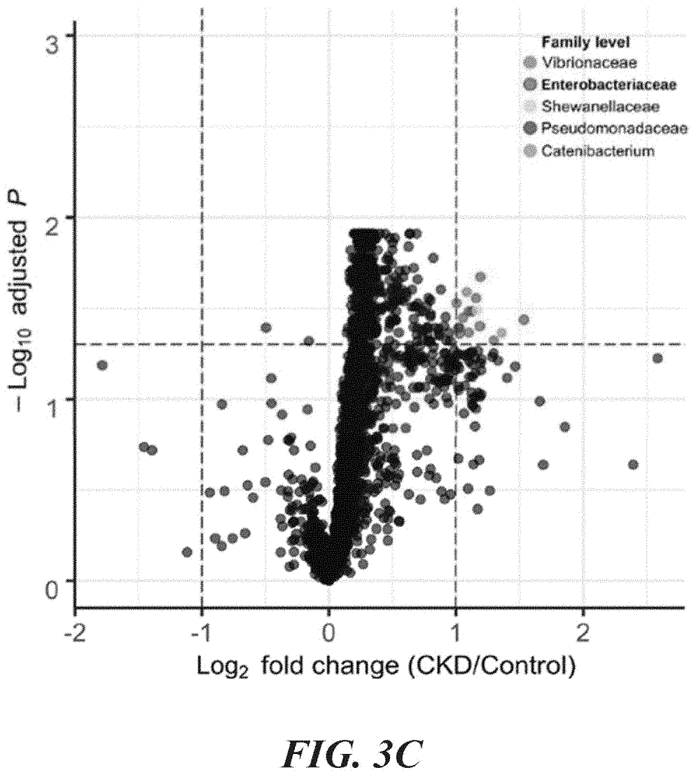

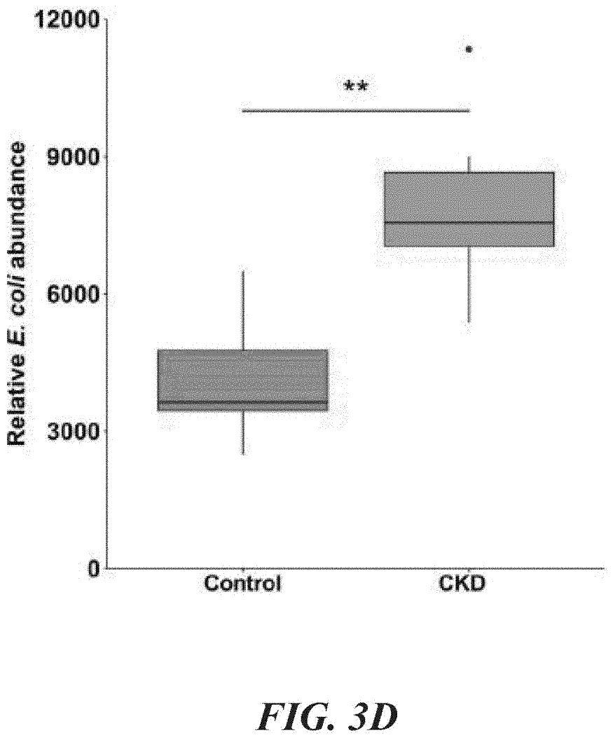

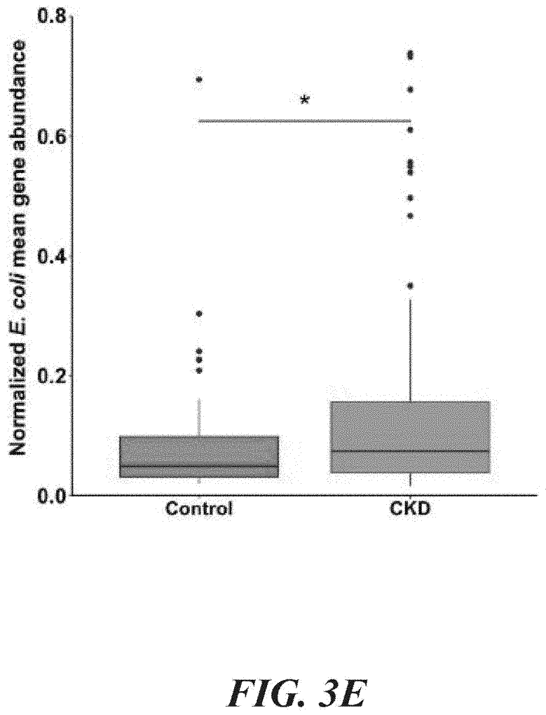

An early fecal culture-based study suggested that Escherichia coli, a typical gut Enterobacteriaceae member, are higher (CFU/gm stool) in samples from CKD patients compared to healthy controls (Fukuuchi et al., 2002). However, several more recent comprehensive studies, all of which determined that CKD patients have a distinct fecal microbiota compared with non-CKD controls (Li et al., 2019; Lun et al., 2019; Vaziri et al., 2013; Xu et al., 2017), observed different bacterial taxa that were altered in CKD patients across these individual studies. Data sets of fecal 16S rRNA gene amplicon datasets were analyzed. Enforcing stringent statistical cutoffs (LDA>4 for LEfSe analyses and fold change >2 for the PhyloChip analysis) revealed a clear and robust signal of Enterobacteriaceae enrichment in CKD patients (FIG. 3A-3C). Although the 16S rRNA gene amplicon analyses did not offer sufficient resolution for species level E. coli identification, the PhyloChip analysis showed a significant increase in the combined mean abundance of seven E. coli strains measured in fecal samples of end-stage renal disease (ESRD) patients compared to control subjects (FIG. 3D). Further analysis of the PTRI whole genome shotgun sequencing dataset strengthened this finding, as it was discovered that there was a higher normalized E. coli mean gene abundance in CKD patient samples compared to non-CKD controls (FIG. 3E). Thus, CKD patients have elevated Enterobacteriaceae abundance raising the possibility that this family, or E. coli in particular, could function in the development or progression of CKD.

E. coli Colonization of ASF Mice Exacerbates Kidney Failure in a CKD Model

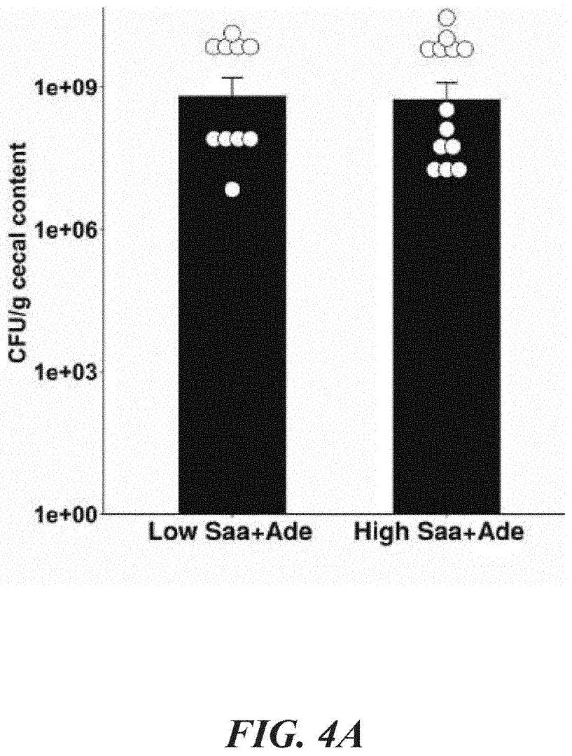

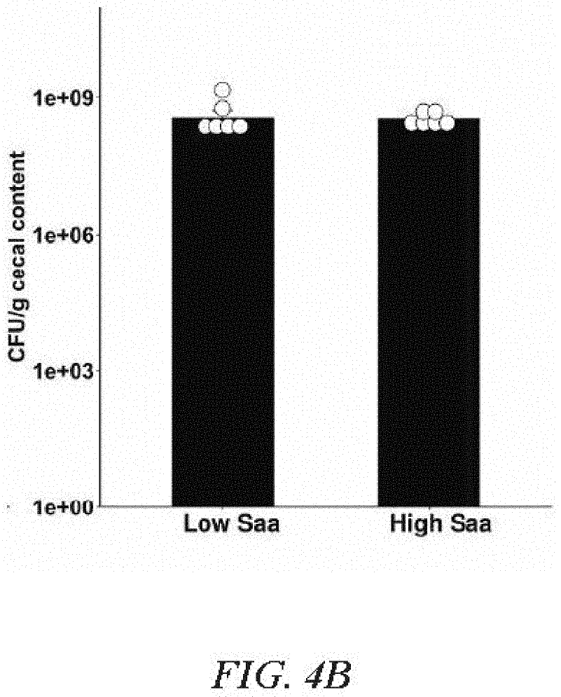

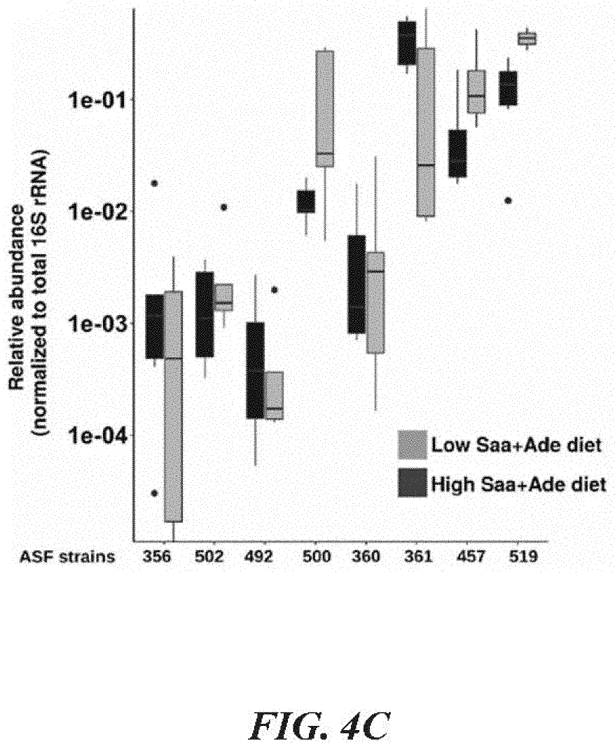

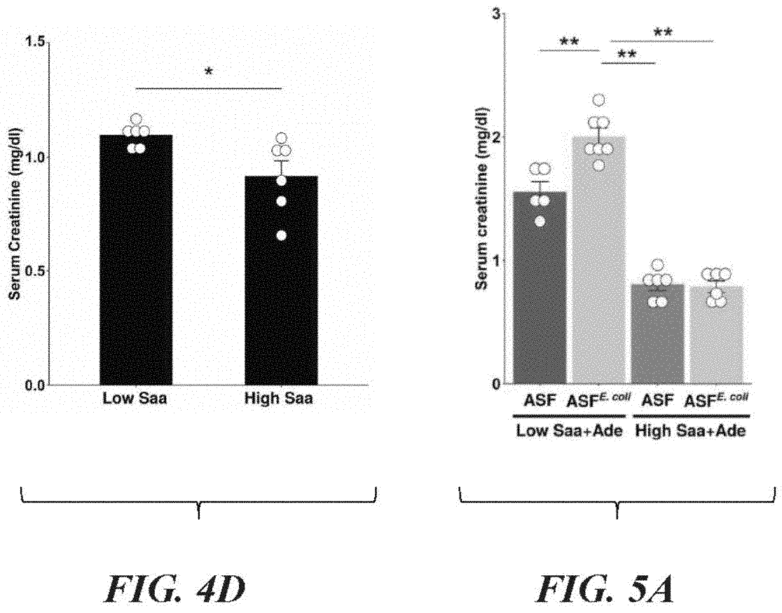

Given the findings from the re-analysis of human CKD gut microbiota studies and both the genetic tractability and relatively well-characterized proteome of E. coli, the effects of E. coli in the adenine-driven CKD model were analyzed. Since mice obtained from Jackson Laboratory harbor very few if any Enterobacteriaceae members (FIG. 2A-2E) (Rosshart et al., 2019) and given the need to carry out a detailed study of gut microbial activity in a reproducible model system in response to diet, gnotobiotically-reared mice colonized with the altered Schaedler Flora (ASF) were used, a simplified microbial community consisting of 8 bacterial species, none of which are related to Enterobacteriaceae or its phylum Proteobacteria (Brand et al., 2015) as the basal community. The ASF mice were employed, rather than mono-colonized mice, because ASF mice are more physiologically similar to SPF mice (Brand et al., 2015). The studies then generated gnotobiotically-reared ASF mice colonized with the genetically tractable and well-characterized E. coli K-12, referred to herein as ASF^{E.coli}. E. coli colonization was similar on low and high Saa and Saa+Ade diets (FIGS. 4A-4B), and in these studies it changes in the relative abundance of ASF members were not observed (FIG. 4C).

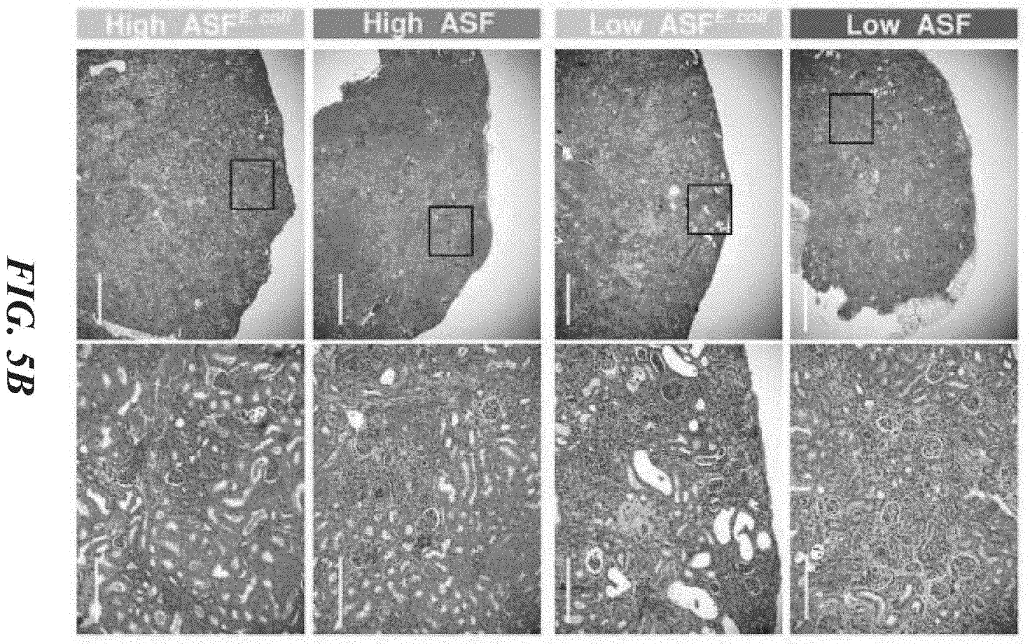

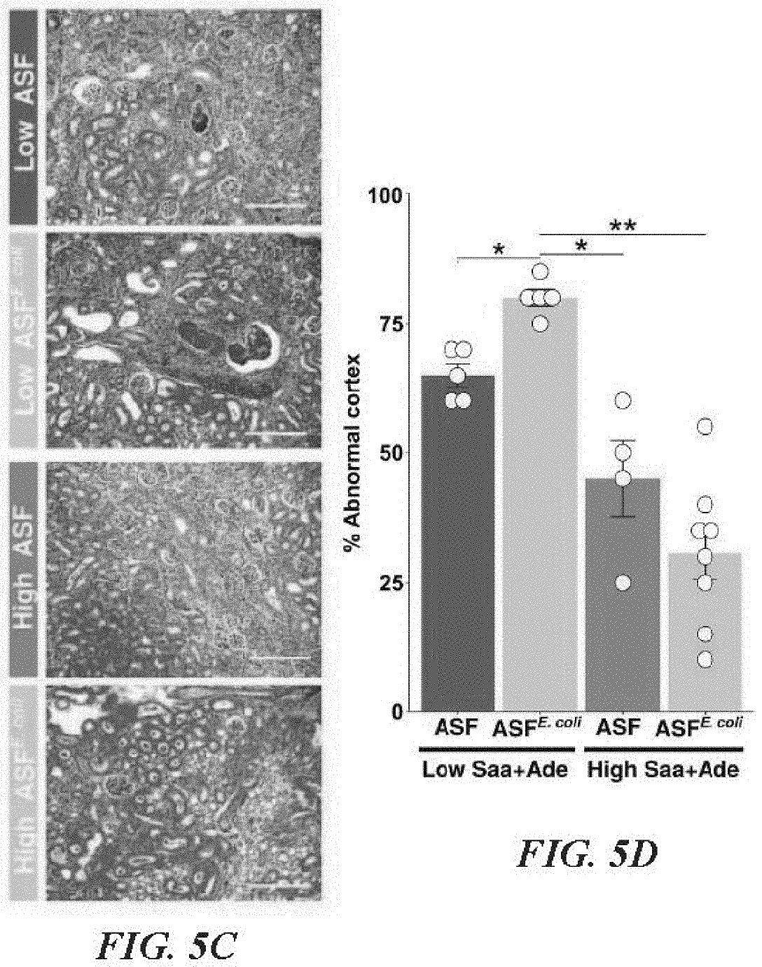

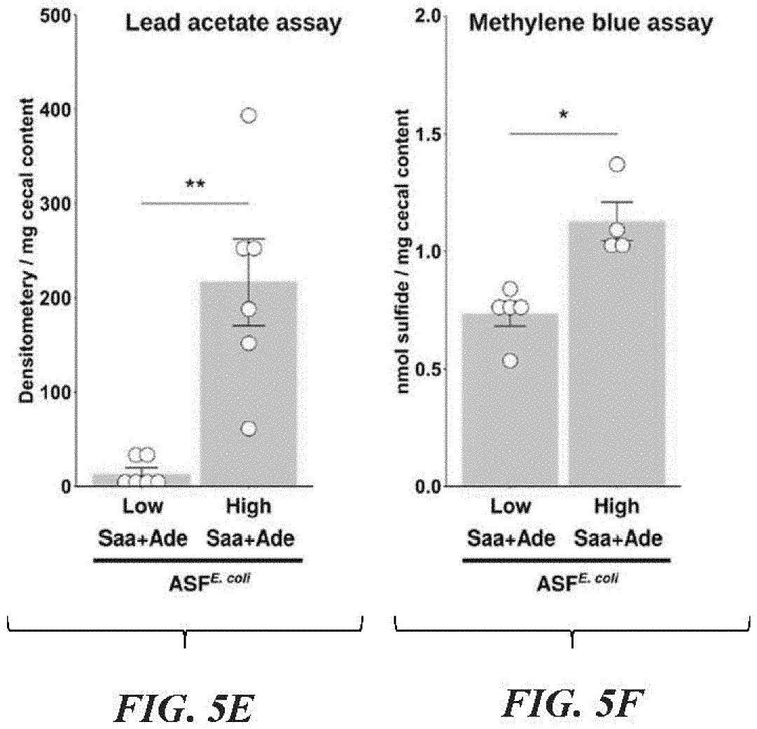

On the low Saa+Ade diet, ASF E. coli mice had higher serum creatinine (mean 2.25 vs 1.55 mg/dL, p<0.01, two-way ANOVA with Tukey's post-hoc test) and more extensive tubulitis, tubular atrophy and drop-out, peritubular fibrosis, and cortical crystals with increased parenchymal involvement than ASF mice on the low Saa+Ade diet (FIG. 5A-5C). In contrast, ASF E. coli and ASF mice on the high Saa+Ade diet had similar serum creatinine levels and milder kidney pathology compared with their littermates on the low Saa+Ade diet (FIG. 5A-5C). As with the SPF mice, it was found that higher cecal sulfide levels in ASF E. coli mice on the high versus low Saa+Ade diet (mean 16.5-fold higher, p<0.01, lead acetate assay; mean 1.5-fold higher, p<0.05 methylene blue assay; Mann-Whitney test for both analyses) (FIG. 5D-5E).

To determine if changes to renal function would occur in these models in the absence of the adenine insult, the studies examined creatinine levels in ASF E. coli mice on the low versus high Saa diet. The low Saa diet and E. coli were sufficient to increase serum creatinine levels in mice on the high Saa diet and no overt histologic abnormalities were present (FIG. 4A, FIG. 14E). Similar results were obtained with SPF mice on the Saa diets (FIG. 14F). Overall, these results support that E. coli interacts with dietary Saa to modulate kidney function.

Analysis of the E. coli S-Sulfhydrome Reveals S-Sulfhydration of Tryptophanase

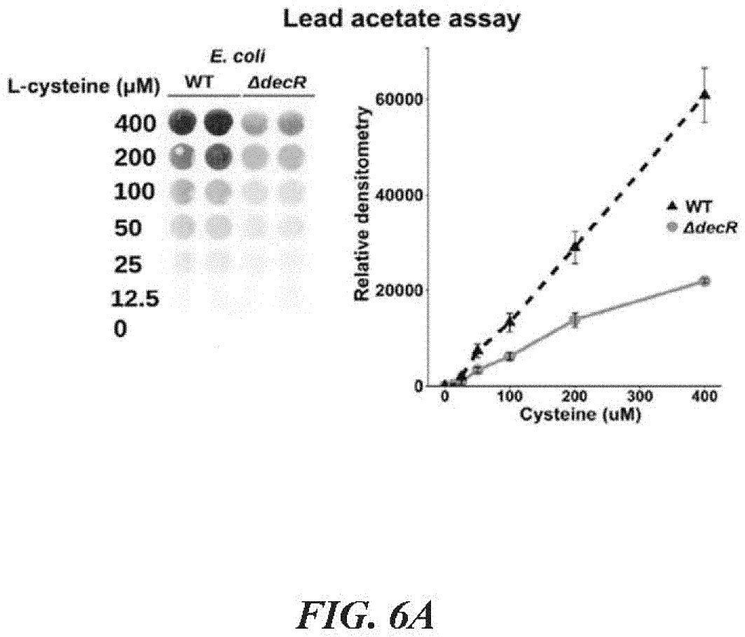

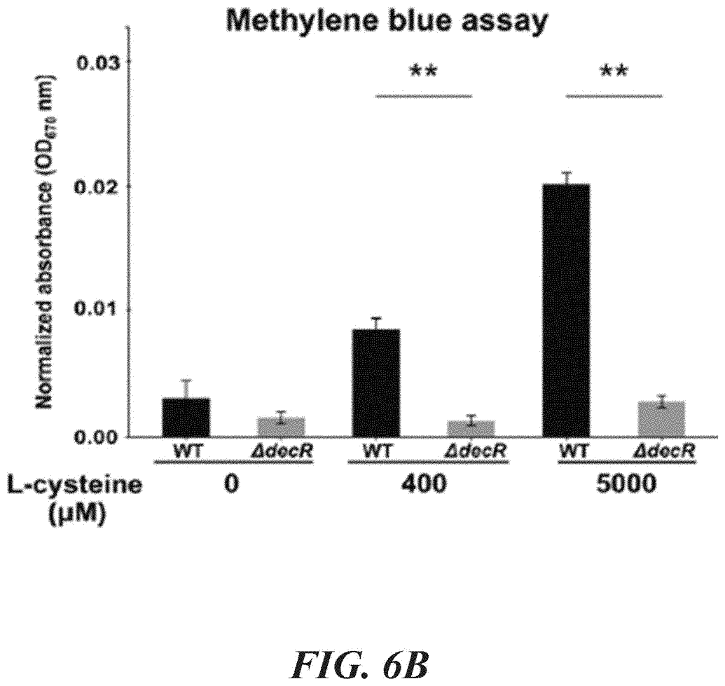

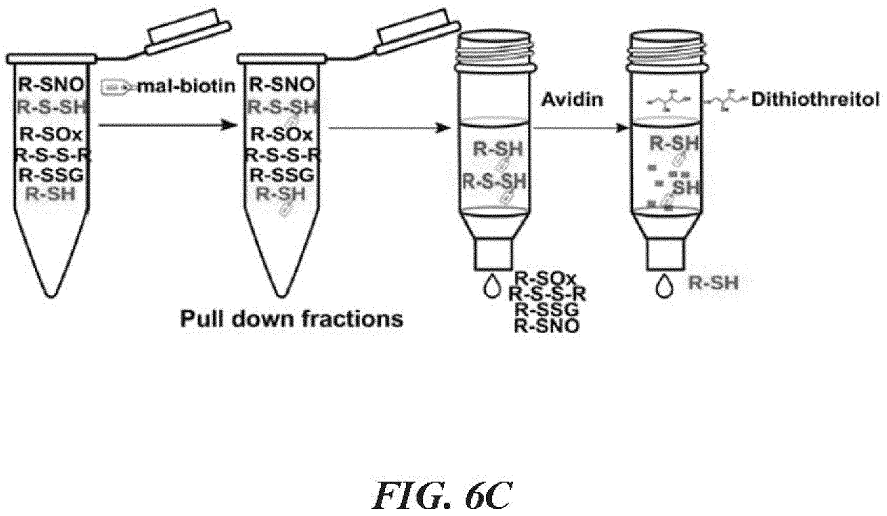

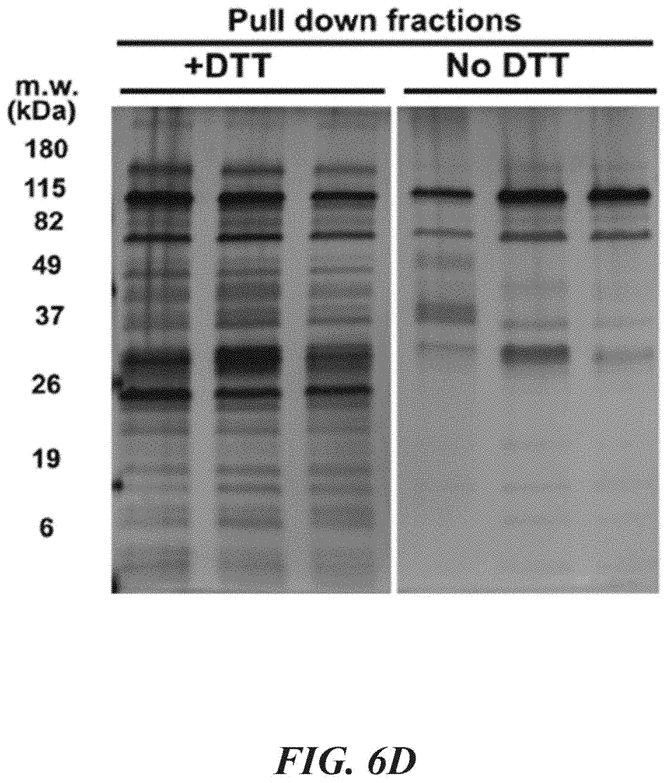

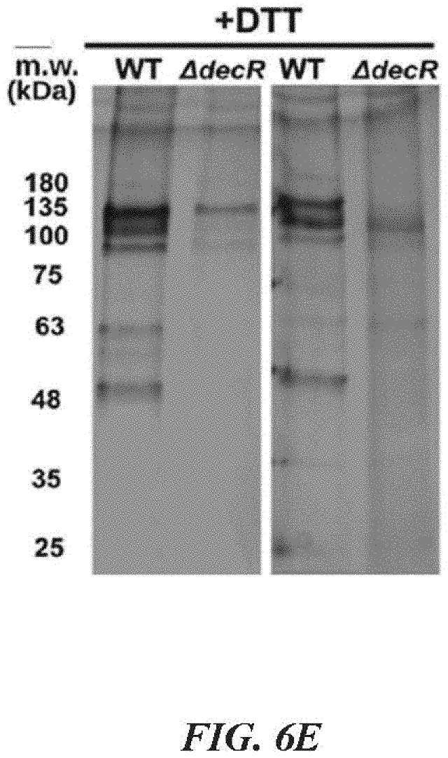

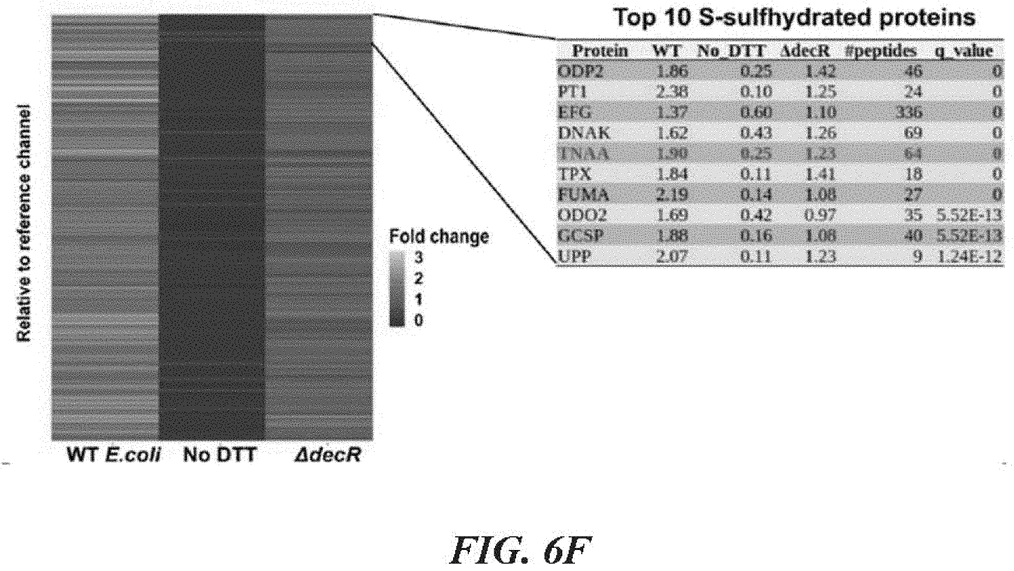

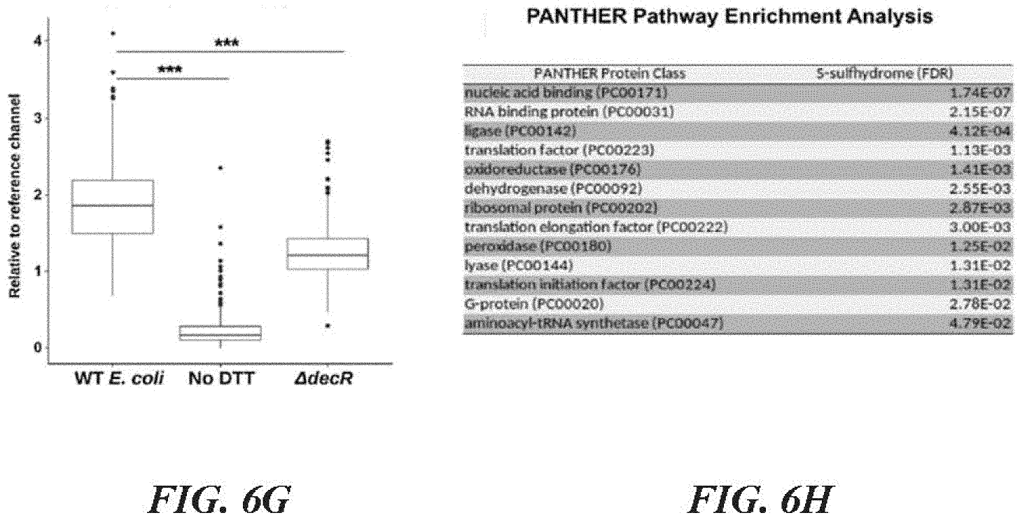

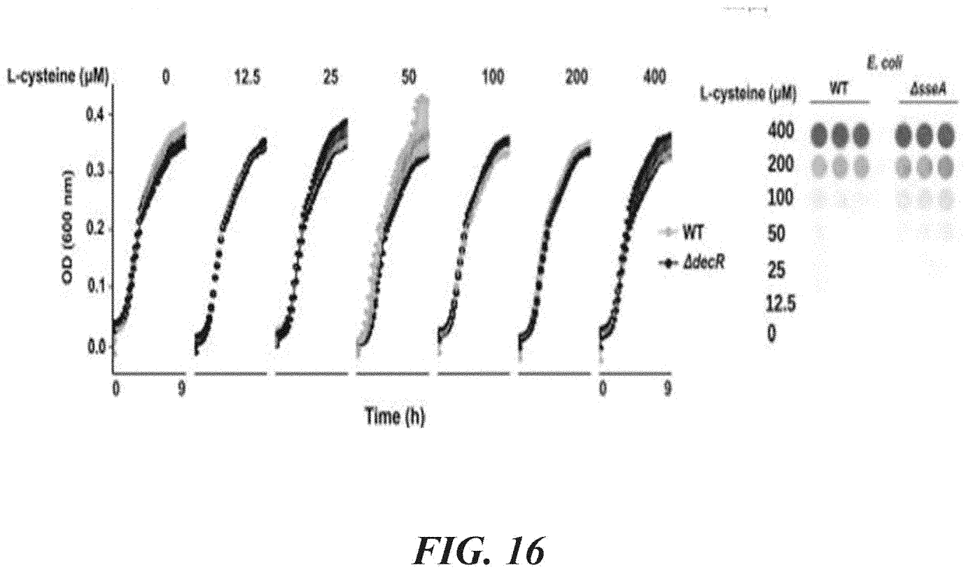

Given these observations regarding cecal H₂S in SPF and ASF E. coli mice on the Saa diets and the vast literature on how H₂S can post-translationally modify mammalian proteins leading to a range of physiologic effects, the studies delved into examining the effects of H₂S on E. coli. In lead acetate sulfide detection assays, E. coli produced sulfide from cysteine in a dose-dependent manner, when grown aerobically or anaerobically, without any effects on growth (FIG. 6A and FIG. 4A-4B). To serve as a control for how endogenous H₂S production affects E. coli physiology, an isogenic strain harboring a deletion of decR was generated, which encodes a transcriptional activator of the cystine de-sulfhydrase yhaOM, the main contributor to cysteine-derived sulfide production in E. coli (Shimada et al., 2016). As expected, decR deletion resulted in significant reduction of sulfide production in the lead acetate assay, with no effect on growth kinetics (FIGS. 6A-6B and FIGS. 7A-7C, FIG. 16).

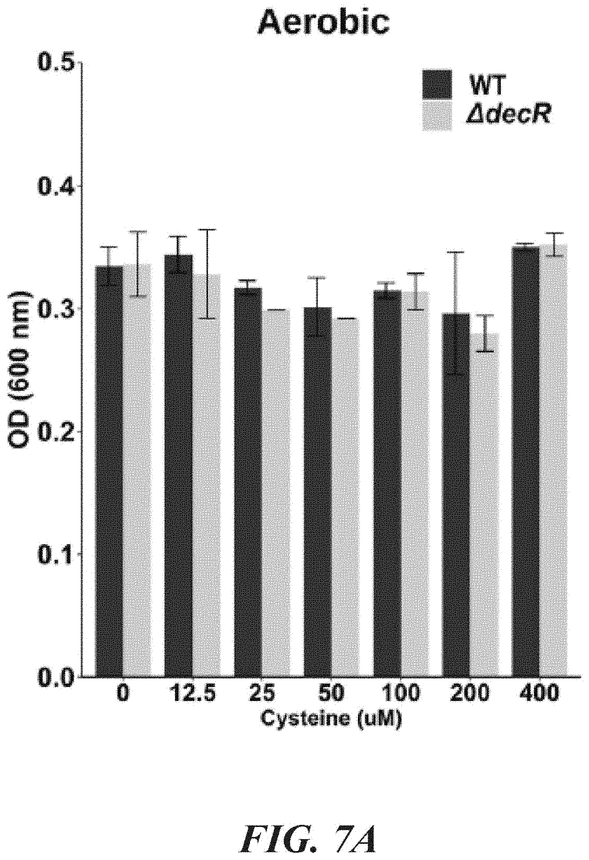

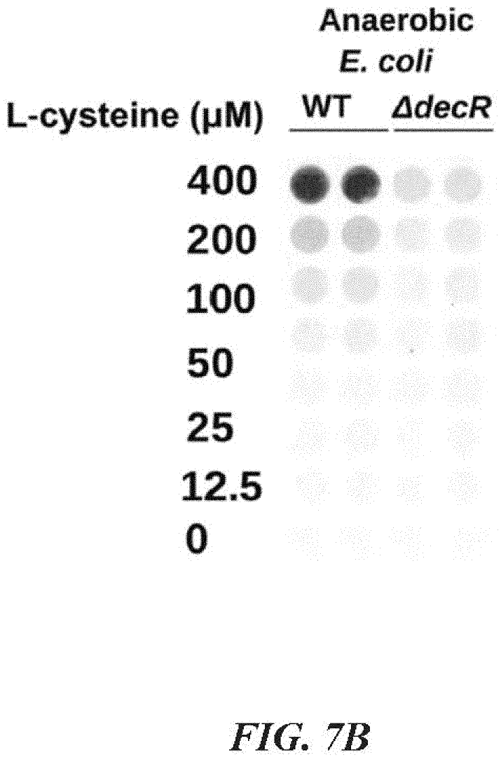

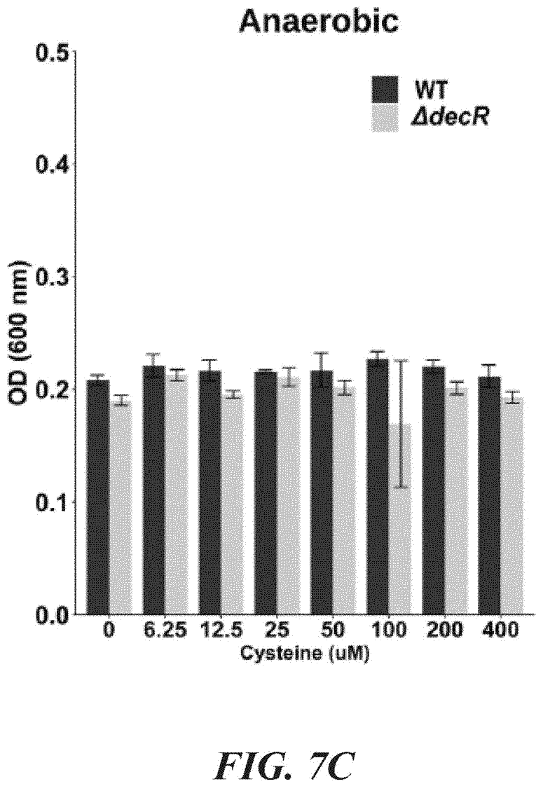

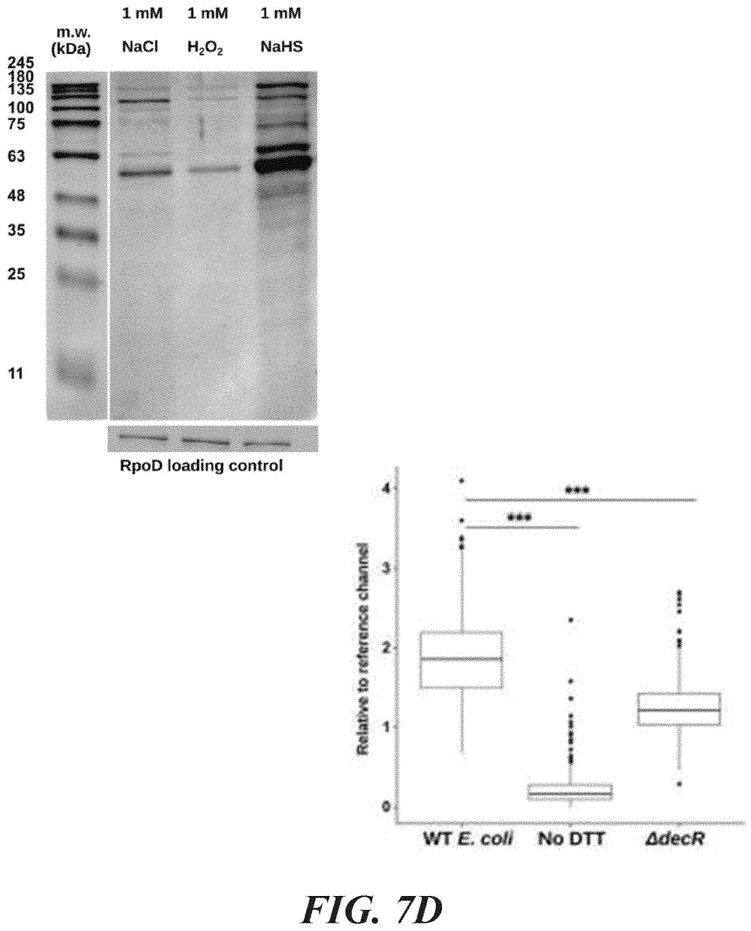

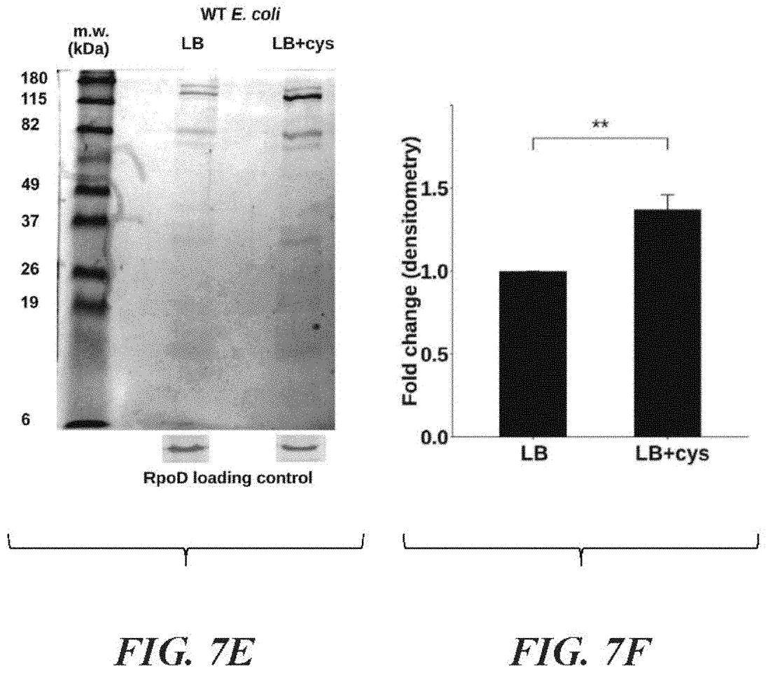

The major molecular mechanism by which sulfide exerts its effects is through generation of polysulfides that modify cysteine residues, resulting in S-sulfhydration (Mishanina et al., 2015). To identify E. coli proteins that are S-sulfhydrated (R—S—S), a pull-down method that leverages maleimide binding to free thiols was adapted, resulting in thioester bonds, and the ability of dithiothreitol (DTT) to break disulfide bonds but not thioester bonds, to specifically enrich for S-sulfhydrated proteins (FIG. 6C) (Gao et al., 2015). A robust enrichment of S-sulfhydrated proteins in DTT-eluted samples was observed using this method on WT E. coli lysates grown in media supplemented with cysteine (FIG. 6D). Several control experiments were performed to validate the specificity of the pull-down assay and found that treating bacterial lysates with H₂O₂, and hence oxidizing free thiols, reduced the detection of S-sulfhydration proteins (FIG. 7D). In contrast, treatment with sodium hydrosulfide (NaHS), a fast-reacting sulfide donor, induced higher S-sulfhydration levels in bacterial lysates (FIG. 7D). A higher level of S-sulfhydration in E. coli lysates grown in media supplemented with cysteine was detected as compared to E. coli grown in LB alone (FIG. 7E). In contrast, lysates of ΔdecR bacteria, which produce less H₂S, grown in cysteine-supplemented LB broth had lower S-sulfhydration than WT E. coli (FIG. 6E).

Having validated the enrichment method, the next studies sought to characterize the E. coli sulfhydrome using quantitative tandem mass tag (TMT) LC-MS3 analysis. This analysis revealed that most identified proteins were indeed S-sulfhydrated, as they were enriched in E. coli lysates that were eluted with DTT versus the same lysate samples that were not treated with DTT (FIG. 6F-6G). Furthermore, most detected S-sulfhydrated proteins were enriched in WT vs ΔdecR E. coli, as expected from the strains' differential ability to produce sulfide from cysteine. Ranking of the S-sulfhydrated proteins by their q values (DTT versus non-DTT) revealed the top 10 most abundant S-sulfhydrated proteins (FIG. 6F). While some of these proteins are highly expressed during logarithmic bacterial growth, and thus are expected to be highly abundant, others like tryptophanase (TnaA) were over-represented. A more readily quantifiable representation of the data is presented in a boxplot chart (FIG. 6G). Overall, the quantitative proteomics analysis identified 212 proteins as S-sulfhydrated with high confidence (TABLE 2) and hyper-geometric distribution analysis revealed thirteen cellular pathways enriched with S-sulfhydrated proteins, several of which are related to protein translation (FIG. 611).

TABLE 2
S-SULFHYDRATED PEPTIDES AND S-SULFHYDRATED PROTEINS

| | | | | | No | | | q value | q value |
|---|---|---|---|---|---|---|---|---|---|
| | | No | | WT | DTT | ΔdecR | Num of | WT vs | WT vs |
| Accession | WT | DTT | ΔdecR | SD | SD | SD | peptides | No DTT | ΔdecR |
| sp\|P06959\|ODP2_ | 1.86 | 0.25 | 1.42 | 0.51 | 0.09 | 0.34 | 46.00 | 0.00 | 0.00 |
| ECOLI | | | | | | | | | |
| sp_P08839\|PT1_ | 2.38 | 0.10 | 1.25 | 0.67 | 0.07 | 0.41 | 24.00 | 0.00 | 0.00 |
| ECOLI | | | | | | | | | |
| sp\|P0A6M8\|EFG_ | 1.37 | 0.60 | 1.10 | 0.52 | 0.26 | 0.51 | 336.00 | 0.00 | 0.00 |
| ECOLI | | | | | | | | | |
| sp\|P0A6Y8\|DNAK_ | 1.62 | 0.43 | 1.26 | 0.65 | 0.20 | 0.63 | 69.00 | 0.00 | 0.00 |
| ECOLI | | | | | | | | | |
| sp\|P0A853\|TNAA_ | 1.90 | 0.25 | 1.23 | 0.79 | 0.14 | 0.52 | 64.00 | 0.00 | 0.00 |
| ECOLI | | | | | | | | | |
| sp\|P0A862\|TPX_ | 1.84 | 0.11 | 1.41 | 0.47 | 0.08 | 0.39 | 18.00 | 0.00 | 0.00 |
| ECOLI | | | | | | | | | |
| sp\|P0AC33\|FUMA_ | 2.19 | 0.14 | 1.08 | 0.98 | 0.11 | 0.34 | 27.00 | 0.00 | 0.00 |
| ECOLI | | | | | | | | | |
| sp\|P0AFG6\|ODO2_ | 1.69 | 0.42 | 0.97 | 0.42 | 0.14 | 0.34 | 35.00 | 0.00 | 0.00 |
| ECOLI | | | | | | | | | |
| sp\|P33195\|GCSP_ | 1.88 | 0.16 | 1.08 | 0.57 | 0.10 | 0.41 | 40.00 | 0.00 | 0.00 |
| ECOLI | | | | | | | | | |
| sp\|P0A8F0\|UPP_ | 2.07 | 0.11 | 1.23 | 0.36 | 0.06 | 0.17 | 9.00 | 0.00 | 0.00 |
| ECOLI | | | | | | | | | |
| sp\|P0CE47\|EFTU1_ | 2.35 | 0.10 | 1.53 | 1.03 | 0.11 | 2.31 | 72.00 | 0.00 | 0.01 |
| ECOLI; | | | | | | | | | |
| sp\|P0CE48\|EFTU2_ | | | | | | | | | |
| ECOLI | | | | | | | | | |
| sp\|P07012\|RF2_ | 2.16 | 0.13 | 1.05 | 0.34 | 0.08 | 0.34 | 11.00 | 0.00 | 0.00 |
| ECOLI | | | | | | | | | |
| sp\|P0A6Z1\|HSCA_ | 2.25 | 0.15 | 1.10 | 0.41 | 0.04 | 0.34 | 12.00 | 0.00 | 0.00 |
| ECOLI | | | | | | | | | |
| sp\|P0A7R5\|RS10_ | 2.97 | 0.17 | 0.88 | 0.44 | 0.07 | 0.18 | 8.00 | 0.00 | 0.00 |
| ECOLI | | | | | | | | | |
| sp\|P0ADG7\|IMDH_ | 1.53 | 0.16 | 1.08 | 0.20 | 0.12 | 0.24 | 12.00 | 0.00 | 0.00 |
| ECOLI | | | | | | | | | |
| sp\|P32132\|TYPA_ | 2.03 | 0.16 | 1.20 | 0.30 | 0.13 | 0.41 | 11.00 | 0.00 | 0.00 |
| ECOLI | | | | | | | | | |
| sp\|P0A9W3\|ETTA_ | 1.99 | 0.16 | 1.05 | 0.46 | 0.10 | 0.16 | 14.00 | 0.00 | 0.00 |
| ECOLI | | | | | | | | | |
| sp\|P14407\|FUMB_ | 1.87 | 0.15 | 1.14 | 0.30 | 0.09 | 0.23 | 14.00 | 0.00 | 0.00 |
| ECOLI; | | | | | | | | | |
| sp\|P0AC33\|FUMA_ | | | | | | | | | |
| ECOLI | | | | | | | | | |
| sp\|P33602\|NUOG_ | 1.92 | 0.22 | 1.29 | 0.51 | 0.23 | 0.26 | 14.00 | 0.00 | 0.00 |
| ECOLI | | | | | | | | | |
| sp\|P0A7D4\|PURA_ | 1.91 | 0.16 | 1.50 | 0.33 | 0.18 | 0.54 | 16.00 | 0.00 | 0.02 |
| ECOLI | | | | | | | | | |
| sp\|P0A9G6\|ACEA_ | 1.43 | 0.30 | 1.12 | 0.22 | 0.13 | 0.24 | 16.00 | 0.00 | 0.00 |
| ECOLI | | | | | | | | | |
| sp\|P0AG55\|RL6_ | 2.94 | 0.15 | 0.87 | 0.50 | 0.05 | 0.33 | 16.00 | 0.00 | 0.00 |
| ECOLI | | | | | | | | | |
| sp\|P00957\|SYA_ | 1.95 | 0.20 | 1.25 | 0.46 | 0.30 | 0.44 | 14.00 | 0.00 | 0.00 |
| ECOLI | | | | | | | | | |
| sp\|P0A836\|SUCC_ | 1.80 | 0.17 | 1.36 | 0.31 | 0.18 | 0.52 | 13.00 | 0.00 | 0.02 |
| ECOLI | | | | | | | | | |
| sp\|P0AAX8\|YBIS_ | 1.93 | 0.17 | 1.01 | 0.37 | 0.11 | 0.40 | 11.00 | 0.00 | 0.00 |
| ECOLI | | | | | | | | | |
| sp\|P08200\|IDH_ | 1.59 | 0.10 | 1.25 | 0.43 | 0.07 | 0.36 | 19.00 | 0.00 | 0.01 |
| ECOLI | | | | | | | | | |
| sp\|P0ADE8\|YGFZ_ | 1.85 | 0.15 | 1.43 | 0.24 | 0.10 | 0.28 | 19.00 | 0.00 | 0.00 |
| ECOLI | | | | | | | | | |
| sp\|P00968\|CARB_ | 1.79 | 0.17 | 1.52 | 0.64 | 0.22 | 0.33 | 16.00 | 0.00 | 0.23 |
| ECOLI | | | | | | | | | |
| sp\|P35340\|AHPF_ | 1.90 | 0.17 | 1.29 | 0.84 | 0.13 | 0.36 | 19.00 | 0.00 | 0.01 |
| ECOLI | | | | | | | | | |
| sp\|P0AB71\|ALF_ | 2.17 | 0.08 | 0.83 | 0.41 | 0.07 | 0.22 | 8.00 | 0.00 | 0.00 |
| ECOLI | | | | | | | | | |
| sp\|P0ABK5\|CYSK_ | 1.45 | 0.42 | 1.21 | 0.39 | 0.16 | 0.23 | 15.00 | 0.00 | 0.08 |
| ECOLI | | | | | | | | | |
| sp\|P0A850\|TIG_ | 1.60 | 0.12 | 1.66 | 0.50 | 0.13 | 0.50 | 15.00 | 0.00 | 0.94 |
| ECOLI | | | | | | | | | |
| sp\|P0A9B2\|G3P1_ | 1.49 | 0.24 | 1.13 | 0.27 | 0.07 | 0.15 | 8.00 | 0.00 | 0.00 |
| ECOLI | | | | | | | | | |
| sp\|P0AC41\|SDHA_ | 2.14 | 0.10 | 1.24 | 0.53 | 0.09 | 0.33 | 21.00 | 0.00 | 0.00 |
| ECOLI | | | | | | | | | |
| sp\|P64588\|YQJI_ | 3.36 | 0.11 | 0.29 | 2.04 | 0.11 | 0.21 | 21.00 | 0.00 | 0.00 |
| ECOLI | | | | | | | | | |
| sp\|P0A8A0\|YEBC_ | 2.11 | 0.18 | 0.94 | 0.09 | 0.13 | 0.16 | 5.00 | 0.00 | 0.00 |
| ECOLI | | | | | | | | | |
| sp\|P0ACF8\|HNS_ | 2.16 | 0.07 | 1.00 | 1.10 | 0.08 | 0.31 | 16.00 | 0.00 | 0.00 |
| ECOLI | | | | | | | | | |
| sp\|P0A6T3\|GAL1_ | 2.20 | 0.08 | 1.11 | 0.32 | 0.07 | 0.35 | 7.00 | 0.00 | 0.00 |
| ECOLI | | | | | | | | | |
| sp\|P21599\|KPYK2_ | 1.80 | 0.17 | 1.76 | 0.77 | 0.18 | 0.85 | 22.00 | 0.00 | 0.99 |
| ECOLI | | | | | | | | | |
| sp\|P0A8G6\|NQOR_ | 2.64 | 0.14 | 1.39 | 0.86 | 0.08 | 0.29 | 10.00 | 0.00 | 0.00 |
| ECOLI | | | | | | | | | |
| sp\|P0ACC3\|ERPA_ | 1.65 | 0.08 | 1.55 | 0.34 | 0.06 | 0.11 | 7.00 | 0.00 | 0.69 |
| ECOLI | | | | | | | | | |
| sp\|P09373\|PFLB_ | 1.91 | 0.07 | 1.48 | 0.65 | 0.10 | 0.57 | 34.00 | 0.00 | 0.00 |
| ECOLI | | | | | | | | | |
| sp\|P0A9Q1\|ARCA_ | 2.13 | 0.04 | 1.27 | 0.77 | 0.05 | 0.59 | 12.00 | 0.00 | 0.00 |
| ECOLI | | | | | | | | | |
| sp\|P36683\|ACNB_ | 2.56 | 0.17 | 1.04 | 0.93 | 0.07 | 0.30 | 31.00 | 0.00 | 0.00 |
| ECOLI | | | | | | | | | |
| sp\|P0AEI1\|MIAB_ | 2.13 | 0.10 | 0.90 | 0.54 | 0.08 | 0.44 | 9.00 | 0.00 | 0.00 |
| ECOLI | | | | | | | | | |
| sp\|P0ACD4\|ISCU_ | 2.11 | 0.14 | 1.15 | 0.77 | 0.17 | 0.48 | 12.00 | 0.00 | 0.00 |
| ECOLI | | | | | | | | | |
| sp\|P0A6B7\|ISCS_ | 2.38 | 0.19 | 1.28 | 0.88 | 0.13 | 0.46 | 11.00 | 0.00 | 0.00 |
| ECOLI | | | | | | | | | |
| sp\|P0AE52\|BCP_ | 2.15 | 0.08 | 1.81 | 0.52 | 0.13 | 0.43 | 8.00 | 0.00 | 0.27 |
| ECOLI | | | | | | | | | |
| sp\|P0AC69\|GLRX4_ | 2.37 | 0.08 | 0.84 | 0.28 | 0.05 | 0.24 | 5.00 | 0.00 | 0.00 |
| ECOLI | | | | | | | | | |
| sp\|P69828\|PTKA_ | 2.38 | 0.20 | 1.08 | 0.81 | 0.08 | 0.19 | 9.00 | 0.00 | 0.00 |
| ECOLI | | | | | | | | | |
| sp\|P0A870\|TALB_ | 1.73 | 0.27 | 1.55 | 0.27 | 0.15 | 0.54 | 10.00 | 0.00 | 0.58 |
| ECOLI | | | | | | | | | |
| sp\|P0AG67\|RS1_ | 1.70 | 0.21 | 1.40 | 0.86 | 0.25 | 0.42 | 16.00 | 0.00 | 0.36 |
| ECOLI | | | | | | | | | |
| sp\|P0A7K2\|RL7_ | 2.25 | 0.10 | 1.34 | 0.97 | 0.12 | 0.39 | 11.00 | 0.00 | 0.01 |
| ECOLI | | | | | | | | | |
| sp\|P0A707\|IF3_ | 2.36 | 0.17 | 1.18 | 0.36 | 0.18 | 0.36 | 6.00 | 0.00 | 0.00 |
| ECOLI | | | | | | | | | |
| sp\|P0AD33\|YFCZ_ | 1.55 | 0.13 | 1.24 | 0.16 | 0.16 | 0.12 | 5.00 | 0.00 | 0.03 |
| ECOLI | | | | | | | | | |
| sp\|P0A867\|TALA_ | 1.47 | 0.10 | 1.06 | 0.21 | 0.09 | 0.26 | 6.00 | 0.00 | 0.01 |
| ECOLI | | | | | | | | | |
| sp\|P13029\|KATG_ | 1.42 | 0.28 | 1.36 | 0.37 | 0.14 | 0.54 | 13.00 | 0.00 | 0.95 |
| ECOLI | | | | | | | | | |
| sp\|P0A6A3\|ACKA_ | 2.58 | 0.09 | 1.03 | 0.85 | 0.10 | 0.39 | 8.00 | 0.00 | 0.00 |
| ECOLI | | | | | | | | | |
| sp\|P0A9P0\|DLDH__ | 1.42 | 0.45 | 1.40 | 0.46 | 0.19 | 0.64 | 21.00 | 0.00 | 0.99 |
| ECOLI | | | | | | | | | |
| sp\|P0A6F5\|CH60_ | 1.23 | 0.48 | 1.15 | 0.38 | 0.22 | 0.52 | 24.00 | 0.00 | 0.82 |
| ECOLI | | | | | | | | | |
| sp\|P0A9K9\|SLYD_ | 1.22 | 0.09 | 1.17 | 0.19 | 0.09 | 0.22 | 6.00 | 0.00 | 0.90 |
| ECOLI | | | | | | | | | |
| sp\|P45578\|LUXS_ | 1.67 | 0.09 | 1.40 | 0.44 | 0.06 | 0.28 | 7.00 | 0.00 | 0.30 |
| ECOLI | | | | | | | | | |
| sp\|P0A6L2\|DAPA_ | 1.68 | 0.39 | 1.16 | 0.29 | 0.19 | 0.26 | 7.00 | 0.00 | 0.01 |
| ECOLI | | | | | | | | | |
| sp\|P0AD61\|KPYK1_ | 1.28 | 0.42 | 1.35 | 0.08 | 0.11 | 0.11 | 5.00 | 0.00 | 0.65 |
| ECOLI | | | | | | | | | |
| sp\|P37188\|PTKB_ | 2.85 | 0.08 | 0.87 | 1.06 | 0.07 | 0.24 | 8.00 | 0.00 | 0.00 |
| ECOLI | | | | | | | | | |
| sp\|P76403\|YEGQ_ | 2.51 | 0.10 | 1.32 | 0.99 | 0.07 | 0.40 | 9.00 | 0.00 | 0.00 |
| ECOLI | | | | | | | | | |
| sp\|P63284\|CLPB_ | 1.98 | 0.16 | 1.50 | 0.77 | 0.24 | 0.56 | 11.00 | 0.00 | 0.17 |
| ECOLI | | | | | | | | | |
| sp\|P0ABU2\|YCHF_ | 1.74 | 0.30 | 1.17 | 0.49 | 0.32 | 0.32 | 9.00 | 0.00 | 0.02 |
| ECOLI | | | | | | | | | |
| sp\|P0A7J7\|RL11_ | 2.44 | 0.10 | 0.93 | 0.88 | 0.13 | 0.38 | 8.00 | 0.00 | 0.00 |
| ECOLI | | | | | | | | | |
| sp\|P0A7K6\|RL19_ | 2.42 | 0.21 | 0.96 | 0.29 | 0.07 | 0.12 | 4.00 | 0.00 | 0.00 |
| ECOLI | | | | | | | | | |
| sp\|P0A7J3\|RL10_ | 1.73 | 0.09 | 1.07 | 0.21 | 0.08 | 0.30 | 5.00 | 0.00 | 0.00 |
| ECOLI | | | | | | | | | |
| sp\|P0A6K3\|DEF_ | 1.87 | 0.09 | 1.50 | 0.50 | 0.08 | 0.18 | 6.00 | 0.00 | 0.19 |
| ECOLI | | | | | | | | | |
| sp\|P0AF28\|NARL_ | 1.64 | 0.37 | 1.23 | 0.15 | 0.13 | 0.33 | 6.00 | 0.00 | 0.03 |
| ECOLI | | | | | | | | | |
| sp\|P0A746\|MSRB_ | 1.74 | 0.11 | 1.09 | 0.12 | 0.08 | 0.20 | 4.00 | 0.00 | 0.00 |
| ECOLI | | | | | | | | | |
| sp\|P0AES4\|GYRA_ | 2.16 | 0.12 | 1.45 | 0.69 | 0.13 | 0.30 | 7.00 | 0.00 | 0.03 |
| ECOLI | | | | | | | | | |
| sp\|P68066\|GRCA_ | 1.88 | 0.24 | 1.01 | 0.64 | 0.20 | 0.27 | 8.00 | 0.00 | 0.00 |
| ECOLI | | | | | | | | | |
| sp\|P77433\|YKGG_ | 2.39 | 0.15 | 0.89 | 0.61 | 0.22 | 0.33 | 6.00 | 0.00 | 0.00 |
| ECOLI | | | | | | | | | |
| sp\|P60438\|RL3_ | 2.54 | 0.17 | 0.97 | 0.39 | 0.04 | 0.10 | 4.00 | 0.00 | 0.00 |
| ECOLI | | | | | | | | | |
| sp\|P0AF93\|RIDA_ | 1.88 | 0.27 | 1.24 | 0.22 | 0.03 | 0.18 | 4.00 | 0.00 | 0.00 |
| ECOLI | | | | | | | | | |
| sp\|P06610\|BTUE_ | 2.62 | 0.10 | 1.48 | 1.49 | 0.16 | 0.93 | 13.00 | 0.00 | 0.03 |
| ECOLI | | | | | | | | | |
| sp\|P25539\|RIBD_ | 1.95 | 0.12 | 1.34 | 0.45 | 0.17 | 0.16 | 5.00 | 0.00 | 0.03 |
| ECOLI | | | | | | | | | |
| sp\|P0AG51\|RL30_ | 2.41 | 0.20 | 0.76 | 0.40 | 0.06 | 0.13 | 4.00 | 0.00 | 0.00 |
| ECOLI | | | | | | | | | |
| sp\|P68679\|RS21_ | 2.52 | 0.22 | 0.79 | 0.16 | 0.12 | 0.09 | 3.00 | 0.00 | 0.00 |
| ECOLI | | | | | | | | | |
| sp\|P0A7M9\|RL31_ | 2.18 | 0.19 | 0.90 | 0.52 | 0.15 | 0.17 | 5.00 | 0.00 | 0.00 |
| ECOLI | | | | | | | | | |
| sp\|P0A6P1\|EFTS_ | 1.58 | 0.25 | 1.36 | 0.30 | 0.09 | 0.21 | 5.00 | 0.00 | 0.34 |
| ECOLI | | | | | | | | | |
| sp\|P75691\|YAHK_ | 1.34 | 0.49 | 1.28 | 0.21 | 0.16 | 0.26 | 7.00 | 0.00 | 0.89 |
| ECOLI | | | | | | | | | |
| sp\|P0A9M8\|PTA_ | 1.91 | 0.12 | 1.32 | 0.92 | 0.13 | 0.32 | 9.00 | 0.00 | 0.14 |
| ECOLI | | | | | | | | | |
| sp\|P0A6P9\|ENO_ | 1.89 | 0.21 | 1.33 | 0.79 | 0.11 | 0.27 | 8.00 | 0.00 | 0.11 |
| ECOLI | | | | | | | | | |
| sp\|P0AE08\|AHPC_ | 1.74 | 0.18 | 1.31 | 0.70 | 0.10 | 0.34 | 8.00 | 0.00 | 0.22 |
| ECOLI | | | | | | | | | |
| sp\|P0AEE5\|DGAL_ | 1.48 | 0.59 | 1.20 | 0.12 | 0.04 | 0.15 | 4.00 | 0.00 | 0.03 |
| ECOLI | | | | | | | | | |
| sp\|P0A7Z4\|RPOA_ | 2.74 | 0.09 | 1.54 | 1.50 | 0.07 | 0.71 | 10.00 | 0.00 | 0.04 |
| ECOLI | | | | | | | | | |
| sp\|P0AGJ5\|YFIF_ | 2.51 | 0.04 | 1.26 | 0.45 | 0.07 | 0.30 | 4.00 | 0.00 | 0.00 |
| ECOLI | | | | | | | | | |
| sp\|P63020\|NFUA_ | 1.67 | 0.07 | 1.31 | 0.23 | 0.06 | 0.27 | 4.00 | 0.00 | 0.13 |
| ECOLI | | | | | | | | | |
| sp\|P18196\|MINC_ | 2.18 | 0.08 | 1.15 | 0.19 | 0.04 | 0.16 | 3.00 | 0.00 | 0.00 |
| ECOLI | | | | | | | | | |
| sp\|P02358\|RS6_ | 1.98 | 0.12 | 0.80 | 0.24 | 0.08 | 0.33 | 4.00 | 0.00 | 0.00 |
| ECOLI | | | | | | | | | |
| sp\|P0AGE9\|SUCD_ | 1.84 | 0.14 | 1.40 | 0.30 | 0.04 | 0.24 | 4.00 | 0.00 | 0.08 |
| ECOLI | | | | | | | | | |
| sp\|P76177\|YDGH_ | 1.67 | 0.27 | 1.29 | 0.70 | 0.14 | 0.70 | 11.00 | 0.00 | 0.34 |
| ECOLI | | | | | | | | | |
| sp\|P0AGE0\|SSB_ | 2.36 | 0.28 | 1.46 | 0.93 | 0.16 | 0.70 | 8.00 | 0.00 | 0.06 |
| ECOLI | | | | | | | | | |
| sp\|P23836\|PHOP_ | 1.75 | 0.22 | 1.25 | 0.33 | 0.15 | 0.19 | 4.00 | 0.00 | 0.06 |
| ECOLI | | | | | | | | | |
| sp\|P68919\|RL25_ | 2.27 | 0.11 | 0.82 | 0.76 | 0.11 | 0.21 | 5.00 | 0.00 | 0.00 |
| ECOLI | | | | | | | | | |
| sp\|P60560\|GUAC_ | 1.58 | 0.11 | 1.03 | 0.49 | 0.06 | 0.28 | 5.00 | 0.00 | 0.08 |
| ECOLI | | | | | | | | | |
| sp\|P23843\|OPPA_ | 1.68 | 0.26 | 1.93 | 0.54 | 0.07 | 0.40 | 6.00 | 0.00 | 0.57 |
| ECOLI | | | | | | | | | |
| sp\|P0A7R1\|RL9_ | 1.76 | 0.21 | 1.16 | 0.42 | 0.14 | 0.41 | 5.00 | 0.00 | 0.07 |
| ECOLI | | | | | | | | | |
| sp\|P0AFG8\|ODP1_ | 0.68 | 0.14 | 1.99 | 0.24 | 0.10 | 0.65 | 24.00 | 0.00 | 0.00 |
| ECOLI | | | | | | | | | |
| sp\|P0A805\|RRF_ | 1.67 | 0.21 | 1.31 | 0.33 | 0.09 | 0.28 | 4.00 | 0.00 | 0.22 |
| ECOLI | | | | | | | | | |
| sp\|P61889\|MDH_ | 1.37 | 0.18 | 1.65 | 0.43 | 0.14 | 0.61 | 8.00 | 0.00 | 0.50 |
| ECOLI | | | | | | | | | |
| sp\|P0ACF4\|DBHB_ | 1.93 | 0.17 | 1.19 | 0.19 | 0.03 | 0.25 | 3.00 | 0.00 | 0.01 |
| ECOLI | | | | | | | | | |
| sp\|P76440\|PRET_ | 1.12 | 0.11 | 1.55 | 0.18 | 0.09 | 0.24 | 4.00 | 0.00 | 0.04 |
| ECOLI | | | | | | | | | |
| sp\|P0AC38\|ASPA_ | 1.99 | 0.17 | 1.47 | 0.78 | 0.13 | 0.52 | 6.00 | 0.00 | 0.30 |
| ECOLI | | | | | | | | | |
| sp\|P69441\|KAD_ | 1.81 | 0.33 | 1.31 | 0.55 | 0.26 | 0.47 | 6.00 | 0.00 | 0.22 |
| ECOLI | | | | | | | | | |
| sp\|P04805\|SYE_ | 2.06 | 0.08 | 1.76 | 0.80 | 0.08 | 0.66 | 6.00 | 0.00 | 0.74 |
| ECOLI | | | | | | | | | |
| sp\|P0A9Q7\|ADHE_ | 1.92 | 0.17 | 1.81 | 1.24 | 0.18 | 0.83 | 11.00 | 0.00 | 0.97 |
| ECOLI | | | | | | | | | |
| sp\|P0AD59\|IVY_ | 1.45 | 0.55 | 0.91 | 0.34 | 0.15 | 0.33 | 6.00 | 0.00 | 0.03 |
| ECOLI | | | | | | | | | |
| sp\|P0A8M3\|SYT_ | 1.65 | 0.01 | 0.86 | 0.36 | 0.02 | 0.05 | 3.00 | 0.00 | 0.02 |
| ECOLI | | | | | | | | | |
| sp\|P69797\|PTNAB_ | 1.60 | 0.30 | 1.29 | 0.36 | 0.08 | 0.31 | 4.00 | 0.00 | 0.36 |
| ECOLI | | | | | | | | | |
| sp\|P0A6K6\|DEOB_ | 2.05 | 0.24 | 1.14 | 0.91 | 0.23 | 0.71 | 7.00 | 0.00 | 0.08 |
| ECOLI | | | | | | | | | |
| sp\|P0AA16\|OMPR_ | 2.13 | 0.36 | 1.07 | 0.90 | 0.69 | 0.60 | 8.00 | 0.00 | 0.04 |
| ECOLI | | | | | | | | | |
| sp\|P0A6F1\|CARA_ | 1.61 | 0.09 | 1.37 | 0.15 | 0.09 | 0.32 | 3.00 | 0.00 | 0.48 |
| ECOLI | | | | | | | | | |
| sp\|P15639\|PUR9_ | 1.54 | 0.09 | 1.02 | 0.38 | 0.06 | 0.39 | 4.00 | 0.00 | 0.15 |
| ECOLI | | | | | | | | | |
| sp\|P0A7L0\|RL1_ | 2.54 | 0.29 | 0.93 | 0.50 | 0.10 | 0.20 | 3.00 | 0.00 | 0.01 |
| ECOLI | | | | | | | | | |
| sp\|P0A799\|PGK_ | 1.34 | 0.15 | 1.29 | 0.18 | 0.14 | 0.18 | 3.00 | 0.00 | 0.96 |
| ECOLI | | | | | | | | | |
| sp\|P0A825\|GLYA_ | 1.58 | 0.28 | 1.16 | 0.56 | 0.25 | 0.60 | 7.00 | 0.00 | 0.34 |
| ECOLI | | | | | | | | | |
| sp\|P05042\|FUMC_ | 1.62 | 0.14 | 2.03 | 1.04 | 0.29 | 1.06 | 13.00 | 0.00 | 0.53 |
| ECOLI | | | | | | | | | |
| sp\|P0A9N4\|PFLA_ | 2.23 | 0.15 | 1.11 | 0.13 | 0.08 | 0.02 | 2.00 | 0.00 | 0.01 |
| ECOLI | | | | | | | | | |
| sp\|P0A763\|NDK_ | 1.55 | 0.23 | 1.28 | 0.83 | 0.23 | 0.64 | 9.00 | 0.00 | 0.68 |
| ECOLI | | | | | | | | | |
| sp\|P00350\|6PGD_ | 1.24 | 0.13 | 1.58 | 0.14 | 0.09 | 0.41 | 4.00 | 0.00 | 0.26 |
| ECOLI | | | | | | | | | |
| sp\|P25526\|GABD_ | 1.57 | 0.10 | 1.29 | 0.26 | 0.03 | 0.28 | 3.00 | 0.00 | 0.41 |
| ECOLI | | | | | | | | | |
| sp\|P23847\|DPPA_ | 0.92 | 0.25 | 1.35 | 0.09 | 0.10 | 0.12 | 3.00 | 0.00 | 0.01 |
| ECOLI | | | | | | | | | |
| sp\|P0A6Y5\|HSLO_ | 1.36 | 0.51 | 0.95 | 0.25 | 0.19 | 0.31 | 5.00 | 0.00 | 0.10 |
| ECOLI | | | | | | | | | |
| sp\|P0ACF0\|DBHA_ | 2.02 | 0.16 | 1.19 | 0.48 | 0.03 | 0.16 | 3.00 | 0.00 | 0.05 |
| ECOLI | | | | | | | | | |
| sp\|P69910\|DCEB_ | 1.43 | 0.61 | 1.29 | 0.03 | 0.10 | 0.20 | 3.00 | 0.00 | 0.47 |
| ECOLI; | | | | | | | | | |
| sp\|P69908\|DCEA_ | | | | | | | | | |
| ECOLI | | | | | | | | | |
| sp\|P0A8E1\|YCFP_ | 1.75 | 0.17 | 0.97 | 0.35 | 0.11 | 0.25 | 3.00 | 0.00 | 0.04 |
| ECOLI | | | | | | | | | |
| sp\|P0ABE2\|BOLA_ | 1.88 | 0.13 | 0.81 | 0.00 | 0.02 | 0.16 | 2.00 | 0.00 | 0.01 |
| ECOLI | | | | | | | | | |
| sp\|P0A8E7\|YAJQ_ | 1.90 | 0.36 | 1.60 | 0.57 | 0.12 | 0.29 | 4.00 | 0.00 | 0.59 |
| ECOLI | | | | | | | | | |
| sp\|P0AEZ9\|MOAB_ | 1.17 | 0.30 | 1.80 | 0.24 | 0.25 | 0.60 | 8.00 | 0.00 | 0.03 |
| ECOLI | | | | | | | | | |
| sp\|P0AG48\|RL21_ | 2.59 | 0.20 | 0.92 | 0.18 | 0.01 | 0.14 | 2.00 | 0.00 | 0.01 |
| ECOLI | | | | | | | | | |
| sp\|P0A7E5\|PYRG_ | 2.65 | 0.16 | 1.27 | 0.89 | 0.23 | 0.54 | 4.00 | 0.00 | 0.05 |
| ECOLI | | | | | | | | | |
| sp\|P0AET2\|HDEB_ | 0.93 | 0.16 | 1.53 | 0.19 | 0.10 | 0.25 | 4.00 | 0.00 | 0.01 |
| ECOLI | | | | | | | | | |
| sp\|P0A7S9\|RS13_ | 1.72 | 0.07 | 1.05 | 0.06 | 0.09 | 0.11 | 2.00 | 0.00 | 0.02 |
| ECOLI | | | | | | | | | |
| sp\|P00448\|SODM_ | 1.32 | 0.28 | 1.29 | 0.24 | 0.10 | 0.15 | 3.00 | 0.00 | 0.99 |
| ECOLI | | | | | | | | | |
| sp\|P07013\|PRIB_ | 1.99 | 0.13 | 1.14 | 0.15 | 0.10 | 0.03 | 2.00 | 0.00 | 0.02 |
| ECOLI | | | | | | | | | |
| sp\|P69783\|PTGA_ | 1.57 | 0.53 | 1.40 | 0.49 | 0.17 | 0.46 | 6.00 | 0.00 | 0.79 |
| ECOLI | | | | | | | | | |
| sp\|P27550\|ACSA_ | 1.22 | 0.14 | 1.14 | 0.10 | 0.06 | 0.32 | 3.00 | 0.00 | 0.92 |
| ECOLI | | | | | | | | | |
| sp\|P37773\|MPL_ | 1.53 | 0.20 | 1.00 | 0.11 | 0.11 | 0.03 | 2.00 | 0.00 | 0.04 |
| ECOLI | | | | | | | | | |
| sp\|P07004\|PROA_ | 3.21 | 0.22 | 2.61 | 0.37 | 0.01 | 0.06 | 2.00 | 0.00 | 0.18 |
| ECOLI | | | | | | | | | |
| sp\|P16456\|SELD_ | 2.64 | 0.28 | 1.22 | 0.26 | 0.13 | 0.08 | 2.00 | 0.00 | 0.02 |
| ECOLI | | | | | | | | | |
| sp\|P07395\|SYFB_ | 1.47 | 0.33 | 1.28 | 0.62 | 0.20 | 0.63 | 7.00 | 0.00 | 0.83 |
| ECOLI | | | | | | | | | |
| sp\|P0A9D4\|CYSE_ | 1.81 | 0.20 | 0.81 | 0.53 | 0.13 | 0.58 | 4.00 | 0.01 | 0.05 |
| ECOLI | | | | | | | | | |
| sp\|P0AEU7\|SKP_ | 1.19 | 0.33 | 1.40 | 0.16 | 0.11 | 0.38 | 4.00 | 0.01 | 0.51 |
| ECOLI | | | | | | | | | |
| sp\|P0ABP8\|DEOD_ | 1.16 | 0.31 | 1.69 | 0.22 | 0.11 | 0.17 | 3.00 | 0.01 | 0.04 |
| ECOLI | | | | | | | | | |
| sp\|P0A953\|FABB_ | 2.15 | 0.12 | 1.59 | 1.08 | 0.11 | 0.60 | 5.00 | 0.01 | 0.52 |
| ECOLI | | | | | | | | | |
| sp\|P39831\|YDFG_ | 2.23 | 0.14 | 1.46 | 0.58 | 0.01 | 0.43 | 3.00 | 0.01 | 0.19 |
| ECOLI | | | | | | | | | |
| sp\|P76558\|MAO2_ | 0.96 | 0.37 | 1.19 | 0.30 | 0.25 | 0.41 | 9.00 | 0.01 | 0.36 |
| ECOLI | | | | | | | | | |
| sp\|P0A780\|NUSB_ | 2.07 | 0.13 | 1.09 | 0.08 | 0.11 | 0.24 | 2.00 | 0.01 | 0.03 |
| ECOLI | | | | | | | | | |
| sp\|P31142\|THTM_ | 3.60 | 0.12 | 1.76 | 1.66 | 0.08 | 0.63 | 4.00 | 0.01 | 0.11 |
| ECOLI | | | | | | | | | |
| sp\|P76569\|YFGD_ | 2.57 | 0.03 | 1.78 | 0.38 | 0.04 | 0.06 | 2.00 | 0.01 | 0.11 |
| ECOLI | | | | | | | | | |
| sp\|P0ABB0\|ATPA_ | 1.26 | 0.28 | 0.94 | 0.37 | 0.36 | 0.37 | 5.00 | 0.01 | 0.44 |
| ECOLI | | | | | | | | | |
| sp\|P0AEN1\|FRE_ | 2.19 | 0.02 | 0.95 | 0.60 | 0.04 | 0.53 | 3.00 | 0.01 | 0.06 |
| ECOLI | | | | | | | | | |
| sp\|P65556\|YFCD_ | 1.71 | 0.09 | 1.11 | 0.54 | 0.06 | 0.66 | 4.00 | 0.01 | 0.30 |
| ECOLI | | | | | | | | | |
| sp\|P39274\|YJDJ_ | 2.78 | 0.09 | 0.86 | 0.99 | 0.12 | 0.13 | 3.00 | 0.01 | 0.03 |
| ECOLI | | | | | | | | | |
| sp\|P0ACI0\|ROB_ | 1.29 | 0.07 | 0.66 | 0.15 | 0.10 | 0.06 | 2.00 | 0.01 | 0.04 |
| ECOLI | | | | | | | | | |
| sp\|P37903\|USPF_ | 2.16 | 0.13 | 1.15 | 0.19 | 0.06 | 0.25 | 2.00 | 0.01 | 0.04 |
| ECOLI | | | | | | | | | |
| sp\|P60906\|SYH_ | 1.39 | 0.09 | 1.00 | 0.18 | 0.09 | 0.06 | 2.00 | 0.01 | 0.13 |
| ECOLI | | | | | | | | | |
| sp\|P15034\|AMPP_ | 1.39 | 0.90 | 1.12 | 0.27 | 0.18 | 0.19 | 6.00 | 0.01 | 0.16 |
| ECOLI | | | | | | | | | |
| sp\|P0AE18\|MAP1_ | 1.42 | 0.13 | 0.74 | 0.41 | 0.11 | 0.26 | 3.00 | 0.01 | 0.09 |
| ECOLI | | | | | | | | | |
| sp\|P18843\|NADE_ | 2.06 | 0.26 | 0.79 | 0.63 | 0.25 | 0.12 | 3.00 | 0.01 | 0.03 |
| ECOLI | | | | | | | | | |
| sp\|P15288\|PEPD_ | 1.69 | 0.23 | 1.33 | 0.06 | 0.13 | 0.19 | 2.00 | 0.01 | 0.20 |
| ECOLI | | | | | | | | | |
| sp\|P0A6W9\|GSH1_ | 1.99 | 0.31 | 1.89 | 0.58 | 0.14 | 0.73 | 4.00 | 0.01 | 0.99 |
| ECOLI | | | | | | | | | |
| sp\|P0AFK0\|PMBA_ | 1.50 | 0.54 | 1.27 | 0.35 | 0.21 | 0.36 | 4.00 | 0.01 | 0.65 |
| ECOLI | | | | | | | | | |
| sp\|P0ACC7\|GLMU_ | 1.82 | 0.07 | 0.94 | 0.59 | 0.11 | 0.41 | 3.00 | 0.01 | 0.14 |
| ECOLI | | | | | | | | | |
| sp\|P62768\|YAEH_ | 2.76 | 0.01 | 1.49 | 1.39 | 0.03 | 0.74 | 4.00 | 0.01 | 0.23 |
| ECOLI | | | | | | | | | |
| sp\|P0AF96\|TABA_ | 1.75 | 0.06 | 1.87 | 0.10 | 0.08 | 0.29 | 2.00 | 0.01 | 0.87 |
| ECOLI | | | | | | | | | |
| sp\|P0A7V0\|RS2_ | 2.72 | 0.11 | 0.83 | 1.05 | 0.09 | 0.28 | 3.00 | 0.01 | 0.04 |
| ECOLI | | | | | | | | | |
| sp\|P0AFR4\|YCIO_ | 1.68 | 0.26 | 0.69 | 0.30 | 0.46 | 0.25 | 3.00 | 0.01 | 0.05 |
| ECOLI | | | | | | | | | |
| sp\|P0C0L2\|OSMC_ | 1.87 | 0.10 | 1.29 | 0.11 | 0.14 | 0.30 | 2.00 | 0.01 | 0.17 |
| ECOLI | | | | | | | | | |
| sp\|P0A705\|IF2_ | 1.86 | 0.18 | 1.75 | 0.89 | 0.20 | 1.96 | 12.00 | 0.01 | 0.99 |
| ECOLI | | | | | | | | | |
| sp\|P0AFF6\|NUSA_ | 1.80 | 0.07 | 0.99 | 0.08 | 0.09 | 0.33 | 2.00 | 0.01 | 0.09 |
| ECOLI | | | | | | | | | |
| sp\|P22188\|MURE_ | 2.22 | 0.09 | 1.21 | 0.04 | 0.13 | 0.42 | 2.00 | 0.02 | 0.09 |
| ECOLI | | | | | | | | | |
| sp\|P60651\|SPEB_ | 1.29 | 0.30 | 1.44 | 0.06 | 0.04 | 0.20 | 2.00 | 0.02 | 0.57 |
| ECOLI | | | | | | | | | |
| sp\|P0AEK2\|FABG_ | 1.69 | 0.30 | 1.09 | 0.12 | 0.24 | 0.11 | 2.00 | 0.02 | 0.11 |
| ECOLI | | | | | | | | | |
| sp\|P05459\|PDXB_ | 2.46 | 0.29 | 1.77 | 0.87 | 0.10 | 1.02 | 4.00 | 0.02 | 0.51 |
| ECOLI | | | | | | | | | |
| sp\|P0A7G2\|RBFA_ | 2.08 | 0.19 | 1.68 | 0.19 | 0.27 | 0.25 | 2.00 | 0.02 | 0.40 |
| ECOLI | | | | | | | | | |
| sp\|P09158\|SPEE_ | 2.53 | 0.30 | 1.11 | 0.95 | 0.35 | 0.13 | 3.00 | 0.02 | 0.09 |
| ECOLI | | | | | | | | | |
| sp\|P25553\|ALDA_ | 1.07 | 0.44 | 1.71 | 0.18 | 0.04 | 0.22 | 3.00 | 0.02 | 0.02 |
| ECOLI | | | | | | | | | |
| sp\|P0AAC8\|ISCA_ | 1.88 | 0.32 | 0.93 | 0.57 | 0.31 | 0.31 | 3.00 | 0.02 | 0.10 |
| ECOLI | | | | | | | | | |
| sp\|P28904\|TREC_ | 1.90 | 0.35 | 1.09 | 0.58 | 0.36 | 0.25 | 3.00 | 0.02 | 0.17 |
| ECOLI | | | | | | | | | |
| sp\|Q57261\|TRUD_ | 3.28 | 0.05 | 1.24 | 1.52 | 0.05 | 0.16 | 3.00 | 0.02 | 0.10 |
| ECOLI | | | | | | | | | |
| sp\|P0ABS1\|DKSA_ | 1.98 | 0.11 | 1.42 | 0.81 | 0.13 | 0.88 | 4.00 | 0.02 | 0.58 |
| ECOLI | | | | | | | | | |
| sp\|P07813\|SYL_ | 2.94 | 0.50 | 1.63 | 1.09 | 0.18 | 0.37 | 3.00 | 0.02 | 0.17 |
| ECOLI | | | | | | | | | |
| sp\|P00961\|SYGB_ | 2.46 | 0.21 | 1.18 | 0.55 | 0.03 | 0.10 | 2.00 | 0.02 | 0.09 |
| ECOLI | | | | | | | | | |
| sp\|P0A6F9\|CH10_ | 1.72 | 0.56 | 1.73 | 0.34 | 0.10 | 0.68 | 4.00 | 0.02 | 1.00 |
| ECOLI | | | | | | | | | |
| sp\|P0AEZ3\|MIND_ | 1.99 | 0.20 | 0.85 | 0.16 | 0.16 | 0.38 | 2.00 | 0.02 | 0.07 |
| ECOLI | | | | | | | | | |
| sp\|P0A7M2\|RL28_ | 2.25 | 0.20 | 0.85 | 0.50 | 0.14 | 0.04 | 2.00 | 0.02 | 0.06 |
| ECOLI | | | | | | | | | |
| sp\|P0A9S3\|GATD_ | 1.70 | 0.35 | 1.05 | 0.37 | 0.28 | 0.50 | 3.00 | 0.03 | 0.24 |
| ECOLI | | | | | | | | | |
| sp\|P45470\|YHBO_ | 1.53 | 0.21 | 1.30 | 0.61 | 0.16 | 0.21 | 3.00 | 0.03 | 0.81 |
| ECOLI | | | | | | | | | |
| sp\|P0A8L1\|SYS_ | 1.15 | 0.11 | 1.29 | 0.26 | 0.08 | 0.04 | 2.00 | 0.03 | 0.76 |
| ECOLI | | | | | | | | | |
| sp\|P0ACY1\|YDJA_ | 2.43 | 0.00 | 1.06 | 0.56 | 0.00 | 0.33 | 2.00 | 0.03 | 0.11 |
| ECOLI | | | | | | | | | |
| sp\|P0A9M2\|HPRT_ | 1.94 | 0.28 | 1.08 | 0.15 | 0.39 | 0.21 | 2.00 | 0.03 | 0.14 |
| ECOLI | | | | | | | | | |
| sp\|P63177\|RLMB_ | 2.52 | 0.26 | 1.39 | 0.54 | 0.05 | 0.38 | 2.00 | 0.04 | 0.16 |
| ECOLI | | | | | | | | | |
| sp\|P62707\|GPMA_ | 0.71 | 0.21 | 1.04 | 0.08 | 0.07 | 0.10 | 2.00 | 0.04 | 0.09 |
| ECOLI | | | | | | | | | |
| sp\|P0A7W1\|RS5_ | 2.25 | 0.24 | 1.38 | 0.36 | 0.11 | 0.49 | 2.00 | 0.04 | 0.23 |
| ECOLI | | | | | | | | | |
| sp\|P42641\|OBG_ | 1.79 | 0.04 | 1.12 | 0.02 | 0.05 | 0.54 | 2.00 | 0.04 | 0.28 |
| ECOLI | | | | | | | | | |
| sp\|P0A6J8\|DDLA_ | 2.67 | 0.02 | 1.66 | 0.82 | 0.03 | 0.18 | 2.00 | 0.04 | 0.29 |
| ECOLI | | | | | | | | | |
| sp\|P0A6H5\|HSLU_ | 2.20 | 0.22 | 0.92 | 0.48 | 0.24 | 0.34 | 2.00 | 0.04 | 0.12 |
| ECOLI | | | | | | | | | |
| sp\|P0A800\|RPOZ_ | 1.90 | 0.04 | 2.08 | 0.18 | 0.06 | 0.57 | 2.00 | 0.04 | 0.91 |
| ECOLI | | | | | | | | | |
| sp\|P0A6V8\|GLK_ | 2.46 | 0.15 | 1.15 | 0.60 | 0.22 | 0.40 | 2.00 | 0.04 | 0.15 |
| ECOLI | | | | | | | | | |
| sp\|P0ABH7\|CISY_ | 1.11 | 0.14 | 1.76 | 0.67 | 0.17 | 0.98 | 8.00 | 0.05 | 0.22 |
| ECOLI | | | | | | | | | |
| sp\|P00363\|FRDA_ | 1.52 | 0.03 | 0.76 | 0.50 | 0.04 | 0.01 | 2.00 | 0.05 | 0.20 |
| ECOLI | | | | | | | | | |
| sp\|P0A6N4\|EFP_ | 1.39 | 0.10 | 0.69 | 0.33 | 0.14 | 0.24 | 2.00 | 0.05 | 0.18 |
| ECOLI | | | | | | | | | |
| sp\|P0AFG0\|NUSG_ | 1.48 | 0.09 | 0.91 | 0.95 | 0.15 | 0.50 | 4.00 | 0.05 | 0.49 |
| ECOLI | | | | | | | | | |
| sp\|P777187\|THII_ | 1.69 | 0.05 | 0.96 | 0.51 | 0.07 | 0.20 | 2.00 | 0.05 | 0.25 |
| ECOLI | | | | | | | | | |
| sp\|P0A8V2\|RPOB_ | 1.53 | 0.47 | 1.45 | 0.54 | 0.37 | 0.51 | 4.00 | 0.05 | 0.99 |
| ECOLI | | | | | | | | | |

E. coli Tryptophanase is Inhibited by S-Sulfhydration

Next, the studies focused on connecting the S-sulfhydrome analysis to the phenotypes observed in the CKD preclinical model. One of the top 10 most abundant E. coli S-sulfhydrated proteins was tryptophanase (TnaA) (FIG. 6F). TnaA is a secreted enzyme that catalyzes the degradation of tryptophan to indole, pyruvate, and ammonia. Indoles are a class of bacterial-produced molecules that not only regulate bacterial physiology (Darkoh et al., 2019; Lee et al., 2008), but also participate in bacteria-host interactions (Kumar and Sperandio, 2019; Wlodarska et al., 2017; Zelante et al, 2013). Indoles can be transported through the portal vein to the liver where they are oxidized, yielding the uremic toxin indoxyl sulfate (Leong and Sirich, 2016).

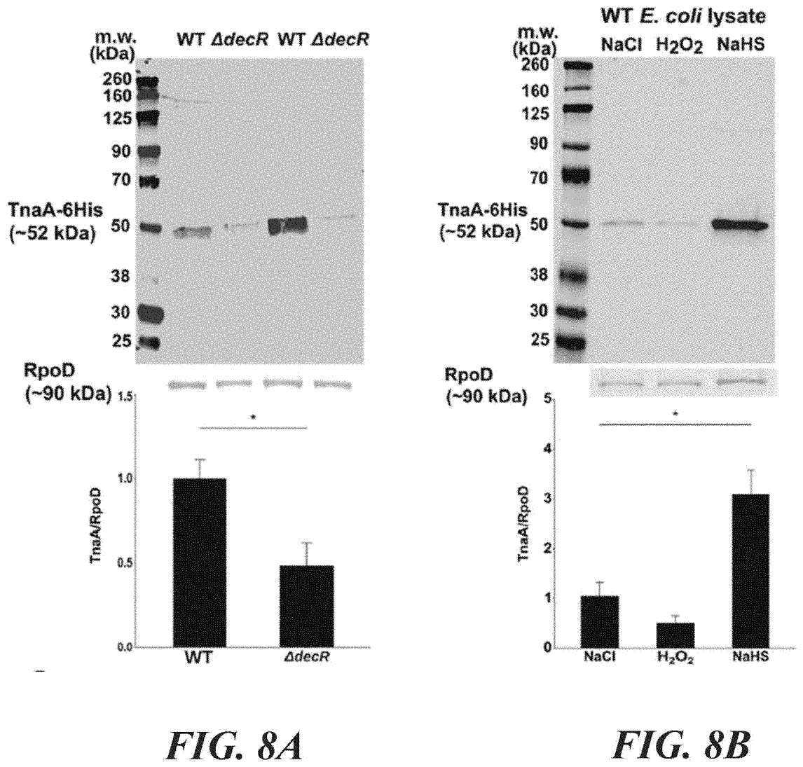

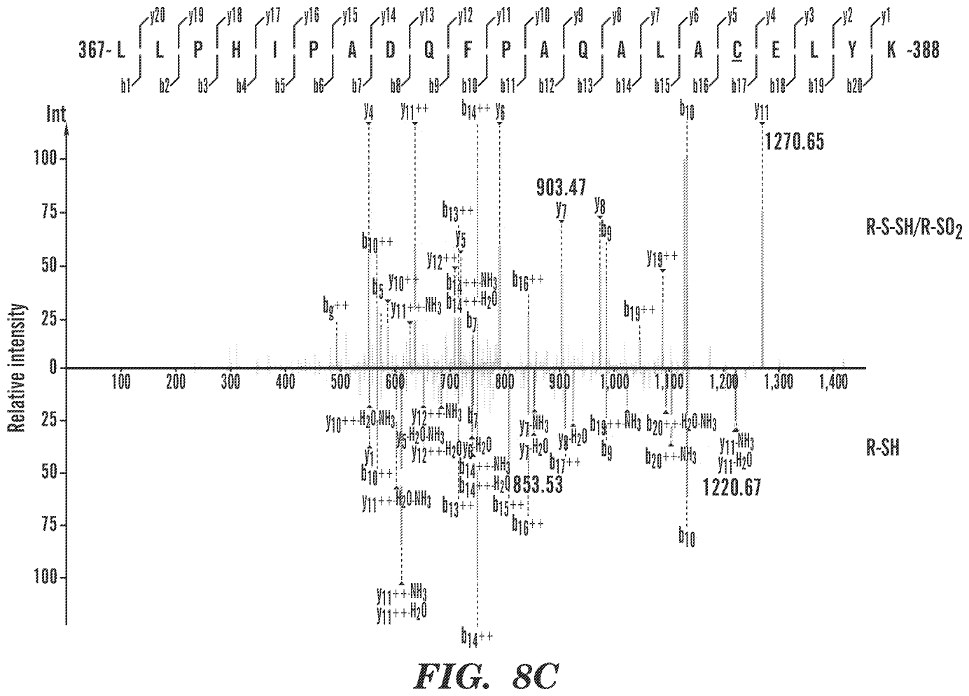

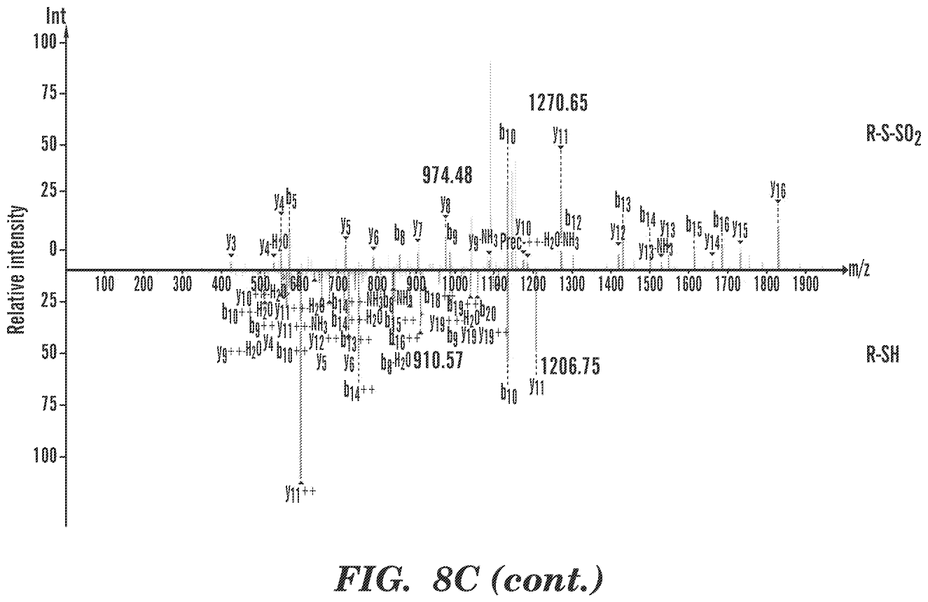

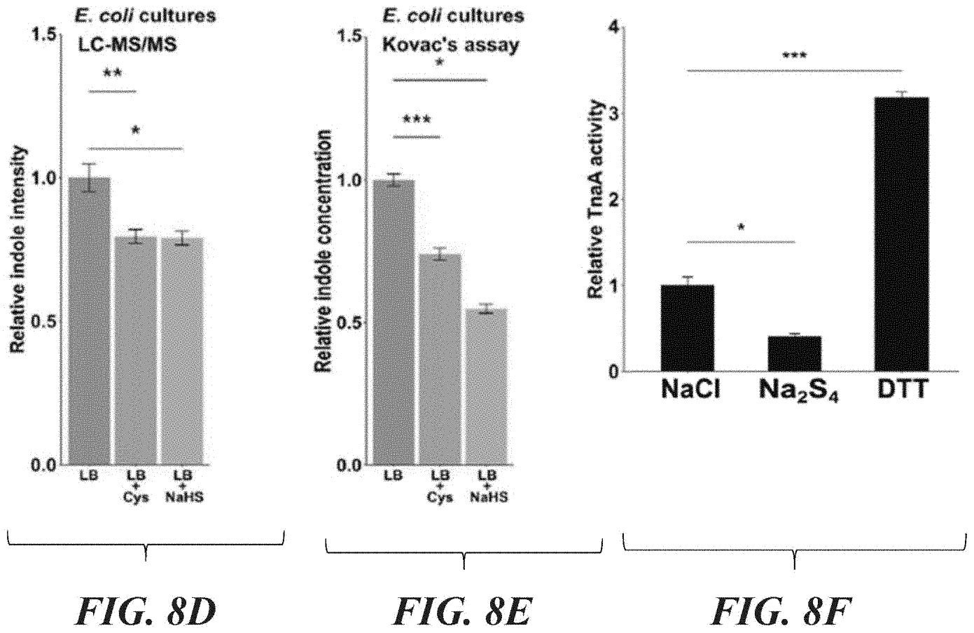

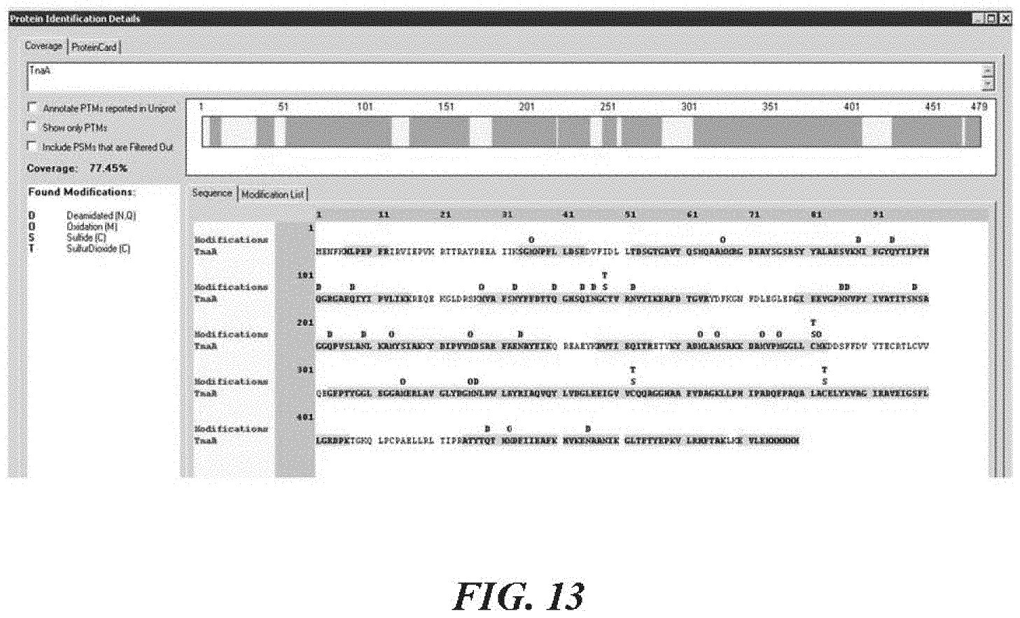

For these reasons, TnaA emerged as an attractive target for investigating host-microbe interactions in the CKD mouse model. The E. coli TnaA chromosomal copy was replaced with a cloned tnaA-his under its native promoter. It was then validated that the S-sulfhydrome results by analyzing TnaA S-sulfhydration in WT vs ΔdecR E. coli lysates using Western blot analysis and found reduced TnaA S-sulfhydration in ΔdecR lysates (FIG. 8A). E. coli lysates treated with H₂O₂ and NaHS showed reduced and increased TnaA S-sulfhydration, respectively (FIG. 8B). Since the S-sulfhydration pull-down method reduces the S-sulfhydrated cysteine residue (i.e. removes the S-sulfhydration), it could not pinpoint the exact cysteine residues being S-sulfhydrated, as TnaA has 7 cysteines. Therefore, the natively expressed TnaA-His was purified from E. coli grown in LB supplemented with cysteine and performed LC-MS/MS analysis to detect and map the S-sulfhydration. Several TnaA-His peptides were detected that had a +32 Da addition, matching the molecular weight of S-sulfhydration on a cysteine residue (FIG. 8C). As oxidation of a cysteine residue to sulfinic acid (R—S—O2) results in same mass shift and given the potential for oxidation during the analysis, it could not rule out that such oxidation occurs. However, an S-sulfhydrated cysteine can be oxidized to sulfinic acid (R—S—S—O2) resulting in a +64 Da increase, a shift that results from oxidation of an S-sulfhydrated cysteine or a second S-sulfhydration (R—S—S—SH). These experiments were able to detect a +64 Da shift in several cysteine residues of TnaA (FIG. 8C and FIG. 13). While the studies show evidence that 6 out of the 7 TnaA cysteine residues were S-sulfhydrated (C148, C281, C294, C298, C352 and C383), it could not rule out that cysteine residue (C413) can also be S-sulfhydrated, as the coverage of TnaA (˜78%) did not include peptides with high confidence within this region.

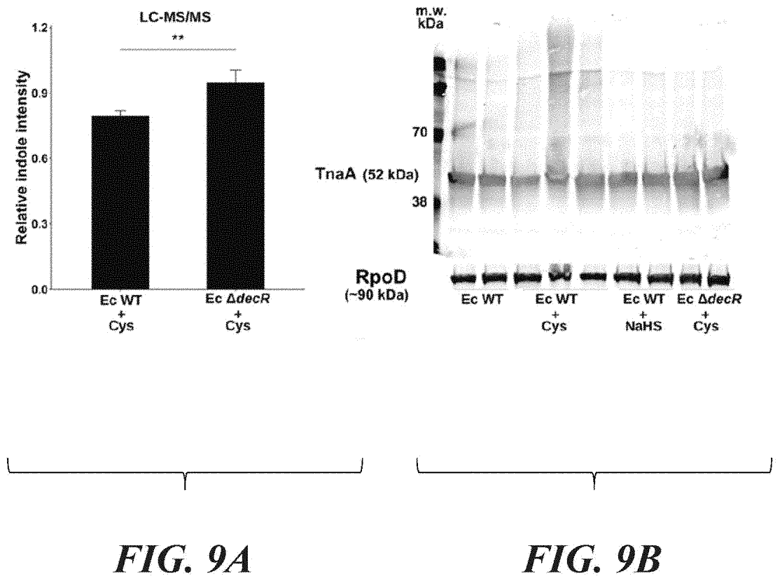

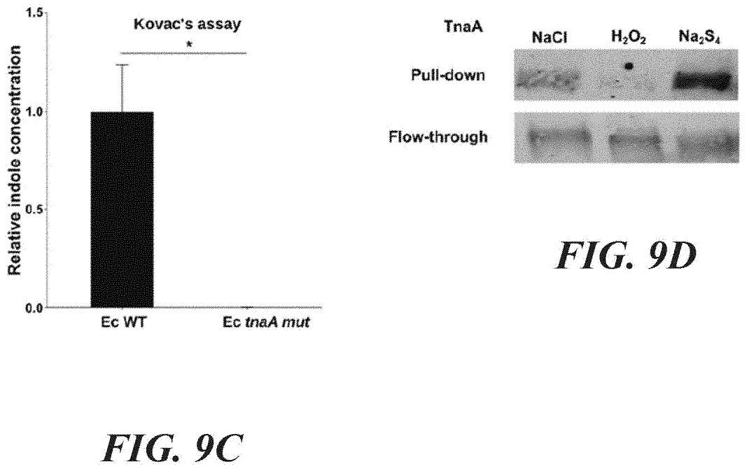

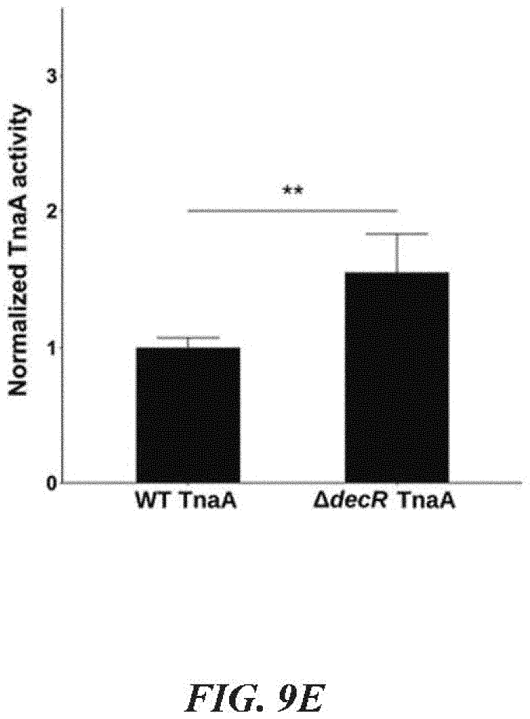

TnaA cysteine residues have been reported to be important for its enzymatic activity (Tokushige et al., 1989), as mutation of cysteine 298 results in inhibition of TnaA activity, due to a defect in homo-dimer formation (Kogan et al., 2009; Phillips and Gollnick, 1989). To study the effect of S-sulfhydration on TnaA activity, indole concentrations were measured by both Kovac's reagent and LC-MS/MS analysis of bacterial cultures. It was discovered that supplementing LB broth with cysteine or NaHS reduced indole concentrations in the supernatants (FIG. 8D-8E). Also, supporting sulfide's role in TnaA inhibition, ΔdecR E. coli had higher indole levels compared to WT E. coli when grown in LB supplemented with cysteine (FIG. 9A), and TnaA expression was similar under these conditions (FIG. 9B). To demonstrate that indole production was dependent on TnaA, TnaA activity was ablated by using an isogenic tnaA739::kan mutant (tnaA mut) and did not detect indole in the culture supernatant (FIG. 9C).

To further validate that S-sulfhydration inhibits TnaA activity in a direct manner, a reductionist approach was employed using purified E. coli TnaA. It was observed that incubation with disodium tetrasulfide (Na2S4), a poly-sulfide donor, led to TnaA S-sulfhydration (FIGS. 9D-9E) and reduced enzymatic activity by 60% in vitro (p<0.05, two-way ANOVA with post-hoc Tukey's test) (FIG. 8F). As an assay control, DTT was added, which should reduce S-sulfhydrated TnaA to its functional native form, and observed TnaA activity increased by 318% (p<0.001, two-way ANOVA with post-hoc Tukey's test). To provide a more physiological context for TnaA inhibition by cysteine-derived sulfide, the activity of TnaA purified from WT and ΔdecR E. coli cultures grown with cysteine was measured and found that TnaAΔdecR had higher activity (FIG. 8F). Collectively, these results support that S-sulfhydration of E. coli TnaA reduces its activity as measured by indole production from tryptophan, both in vitro and in bacterial cultures.

### Dietary Saa Modulate Cecal Indole Levels, Serum Indoxyl Sulfate Levels, and Kidney Function in a Mouse CKD Model

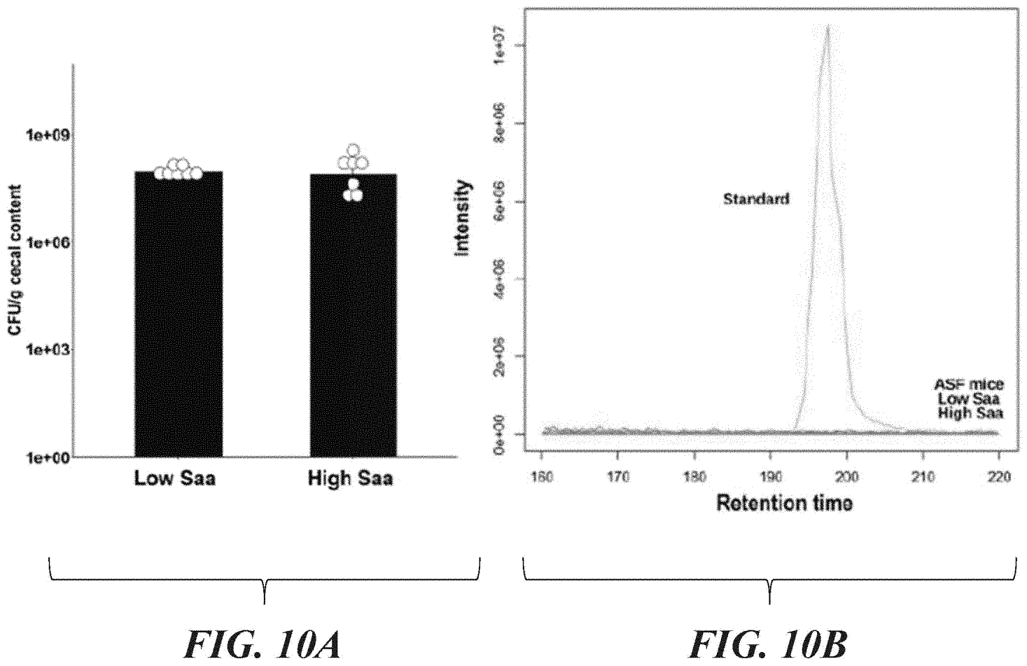

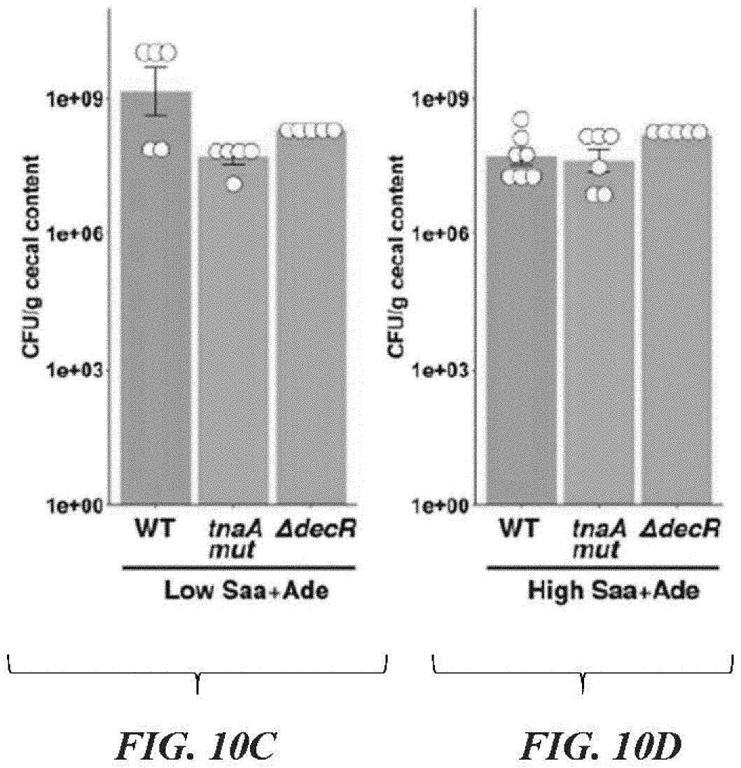

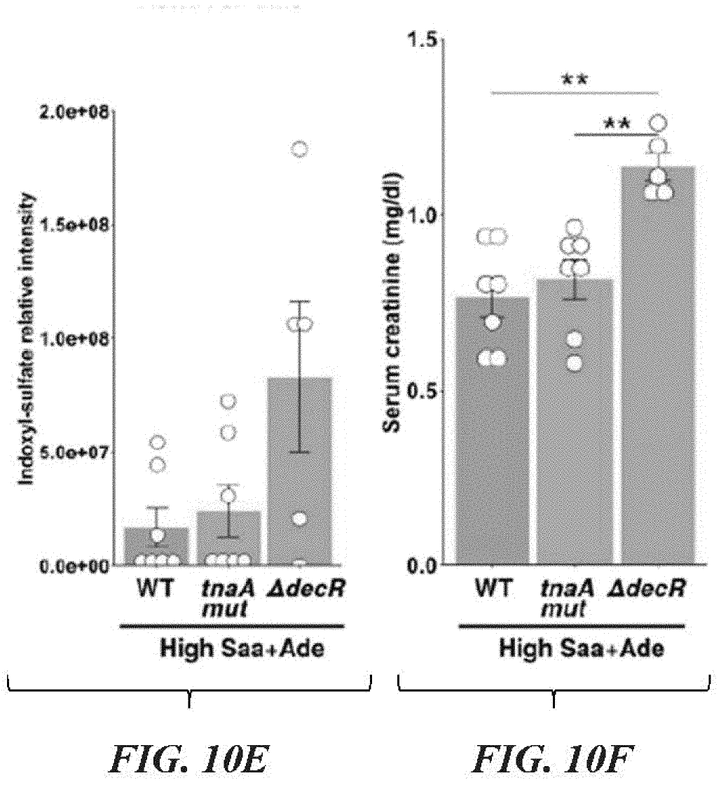

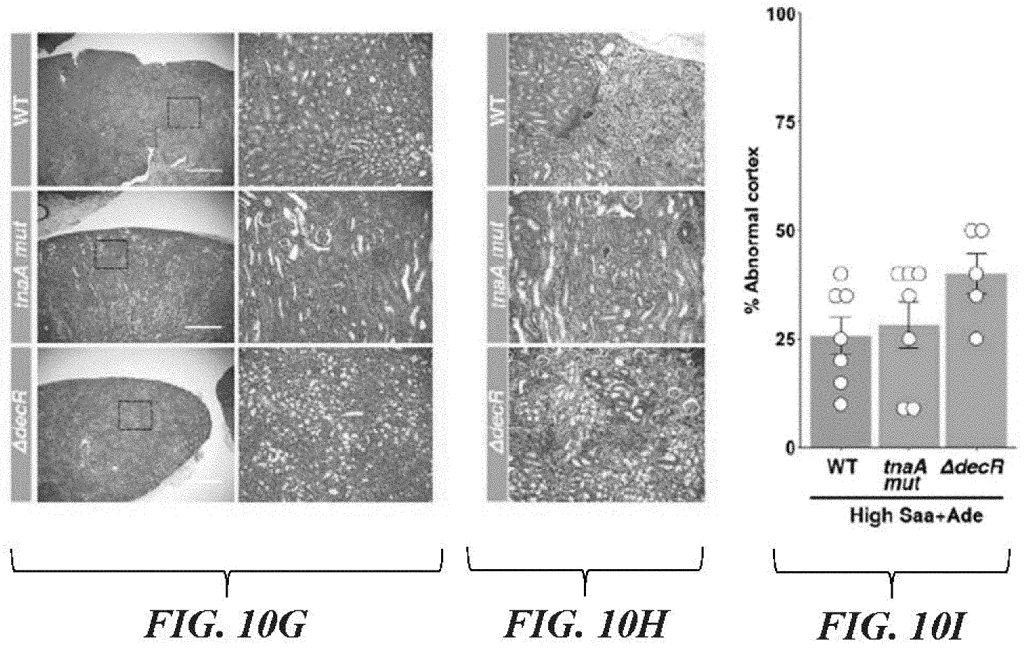

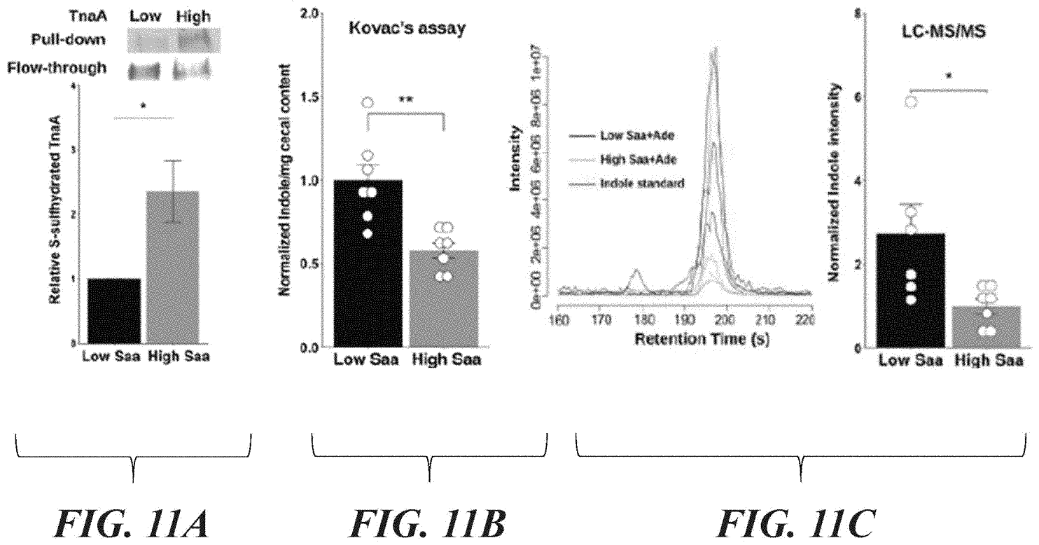

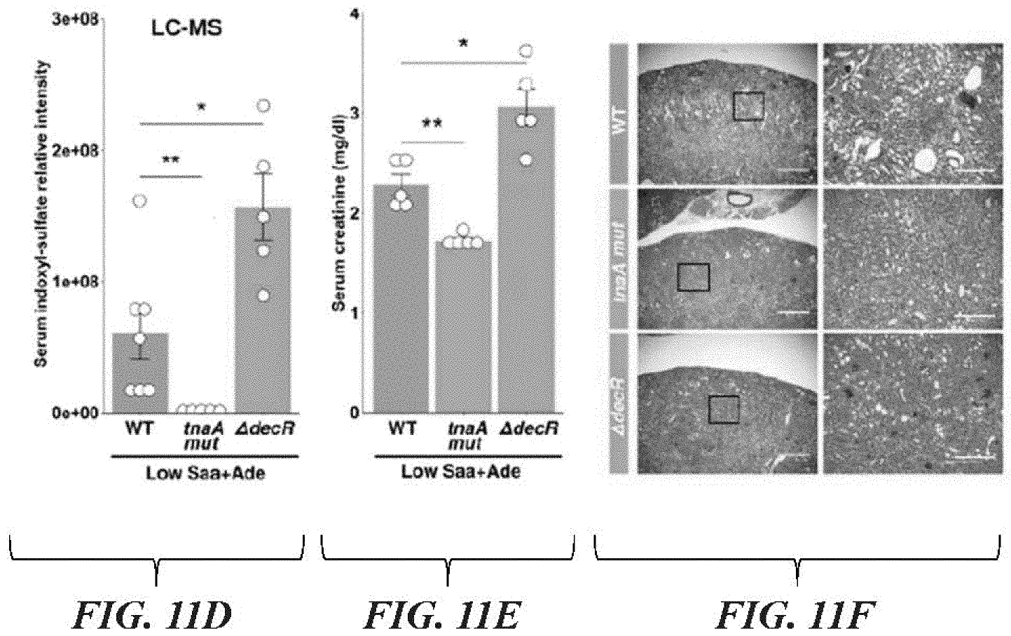

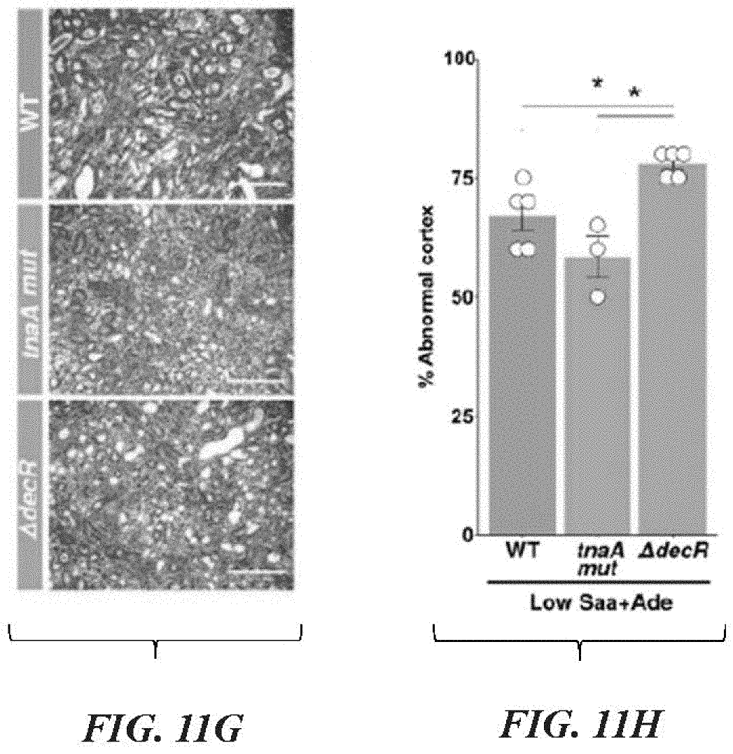

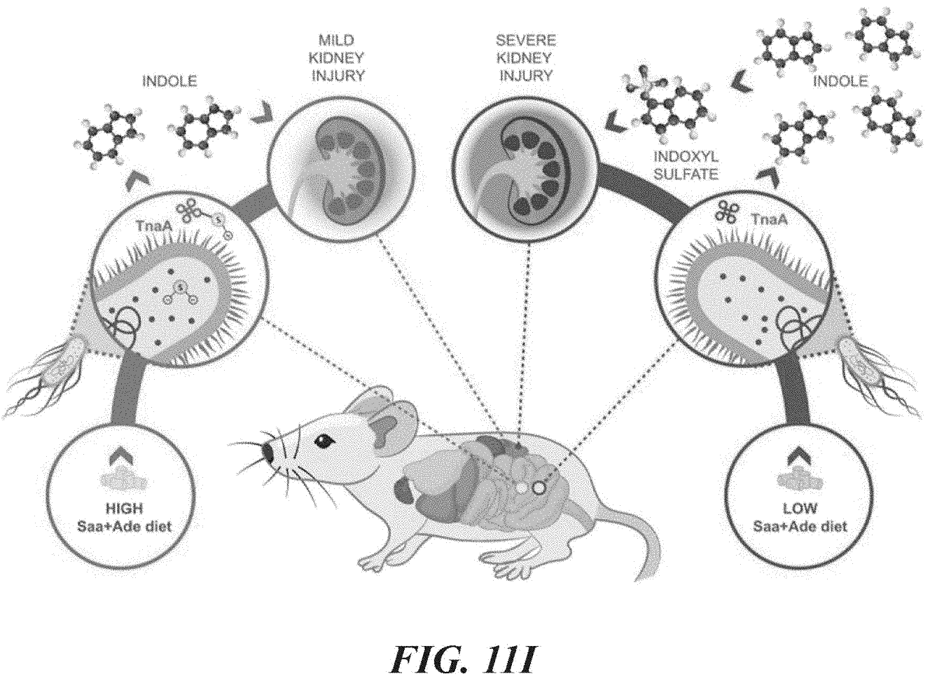

The detection of TnaA S-sulfhydration in vitro for both purified protein and TnaA from bacterial cell lysates and demonstrated that this modification inhibited its activity. Next, it was determined if this post-translational modification occurred within the gut in response to dietary Saa and resulted in measurable physiologic consequences for the host. ASF^{E.coli} mice were provided with the high and low Saa diets. While mice on the diets harbored similar levels of E. coli (FIG. 10A), higher TnaA S-sulfhydration was detected in the cecal contents of mice on the high Saa diet compared to those on the low (2.4-fold mean increase with high vs low Saa diet, p<0.05, Mann-Whitney U test) (FIG. 11A). None of the 8 ASF bacterial genomes encode a tnaA gene, and indoles could not be detected in their cecal contents using LC-MS/MS, implying that E. coli is the sole producer of indoles in this model (FIG. 10B). Taking advantage of that distinction, indole was measured in the cecal contents of ASF E. coli mice on the two diets, and found that mice on the high Saa diet had significantly lower indole levels demonstrating that high dietary Saa not only increased TnaA S-sulfhydration, but that this modification was sufficient to affect TnaA activity in vivo (FIG. 11B-11C). To strengthen the links between diet, microbial metabolism, and kidney damage, the CKD preclinical model was leveraged using the low Saa+Ade diet, in which the most renal injury was observed (FIG. 1), and the gnotobiotic ASF mice used previously (FIG. 5). The mice were colonized with either WT E. coli (ASF E. coli), or with one of two isogenic mutants, tnaA mut or ΔdecR (ASF tnaA mut and ASFΔdecR, respectively). Colonization of the three different E. coli strains was similar (FIG. 10C). Unlike in ASF^{E.coli} mice, no indoxyl sulfate was detected in the serum of ASFtnaA mut mice as there was no tryptophanase present within the gut microbiota. As E. coli ΔdecR is deficient in producing sulfide from cysteine, TnaA remains less S-sulfhydrated/more highly active in culture (FIGS. 8A and 9A).

Consistent with that observation, serum indoxyl sulfate from ASFΔdecR mice was increased relative to the sera levels observed from ASF E. coli mice (FIG. 11D). Mice colonized with WT E. coli had higher serum creatinine levels compared to mice colonized with the tnaA mut strain (FIG. 11E). Concomitant with the serum indoxyl sulfate levels, mice colonized with the ΔdecR strain had the highest serum creatinine levels (FIG. 11E). Histological findings of more severe tubulointerstitial damage, fibrosis, and cortical crystal deposition and more extensive parenchymal involvement mirrored the trends observed for indoxyl sulfate and creatinine for the E. coli ΔdecR vs WT E. coli (FIGS. 11F-11G). These mice were also examined on the high Saa+Ade diet. Consistent with prior observations (FIG. 5), ASF^{E.coli} mice on this diet demonstrated more mild phenotypes as compared to the low Saa+Ade diet, although ASFΔdecR mice had slightly increased serum creatinine compared to the parental and tnaA mut strains (FIG. 10D-10H). Collectively, these data both support that a dietary component can be metabolized by the microbiota to generate a post-translational modification of microbial proteins that affects host physiology and furnish mechanistic insight into how host-diet-microbiota interactions can contribute to prevalent disease states such as CKD.

### Summary of Results

The gut microbiota produce a myriad of diet-derived microbial metabolites that function in microbe-microbe and host-microbe interactions. Comprehensive efforts to decipher mechanisms mediating the physiological effects imposed by these metabolites are underway. However, currently knowledge is limited to a mere handful of such metabolite groups and their physiological effects (Postler and Ghosh, 2017). In this study, it was discovered that sulfide derived from bacterial metabolism of dietary Saa regulates E. coli indole production through inhibition of tryptophanase by S-sulfhydration. A dietary intervention that modulated Saa levels resulted in differential cecal indole and serum indoxyl sulfate levels. When investigated in the setting of a mouse CKD model, mice on a low Saa diet had a more severe phenotype exhibiting increased kidney damage and higher levels of serum creatinine that was dependent on TnaA activity and H₂S production (FIG. 11H). Therefore, this work, which builds upon prior observations that dietary Saa increase gut H₂S levels and that gut bacterial metabolism of tryptophan results in indole production (Devlin et al., 2016), reveals a mechanistic link between diet, microbial metabolism, and host physiology. More broadly, this work shows that a dietary component can be metabolized by the microbiota to generate a post-translational modification of microbial proteins that affects host physiology and furnish mechanistic insight into how host-diet-microbiota interactions can contribute to prevalent disease states such as CKD.

Over the past two decades, many studies have uncovered fascinating associations between specific bacterial species, microbiota compositions, or microbial metabolites with a range of host phenotypes (Brown and Hazen, 2015; Rooks and Garrett, 2016; Sharon et al., 2016). However, most of these studies focused on census-like surveys of the microbiome through 16S rRNA gene amplicon sequencing or whole genome shotgun metagenomics. Thus, scenarios in which there is no change in microbial composition, but rather in microbial activity, may have at times been overlooked by such census-like approaches. While metatranscriptomics have provided a window into functional changes within a community, these results demonstrate that at times delving even more deeply—beyond transcriptomics and even proteomics—to the effect of a single modification on one specific protein is necessary. Indeed, this work leverages a subtle dietary change, which does not result in microbial composition changes in the mouse CKD model, to show that production of indoles by E. coli is differentially affected by levels of sulfide endogenously produced by gut bacteria. Hence, these results emphasize not only the effect of bacterial metabolism on host physiology, but also potential microbe-microbe interactions driven by bacterial post-translational modifications mediated by host diet beyond the S-sulfhydration studied herein. Other well-studied diet-microbe molecules, such as short-chain fatty acids, might regulate bacterial or host phenotypes through acetylation of proteins (Ren et al., 2017), opening up a diet-microbe-host axis focused on microbiota protein post-translational modifications that heretofore has been underexplored.

Pre-dating symbiosis between micro-organisms and animals, sulfide was a key molecule for life. Sulfide predates oxygen as an electron acceptor, suggesting a potential role for S-sulfhydration in co-adaption. In support of this concept, several bacterial transcription factors regulating sulfur metabolism are affected by S-sulfhydration (Shimizu and Masuda, 2019). While S-sulfhydration of numerous mammalian proteins has been reported (Paul and Snyder, 2012), knowledge of bacterial S-sulfhydration is very limited and has employed non-quantitative mass-spectrometry approaches (Peng et al., 2017). This study provides a foundational survey of the E. coli S-sulfhydrome with quantitative TMT LC-MS3 analysis. Although the studies focused on TnaA S-sulfhydration herein, S-sulfhydration on the other 211 proteins identified may also have functional outcomes. The observation of enrichment of translation-related proteins in the S-sulfhydrome implies an interesting link between high sulfide conditions (e.g. cysteine toxicity) and translation regulation. However, further investigation is needed to evaluate this correlation.

While here the studies focused on S-sulfhydration, sulfide by itself has other physiological effects mediated through mechanisms, such as inhibition of cellular respiration and modulation of cellular sulfur redox potential. Therefore, it is not surprising that sulfide has been linked to renal function previously (Cao and Bian, 2016). The observations of diminished renal function on the low Saa+Ade diet compared to the high in GF and ASF mice (FIGS. 1A-1D and FIGS. 5A-5D), in which the studies did not detect cecal indoles, support the multifaceted role of Saa on kidney function. These findings also suggest that an endogenous microbiota-independent mechanism, such as Saa's induction of glutathione which can alleviate kidney injury in rats (Thielemann et al., 1990), may be at play. The observations and this published finding emphasize the complexity of CKD, which is influenced by various factors, including microbiota activity.

Indoxyl sulfate is a known uremic toxin, derived from oxidation of indole in the liver, and found at high concentrations in the plasma of CKD patients (Poesen et al., 2015). Since indole is derived from bacterial catabolism of dietary tryptophan, the microbiota is thought to play a role in regulating CKD patient blood indoxyl sulfate levels (Wikoff et al., 2009). The gnotobiotic CKD model results elucidate mechanisms underlying this disease process in humans, as cecal indole levels and serum indoxyl sulfate levels were higher in mice on the low Saa+Ade diet. Diet is a crucial aspect in managing CKD (Chen et al., 2019; Yang and Tarng, 2018). Not wishing to be bound by a particular theory, it was hypothesized that administration of TnaA inhibitors, such as sulfide donors, can help reduce gut indole levels and thus mitigate kidney damage. In support of this concept and its broad application, other gut bacteria, especially members of the Bacteroidetes phylum encode for TnaA homologs (Devlin et al., 2016), and a high degree of homology exists between bacterial TnaA alleles (FIG. 12). Overall, this study elucidates an interaction between diet, microbial metabolism, and kidney function, mediated by post-translational protein regulation. These findings might shed light on managing CKD and provide clinical approaches that target the microbiota and the enzymatic activities of its proteome to improve human health.

### Material and Methods

### Mice and Dietary Interventions

C57BL/6J (B6) mice were obtained from Jackson Laboratory and were housed in a barrier facility with constant ambient temperature of 24° C. and 12 h of day/night cycles. For gnotobiotic experiments, mice were housed at a gnotobiotic facility in semi-rigid isolators and experiments were conducted in individual ventilated cages. Routine qPCR analyses (using universal 16S rDNA primers (Hunter et al., 2002)) were performed on fecal samples and cage swabs to validate the gnotobiotic status of the animals. In order to generate mice that harbor the Altered Schaedler Flora (ASF) microbiota, germ-free (GF) mice were gavaged with cecal contents of ASF mice. Colonization was determined by qPCR as previously reported (30). Sulfur amino acid (Saa) diets were formulated based on the literature (31, 32) to represent edge cases of Saa consumption, with the following considerations. Human dietary cysteine and methionine consumption in western populations ranges between 0.03-0.06 g/kg body weight/d (33-35). Across human diets that varied in their protein consumption (44 g-140 g/day), levels specifically of cysteine have ranged between 0.01-0.04/g/kg/d (36) and these values have been deemed insufficient especially for the elderly, a population with an increased incidence of CKD. Notably, in humans, very high levels of met or cys are in the range >=6 g/kg/day, such levels raise concerns for contributing to homocysteinemia. Mindful of these data and with veterinary approval at our university, we formulated the diets employed in these studies (see TABLE 1 for the diet formulations) and manufactured by Research Diets, Inc. The lower Saa diet contains enough methionine to avoid the effects of methionine restriction (Cooke et al., 2018). 6-8 weeks old conventional or ASF mice were placed on an Saa diet and maintained on it until the experimental end-point. For generating ASF mice colonized with E. coli strains, 6-8 weeks old B6 ASF mice were gavaged with 5×107-5×108 colony forming units (CFU) of E. coli strains, and cecal colonization was confirmed at the end-point. For the preclinical CKD model, 0.2% adenine was added to the Saa diets and 6-8 weeks old B6 ASF or ASF E. coli mice were maintained on Saa+0.2% adenine (Saa+Ade) diets for 7 weeks (Jia et al., 2013). The diets were manufactured by Research Diets, Inc. Animal studies and experiments were approved and carried out in accordance with the National Institutes of Health guidelines for animal use and care.

### Bacterial Strains and Media

E. coli K-12 BW25113 strain were used in all the experiments (H₂S production, indole production and in vivo experiments). For technical reasons, E. coli K-12 MG1655 was used in the cloning process to generate ΔdecR and tnaA-his, and E. coli K-12 W3110 was used for expressing and purifying TnaA-His. Bacteria were grown in LB broth (Merck) at 37° C. with shaking (250 rpm) aerobically or without shaking anaerobically and where mentioned, LB was supplemented with L-cysteine (Sigma-Aldrich), sodium hydrosulfide (Sigma-Aldrich) and/or L-tryptophan (Sigma-Aldrich). For selection on LB+Chloramphenicol (Cm) agar plates, a concentration of 10 μg/ml Cm was used. The E. coli tnaA739::kan strain was obtained from Yale University Coli Genetic Stock Center (CGSC) as part of the Keio collection (Baba et al., 2006).

Genetic Manipulations and Cloning of E. coli

To generate the in-frame decR deletion mutant, 1000 bp up-stream and down-stream of decR coding sequence were amplified using 2 consecutive PCR reactions (list of primers, purchased from Sigma-Aldrich, TABLE 3) to construct a PCR product that contains only the first and last codons of decR. The PCR product was then digested with BamHI and NotI restriction enzymes, ligated into the pKOV plasmid (obtained from AddGene) and chemically transformed into E. coli MG1655. Bacteria were plated on LB+Chloramphenicol (Cm) plates and incubated at 30° C., due to the temperature sensitivity of the origin of replication (Link and Phillips, 1997). The pKOV-decR plasmid was extracted from colony PCR positive colonies and was sequenced by Sanger sequencing (Genewiz®) to verify the sequence of the insert. E. coli BW25113 strain was then transformed with the pKOV-decR plasmid and plated on LB+Cm plates at 30° C. overnight. Resistant colonies were inoculated into LB+Cm and grown at 42° C. overnight to force the integration of the plasmid into the bacterial genome. CmR colonies were picked and grown in LB without antibiotics for three passages at 30° C. to allow the excision of the plasmid. Finally, the bacteria were plated on salt-free LB plates containing sucrose, which should restrict the growth of bacteria that still contain the plasmid (and the sacB gene). Colony PCR was used to validated the deletion site. A similar procedure was used to replace the chromosomal tnaA copy with a tnaA-his allele, except that the initial PCRs were leveraged to sew the his-tag into the tnaA PCR product (TABLE 3).

TABLE 3
PRIMER SEQUENCES

| Primer | |
|---|---|
| Name | Sequence |
| TnaA-his A | ATATGGATCCTCTGGCGGTAGGTCTGTATG |
| | (SEQ ID NO: 1) |
| TnaA-his B | GTGGTGGTGGTGGTGGTGCTCGAGAACTTC |
| | TTTAAGTTTTGCGGTG (SEQ ID NO: 2) |
| TnaA-his C | CTCGAGCACCACCACCACCACCACTAATTA |
| | ATACTACAGAGTGGCTATAAGG |
| | (SEQ ID NO: 3) |
| TnaA-his D | ATATGCGGCCGCCCAAACACGATCACAAAG |
| | GAG (SEQ ID NO: 4) |
| DecR-del A | ATATGGATCCGCGTATTTGATTACCGGCAAC |
| | (SEQ ID NO: 5) |
| DecR-del B | GTCCTGACCTGATTCTGGATATTTATTCTAA |
| | CATAGCCCTTCCACAGAGAA |
| | (SEQ ID NO: 6) |
| DecR-del C | TTCTCTGTGGAAGGGCTATGTTAGAATAAA |
| | TATCCAGAATCAGGTCAGGAC |
| | (SEQ ID NO: 7) |
| DecR-del D | ATATGCGGCCGCTGCACCACCATCCAACTACC |
| | (SEQ ID NO: 8) |

### Protein Extraction

Bacterial cultures were grown in LB medium until mid-log growth (OD600 nm=0.5-0.7) and then bacteria were pelleted by centrifugation at 4000 rpm for 10 min at 4° C. When indicated, the bacterial cultures where supplemented with 5 mM cysteine or 1 mM NaHS after 3 h of growth, allowed to grow for an additional 1 h and then pelleted. The supernatants were removed and the pellets were washed once in cold PBS to remove extracellular proteins. Washed bacterial pellets were resuspended in 1 ml of cold lysis buffer (100 mM Tris-HCl pH 7.1, 150 mM NaCl, 1 mM EDTA, 0.5% deoxycholate and 0.5% Triton X-100) and transferred into a 2 ml tube containing 300 μl zirconium beads (20 micron). The lysis buffer was de-gassed using argon to reduce loss of S-sulfhydration signal by oxidation and supplemented with Complete Ultra protease inhibitor cocktail (Roche) and Phospho-Stop phosphatase inhibitor (Roche). The tubes were then placed in a bead-beater for 2 min and then centrifuged for 10 min at 14,000 rpm at 4° C. The supernatant was transferred to a new 1.5 ml tube and immediately frozen in liquid N2 to reduce the possibility of cysteine residues oxidation. Protein quantification was performed using a BCA assay (Thermo Fisher) on a 1:10 diluted sample. For mouse cecal samples, ceca of 3 mice on low or high Saa diet were pooled and resuspended in cold TBS buffer to generate one sample under each condition. The samples were gently vortexed for 10 min to homogenize the solution. Then, the samples were centrifuged for 3 min at 200 g to remove undigested food particles and the supernatant was transferred to a new 1.5 ml tube and centrifuged for 10 min at 14000 rpm at 4° C. The supernatant was discarded and the pellets were resuspended in 0.6 ml cold lysis buffer and transferred to a 2 ml tube with 300 μl of zirconium beads (20 micron) and processed similarly to the bacterial samples. For analyzing TnaA-His S-sulfhydration, bacterial cultures of E. coli tnaA-his W3110 strain were grown in LB for 3 h and then 5 mM cysteine was added for 1 h. Protein was extracted as mentioned above, purified using the His-Spin protein miniprep (Zymo Research®) and desalted using Zeba micro-columns (Thermo Fisher®) into 100 mM TEAB solution. TnaA purity was determined by Coomassie blue staining.

### Pull-Down of S-Sulfhydrated Proteins

Frozen lysates were thawed on ice and 1-2 mg of protein in 125 μl were incubated with occasional stirring at room temperature for 1 h with freshly prepared 100 μM maleimide-PEG2-biotin to label the thiol groups. As needed, samples were concentrated using Amicon Ultracel 3K™ nanosep columns (Millipore®). To remove the unbound maleimide molecules, the samples were desalted using Zeba micro-columns (Thermo Fisher®) into binding buffer (50 mM Tris-HCl pH 7.5, 0.1% SDS, 150 mM NaCl, 1 mM EDTA and 0.5% Triton X-100). Sample volume was adjusted to 250 μl and samples were incubated overnight (16 h) at 4° C. with 50 μl of pre-washed high-capacity binding streptavidin-agarose beads (Thermo Fisher). The following day the samples were moved onto a micro-column (Thermo Fischer) and centrifuged for 1 min at 1000 g, the flow-through was collected and labeled as flow-through. Then the beads were washed (all washes were 250 μl at 1000 g for 1 min) on-column 3 times with wash buffer A (50 mM Tris-HCl pH 7.5, 150 mM NaCl, 0.5% Triton X-100), followed by 3 washes with wash buffer B (50 mM Tris-HCl pH 7.5, 600 mM NaCl, 0.5% Triton X-100) and finally one wash with elution buffer (50 mM Tris-HCl, 150 mM NaCl), before incubation with 500 μl elution buffer supplemented with 20 mM dithiothreitol (DTT) for 30 min and then eluted by 1 min centrifugation at 1000 g. The pull-down samples were concentrated using Amicon Ultracel 3K™ nanosep (Millipore®) by centrifuging at max speed for 10 min or until the samples contained 25-30 μl. The beads were resuspended in 300 μl elution buffer, boiled for 10 min and collected as beads-bound fractions. Pull down fractions were visualized by either Coomassie blue (Bio-Rad®) or silver stain (Thermo Fisher®).

### FASP On-Column and TMT Labeling

On-column protein digestion and labeling were performed using FASP digestion kit (Expedeon®) following the iFASP protocol (McDowell et al., 2013). To increase the recovery of peptides, micro-columns (10 kDa MWCO) and collection tubes were incubated overnight in 5% Tween-20 and soaked twice (10 min each) with sterile double distilled water (DDW). Then the micro-columns were washed twice with 500 μl of mass-spec grade water (Roche®). 3 μl of 200 mM TCEP were added to 30 μl of pull-down protein sample in 1.5 ml tube and incubated for 1 h at 55° C. Then, the samples were cooled to room temperature and 200 μl of 8M urea (in 0.1M Tris-HCl, pH 8.5) were added. The samples were transferred to 10 kDa MWCO micro-columns and spun at 14000 g for 15 min. The membranes were washed with 200 μl urea 8M and then 100 μl of 0.05M iodoacetamide in 8M urea solution were added. The tubes were shaken for 1 min at 600 rpm and then incubated at room temperature in the dark for 20 min, before being spun again at 14000 rpm for 15 min, washed twice with 200 μl 8 M urea solution pH 8.5, and washed 3 times with 100 μl of 100 mM TEAB solution. 75 μl of 1:50 dilution of Trypsin (Thermo Fisher®) at 1 μg/μl in 50 mM acetic acid with 0.02% ProteaseMAX (Promega®) were added to the micro-columns, which were then incubated at 37° C. at 600 rpm for 16 h. The tubes were sealed with parafilm to avoid drying of the membranes. Next, TMT reagents (Thermo Fisher®) were equilibrated to room temp and dissolved in 41 μl of anhydrous acetonitrile for 5 min with occasional vortexing. Then, each TMT label was added to a micro-column and incubated at room temperature in the dark at 600 rpm for 1 h. The reactions were quenched by adding 8 μl of 5% hydroxylamine and room temperature incubation for 30 min at 600 rpm. Labeled peptides were eluted by passing 40 μl of 100 mM TEAB over the columns 3 times followed by 50 μl of 0.5M NaCl solution. The TMT labeled channels (3 repeats of WT E. coli, WT E. coli without DTT elution and ΔdecR lysate, as well as a reference channel, made by combining equal volumes of the 9 samples prior to TMT labeling), were combined into one tube and dried using a speed-vac. The dried pooled sample was resuspended in 1 ml 1% trifluoroacetic acid (TFA) solution and incubated for 30 min with shaking at 600 rpm and then dried again using a speed-vac. The pooled sample was then resuspended in 300 μl of 0.1% TFA and fractionated using the Pierce High pH Reversed-Phase Peptide Fractionation Kit (Thermo Fisher) into 5 fractions (10%, 15%, 20%, 25% and 50% acetonitrile), dried in a speed-vac and resuspended in 0.1% formic acid.

### Metabolite Extractions for LC-MS/MS Analysis of Indole and Indoxyl-Sulfate

Extraction of metabolites from cecal samples was performed similar to (Jin et al., 2014; Sellick et al., 2010) by collecting cecal contents into empty pre-weighed 2 ml tubes; and after weighing the contents, 1.5 ml of cold methanol/chloroform (2:1 v/v) solution were added. The samples were vortexed and homogenized with a wide-bore tip on ice and then centrifuged for 10 min at 15,000 g at 4° C. The supernatant was transferred to a new 5 ml tube, 0.6 ml of ice-cold double-distilled water were added and the samples were vortexed and centrifuged at 15,000 g for 5 min at 4° C. to obtain phase separation. The upper aquatic phase and lower organic phase were collected carefully without dispersing the proteinaceous interface into 1.5 ml tubes and kept in −80° C. until LC-MS/MS analysis. For bacterial culture indole measurements, overnight bacterial cultures were diluted 1:100 and grown for 3 h at 37° C. in LB, then either mock, 5 mM L-cysteine or 1 mM NaHS were added for 1 h. Bacterial cultures were harvested by centrifugation, the supernatants were filter-sterilized through 0.2 μm filters and 225 μl of supernatant were transferred to a new 1.5 ml tube. Then, 25 μl of 10 mM L-tryptophan was added and the samples were incubated 1 h at 37° C., before 250 μl of 20% TCA were added and the samples were incubated on ice for 15 min to precipitate proteins. The samples were then centrifuged for 10 min at 4° C. and the supernatants were kept at −80° C. until LC-MS/MS analysis. For serum samples, mouse blood was collected into serum separator tubes (BD) tubes, inverted 5 times and allowed to clot for 30 min at room temperature. Then, samples were centrifuged for 15 min at 1300 g at 4° C. and the serum layer was carefully removed into a new 1.5 ml tube without disturbing the buffy coat layer. The samples volume was adjusted to 160 μl with PBS, 40 μl of trichloroacetic acid (TCA) were added to 20% final TCA concentration and samples were incubated on ice for 15 min to precipitate proteins. The samples were then centrifuged at max speed for 10 min at 4° C. and the supernatants were transferred to new 1.5 ml tubes and kept at −80° C. until LC-MS/MS analysis.

### Mass Spectrometry Analysis

The TMT fractions were analyzed by LC-MS3 on an Orbitrap Fusion™ Lumos™ Tribrid™ mass spectrometer. Labelled peptide samples were analyzed with an LC-MS3 data collection strategy (McAlister G C et al (2014) Anal. Chem. 86:7150-8) on an Orbitrap Fusion mass spectrometer (Thermo Fisher Scientific) equipped with a Thermo Easy-nLC 1200 for online sample handling and peptide separations. Resuspended peptide from previous step was loaded onto a 100 μm inner diameter fused-silica micro capillary with a needle tip pulled to an internal diameter less than 5 μm. The column was packed in-house to a length of 35 cm with a C18 reverse phase resin (GP118 resin 1.8 μm, 120 Å, Sepax Technologies®). The peptides were separated using a 180 min linear gradient from 6% to 35% buffer B (90% ACN+0.1% formic acid) equilibrated with buffer A (5% ACN+0.1% formic acid) at a flow rate of 500 nL/min across the column. The scan sequence for the Fusion Orbitrap began with an MS1 spectrum (Orbitrap analysis, resolution 120,000, scan range of 350-1350 m/z, AGC target 1×106, maximum injection time 100 ms, dynamic exclusion of 60 seconds). The “Top10” precursors were selected for MS2 analysis, which consisted of CID (quadrupole isolation set at 0.5 Da and ion trap analysis, AGC 2.5×104, Collision Energy 35%, maximum injection time 150 ms). The top ten precursors from each MS2 scan were selected for MS3 analysis (synchronous precursor selection), in which precursors were fragmented by HCD prior to Orbitrap analysis (Collision Energy 55%, max. AGC 2×105, maximum injection time 150 ms, resolution 50,000, and isolation window set to 1.2-0.8). E. coli TnaA-His samples were analyzed at the Mass Spectrometry and Proteomics Resource Laboratory. TnaA-His was not reduced and/or alkylated to preserve the state of native cysteine PTMs. LC-MS/MS was performed on a Orbitrap Elite™ Hybrid Ion Trap-Orbitrap Mass Spectrometer (Thermo Fischer®, San Jose, Calif.) equipped with WATERS™ Aquity nano-HPLC. Peptides were separated onto a 100 μm inner diameter microcapillary trapping column packed first with approximately 5 cm of C18 Reprosil resin (5 μm, 100 Å) followed by analytical column ˜20 cm of Reprosil resin (1.8 μm, 200 Å). Separation was achieved through applying a gradient from 5-27% ACN in 0.1% formic acid over 90 min at 200 nl min-1. Electrospray ionization was enabled through applying a voltage of 1.8 kV using a home-made electrode junction at the end of the microcapillary column and sprayed from fused silica pico tips (New Objective, MA). The mass spectrometry survey scan was performed in the Orbitrap in the range of 395-1,800 m/z at a resolution of 6×104, followed by the selection of the twenty most intense ions (TOP20) for CID-MS2 fragmentation in the Ion trap using a precursor isolation width window of 2m/z, AGC setting of 10,000, and a maximum ion accumulation of 200 ms. Singly charged ion species were not subjected to CID fragmentation. Normalized collision energy was set to 35 V and an activation time of 10 ms. Ions in a 10 ppm m/z window around ions selected for MS2 were excluded from further selection for fragmentation for 60s. The same TOP20 ions were subjected to HCD MS2 event in Orbitrap part of the instrument. The fragment ion isolation width was set to 0.7 m/z, AGC was set to 50,000, the maximum ion time was 200 ms, normalized collision energy was set to 27V and an activation time of 1 ms for each HCD MS2 scan. Metabolite samples were analyzed for indole and indoxyl sulfate content. Quantification of indole by LC/MS/MS were carried out on a Thermo Scientific Dionex UltiMate 3000™ UHPLC coupled to a Thermo Q Exactive Plus™ mass spectrometer system (Thermo Fisher Scientific, Inc., Waltham, Mass.) equipped with an APCI probe for the Ion Max API source. Data were acquired with Chromeleon Xpress™ software for UHPLC and Thermo Xcalibur™ software version 3.0.63 for mass spectrometry and processed with Thermo Xcalibur Qual Browser™ software version 4.0.27.19. 3 μL sample was injected onto the UHPLC including an HPG-3400RS binary pump with a built-in vacuum degasser and a thermostated WPS-3000TRS high performance autosampler. An Xterra™ MS C18 analytical column (2.1×50 mm, 3.5 μm) from Waters Corporation® (Milford, Mass.) was used at the flow rate of 0.3 mL/min using 0.1% formic acid in water as mobile phase A and 0.1% formic acid in methanol as mobile phase B. The column temperature was maintained at room temperature. The following gradient was applied: 0-6 min: 20-100% B, 6-8 min: 100% B isocratic, 8-8.1 min: 100-30% B, 8.1-11.1 min, 20% B isocratic. The MS conditions were as follows: positive ionization mode; PRM with the precursor→product ion PRM transition, m/z 118.0651 ([M+H]+)→91.0542 ([C7H7]+); normalized collision energy (NCE), 105; resolution, 70,000; AGC target, 2e5; maximum IT, 220 ms; isolation window, 1.8m/z; spray voltage, 5000V; capillary temperature, 250° C.; sheath gas, 28; Aux gas, 5; probe heater temperature, 363° C.; S-Lens RF level, 55.00. A mass window of ±5 ppm was used to extract the ion. Indole was considered detected when the mass accuracy was less than 5 ppm and there was a match of isotopic pattern between the observed and the theoretical ones and a match of retention time between those in real samples and the standard. Isotope labeled (13C) and native standards of indole and indoxyl sulfate were obtained from Toronto Research Chemicals.

### Computational Analysis of Mass-Spectrometry Data

Thermo Fisher RAW files were converted to mzML files using ProteoWizard™ (Chambers et al., 2012). The MS/MS spectra were searched against a target and decoy database comprising the Uniprot E. coli K-12 proteome and a list of frequent mass-spectrometry contaminants using MSGF+ (v2017.08.23) with the following parameters -protocol 4 -t 10 ppm -mod MSGF_mod.txt -tda 0 -addFeatures 1 -maxCharge 4 (Kim and Pevzner, 2014). The modification file included static alkylation of cysteine (57.02146 Da), static TMT labeling of lysine residues and N-termini of peptides (229.162932 Da), and variable oxidation of methionine (15.99491 Da). Post-search peptide filtering was performed using Percolator (v3.1.2) (The et al., 2016) and the output psms files was manually filtered to include only psms with q value <=0.01 and pep score <=0.05. The filtered psms file was converted to pepXML using OpenMS (v2.2.0) IDfileConverter (Röst et al., 2016) and then TMT MS3 reporter ion quantification was performed using pyQuant (v2.1)(Mitchell et al., 2016). Finally, peptides that had an intensity value of 0 in the reference channel or had a non-zero value only in the reference channel were removed, and proteins that were identified by only one peptide or mapped to contaminants were discarded. Statistical analysis was performed using Kruskal-Wallis and 5% false discovery rate (FDR) in R. For the analysis of TnaA S-sulfhydration, raw data were submitted for analysis in Proteome Discoverer 2.2 (Thermo Scientific) software. Assignment of MS/MS spectra was performed using the Sequest HT algorithm by searching the data against a protein sequence database including all entries from the E. coli proteome database as well as other known contaminants such as human keratins and common lab contaminants. Sequest HT searches were performed using a 20 ppm precursor ion tolerance and requiring each peptides N-/C termini to adhere with Trypsin protease specificity, while allowing up to two missed cleavages. A MS2 spectra assignment false discovery rate (FDR) of 1% on both protein and peptide level was achieved by applying the target-decoy database search. Visualization of peptide-match spectra was performed using SearchGUI (v3.3.15) (Barsnes and Vaudel, 2018) and PeptideShaker™ (v1.16.40) (Vaudel et al., 2015). Indole and indoxyl sulfate analyses were performed using the xcms (3.8.1) package in R.

### Western Blot Analyses

Equal volumes of pull-down or flow-through samples were incubated at 70° C. for 15 min with loading buffer. Samples were run on 10% Mini-PROTEAN® TGX™ Precast Protein Gels (Bio-Rad) with Chameleon® Duo Pre-stained Protein Ladder (LiCOr®) in Tris-Glycine-SDS run buffer. Proteins were then transferred to an Amersham Protran 0.45 μm nitrocellulose membrane in a Tris-Glycine transfer buffer for 1 h at 20 V at room temperature. The membranes were blocked using 1:2 dilution of Odyssey Blocking PBS Buffer (LiCOr®) in PBS for 1 h at room temperature. Membranes were then incubated with primary antibodies in blocking buffer+0.2% Tween-20 overnight. Following 5 washes of PBS+0.2% Tween-20 (5 min each), the membranes were incubated for 1 h in blocking buffer+0.2% Tween-20 with secondary antibodies conjugated to a fluorophore (LiCOr®). The membranes were washed again for 5 times with PBS+0.2% Tween-20 and then 2 more times with PBS, before being imaged on a LiCOr Odyssey CLx™ machine. Images were analyzed and quantified using ImageJ2 software (Rueden et al., 2017).

### Colorimetric Indole Measurement Using Kovac's Reagent

E. coli cultures were grown in 10 ml LB at 37° C. at 250 rpm for 3 h and then, for the treatment groups, 5 mM L-cysteine or 1 mM NaHS were added and the cultures were allowed to grow for an additional 1 h before cells were harvested by centrifugation at 14000 rpm for 10 min at room temperature. Then the supernatants were filter sterilized through 0.2 μm filters and 1 mM of L-tryptophan was added, and the supernatants were incubated at 37° C. for 1 h. Then 250 μl of 20% w/v TCA was added to 250 μl of supernatant and kept on ice for 15 min to precipitate proteins. The samples were centrifuged at 14000 rpm for 10 min, the supernatant was moved to a new 1.5 ml tube and 500 μl of Kovac's reagent (Sigma-Aldrich) were added. The samples were vortexed and incubated at 37° C. for 30 min, before the top 200 μl layer was moved to a 96-well plate, and OD530 nm was read. Indole (Sigma-Aldrich) serial dilutions were analyzed for generation of a standard curve. For cecal samples, cecal content was collected into pre-weighed 1.5 ml tubes containing 750 μl 70% EtOH. The samples were homogenized using vortexing and wide-bore tips and then incubated at 70° C. for 10 min. After 20 min centrifugation at max speed at 4° C., 150 μl of supernatant were added to 150 μl of Kovac's reagent, incubated for 30 min at room temperature and absorbance at OD530 nm was measured.

### In Vitro Tryptophanase Assays

E. coli apo-tryptophanase (Sigma-Aldrich) was resuspended in 1 ml 100 mM potassium phosphate pH 8 buffer, aliquoted and kept at −20° C. 5 μl of apo-tryptophanase were added to 120 μl of 100 mM potassium phosphate pH 8 buffer with 1 mM pyrodxal-5-phosphate (PLP) and various treatments (NaCl, NaHS, L-cysteine, DTT or Na2S4) and incubated for 45 min at 37° C. Then 125 μl of 100 mM potassium phosphate with 5 mM L-tryptophan were added and the samples were incubated for 1 h at 37° C. Then 250 μl of 20% TCA were added to the samples, followed by a 15 min incubation on ice to precipitate TnaA. Samples were centrifuged for 10 min at max speed, the supernatant was transferred to a new 1.5 ml tube and 500 μl of Kovac's reagent were added, followed by vortex and 30 min room temperature incubation, before absorbance of the top layer was measured at OD530 nm. For the WT and ΔdecR TnaA activity assay, TnaA was purified from 10 ml bacterial cultures grown for 3 h in LB and then supplemented with 5 mM L-cysteine for 1 h, using the His-Spin protein miniprep kit (Zymo research). The purified protein was diluted 1:10 in 250 μl of 100 mM potassium phosphate pH 8 buffer with 1 mM PLP and 5 mM L-tryptophan and incubated for 1 h at 37° C., further processing was performed as mentioned above. TnaA activity was normalized to purified protein concentration, measured using BCA assay kit (Thermo Fisher).

### Serum Creatinine Measurements

Mouse serum was extracted as mentioned above and creatinine levels were measured using the Serum Creatinine Colorimetric Assay Kit (Cayman Chemical) in duplicates with a standard curve, following the manufacturer's protocol.

### Cecal DNA Extraction and RT-qPCR Analysis

Mouse cecal contents were collected into 1.5 ml tube and flash frozen. Upon thawing, cecal contents were resuspended in lysis buffer (100 mM Tris-HCl pH 8.0, 15 mM EDTA and 2% SDS) and transferred to a 2 ml tube containing 300 μl zirconium beads (20 micron) and 500 μl of TE-saturated phenol (Sigma-Aldrich). The tubes were then placed in a bead-beater for 2 min and centrifuged for 10 min at 14,000 rpm at 4° C. The aqueous phase was transferred to a new 1.5 ml tube and an equal volume of phenol:chloroform solution was added. The samples were vortexed and centrifuged for 2 min at max speed at room temperature. This process was repeated 2 more times. Then the aqueous phase was moved to a new tube and 2 volumes of 100% EtOH and 1/10 volume of NaOAc pH 5.2 were added. The tubes were inverted several times and incubated at −20° C. for 1 h, before being centrifuged for 20 min at max speed at 4° C., and washed once in cold 70% EtOH. Finally, after air-drying, the samples were resuspended with 100 μl of sterile water and DNA concentration was determined using a photospectrometer machine. For RT-PCR analysis, 50 ng of cecal DNA were taken to a reaction with 10 μl of SYBR green (KAPA SYBER FAST) and the appropriate primers (Table 3). RT-PCR analysis was performed on a Applied Biosystems Stratagene MX3005P machine. The relative abundance of each ASF bacterium was determined by 2-ΔCt=(ASF bacterium Ct—Total 16S rRNA Ct).

Bacterial 16S rDNA Amplicon Library Generation and Sequencing

The procedures in this section were done in a biological hood to minimize potential contamination and were based on the Earth Microbiome Project protocol (Walters et al., 2016). 50 ng of cecal contents extracted DNA were taken to a PCR reaction using the Thermo Fisher Platinum Hot Start PCR Master Mix (cat. no. 13000014) according to the reagent protocol. Forward (10 μM) and reverse (1.3 μM) primers were used (see TABLE 3 for primer sequences) to amplify the V4 region of the 16S rRNA gene. For each sample the reverse primer contains a unique 12 bp Golay barcode. Each sample was amplified in triplicate with a 25 μl reaction volume per reaction in 96-well plates. Sterile water and E. coli genomic DNA served as negative and positive controls, respectively. The PCR reaction started with 3 min of 94° C., then 35 cycles of 45s 94° C., 60s 50° C. and 90s 72° C., followed by 10 min of 72° C. After amplification, the triplicate reactions were pooled and amplicons were purified using AMPure magnetic beads. DNA concentration was determined by dsDNA broad range assay kit (Thermo Fisher®) and a sample of several libraries was run on an agarose gel to visualize the specific amplicon. The libraries were pooled so that the final DNA concentration was 50 ng/μl and each library had an equal abundance. DNA sequencing was performed on an Illumina Mi-Seq machine at the bio-polymer core of HMS using the Mi-Seq V2 kit with a 250 bp paired-end reads. Raw sequences were deposited in NCBI SRA databank under the bioproject accession PRJNA603373.

Analysis of Bacterial 16S rRNA Gene Amplicon Sequences

A 16S rRNA gene amplicon sequence analysis was based on the suggested standard operating protocol by Langille et al. (Comeau et al., 2017) using the microbiome-helper wrapper. Briefly, fastq files were obtained for each library with a median read count of 73,905 250-bp paired-end reads and quality of reads was checked using FastQC (v0.11.5). According to the quality report, the reads were trimmed using the fastx-toolkit to keep only high-confidence base calls. Reads were stitched using PEAR (Zhang et al., 2014), converted to fasta format and chimeric reads were filtered out using VSEARCH (Rognes et al., 2016). Operational taxonomic units (OTUs) were picked using QIIME V1.9 (Caporaso et al., 2010) using the sortmerna program (Kopylova et al., 2012) and OTUs with fewer than 0.1% of the reads were excluded as low-confidence OTUs. Finally, the number of reads in each library was rarefied to the lowest library size. α-diversity and β-diversity (weighted Unifrac PCOA) analyses were conducted using the phyloseq R package v1.30 (McMurdie and Holmes, 2013) on Rstudio v1.25, as well as visualization of taxonomic compositions. Specific OTU differences between the two diets were analyzed using the phyloseq R package, LEfSe (Segata et al., 2011) and MaAsLin (Morgan et al., 2012) algorithms using caging as a cofounding variable. The python program STAMP was also used to visualize and analyze data (Parks et al., 2014).

### Meta-Analysis of CKD Patients Stool Microbiome Datasets

Sequence data of 16S rRNA gene amplicon sequencing of CKD patients stool samples (Xu et al., 2017 and unpublished) was downloaded from NCBI (accessions PRJEB9365 and PRJEB5761, respectively). 16S rRNA gene amplicon data was processed as described above. Metagenomic data of unpublished CKD patient stool samples were downloaded from NCBI (accession PRJNA449784) and analyzed by FastQC (v0.11.5), followed by trimming low quality reads and reads that map to the human genome using KneadData (v0.7.2). Next, the filtered reads were used as input for the HUManN2 program (v2.8.1) (Franzosa et al., 2018), yielding 3 matrices of gene families, metabolic pathways coverage and metabolic pathways abundance, stratified by bacterial species. For E. coli abundance analysis, the mean sum-normalized percentage of reads mapping to an E. coli gene was calculated and patients without E. coli mapped reads were removed from the analysis and then square root transformation was used to normalized the data. PhyloChip data from (Vaziri et al., 2013) was kindly provided by Dr. Vaziri and analyzed using R and the EnhancedVolcano™ package (v1.4.0).

### H₂S Measurements

Lead acetate paper was prepared by incubating pre-cut Whatman paper in 20 mM lead acetate solution for 20 min at room temperature and then was dried for 20 min at 110° C. and kept in the dark (Hine and Mitchell, 2017). For bacterial cultures H₂S production, overnight cultures of E. coli strains were diluted to OD600 nm of 0.05 in 200 μl of LB supplemented with various concentration of L-cysteine in a 96-well plate in triplicate. The plate was tightly covered with lead acetate paper and incubated at 37° C. for 7 h. The lead acetate papers were then scanned and densitometry analysis was performed using ImageJ2 (Rueden et al., 2017). For cecal contents, mouse cecal content was collected into pre-weighed 1.5 ml tubes with 300 μl of PBS, weighed, homogenized by vortex and pipetting. 200 μl were plated in 96-well plates in duplicate and analyzed by lead acetate sulfide assay as described above. For the colorimetric detection of sulfide by the methylene blue method (Florin, 1991; Moore et al., 1998), cecal contents were collected directly into pre-weighed 1.5 ml tubes with 200 μl of 1N NaOH to trap free sulfide ions in the S2-non-volatile form, on ice. The samples were homogenized with a wide bore pipette tip (10-20% cecal slurry). The samples were centrifuged at max speed at 4° C. for 10 min and in parallel a H₂S standard curve in 1N NaOH was prepared. After centrifugation, 150 μl of supernatant were transferred to a new tube and 200 μl of DPD/FeCl3 reagent (43 mM N,N-dimethyl-p-phenylenediamine sulfate and 148 mM FeCl3 in 4.2M HCl) were added. The samples were vortexed briefly and incubated for 20 min at 37° C., thereafter they were centrifuged for 4 min at max speed and the supernatants were transferred to a 96-well plate to determine absorbance at OD670 nm. The pellet from the initial centrifugation was also used to determine bound sulfide levels, as it was washed with 1N NaOH and then resuspended in 200 μl of 2% zinc acetate solution, pH 6. Then 300 μl of DPD/FeCl3 were added and the samples were incubated at 37° C. for 20 min, centrifuged for 4 min at max speed and absorbance was measured in OD670 nm. For bacterial H₂S detection, cultures were grown in LB broth for 3 h, then L-cysteine was added at 5 mM final concentration. After 1 h, cultures were centrifuged and 100 μl of supernatant were added to 900 μl of buffer (100 mM potassium phosphate pH 8 and 2.5 mM DTT). Then 200 μl of DPD/FeCl3 were added and the samples were vortexed and incubated for 20 min at 37° C. before absorbance was read at OD670 nm.

### Histology

After sacrifice, kidneys were surgically removed from mice and fixed in 4% paraformaldehyde (PFA), embedded in paraffin, sectioned to 5 μm and subsequently stained with H&E or Masson's trichrome reagents. Histological analysis was performed in a blinded fashion. Abnormal parenchyma was recognized be the presence of one or more of the following: tubular inflammation (tubulitis), tubular dilatation or dropout, interstitial inflammation and or fibrosis. The extent of crystal deposition was also noted. Quantitative scoring was performed as follows: The extent of abnormal (inflamed) renal parenchyma was visually estimated as a percentage of the total cortical area, in well-oriented sections which included both the renal cortex and medulla.

### Host Kidney Gene Expression Analysis

Murine kidney RNA was extracted from paraffin blocks using the RecoverAll™ Total Nucleic Acid Isolation kit (Invitrogen) as per the manufacturer's protocol. Ten 20 μm sections were cut from paraffin blocks using a Leica Jung 2035 Biocut Microtome. Histo-Clear (National Diagnostics) was used as a xylene substitute. To increase digestion efficiency, the Histo-Clear treatment was performed twice, and the digestion time was increased to 30 min at 50° C. Following RNA purification, a secondary DNase treatment was performed using the DNA-free kit (Invitrogen) and following the protocol for rigorous DNase treatment. CDNA was prepared with the iScript cDNA Synthesis Kit (Bio-Rad). RT-PCR analysis was performed using 20 ng of cDNA per 20 μL reaction with KAPA SYBR FAST (Kapa Biosystems). The RT-PCR analysis was performed on an Applied Biosystems Stratagene MX3005P machine. The relative expression of each gene was determined by ^{2-ΔCt} normalized to 13-Actin and GapDH housekeeping genes. List of primers used is provided in Table 3.

### Statistical Analyses

All the statistical analyses were performed using R (v3.4-3.6) on RStudio (v1.25). Mann-Whitney and Kruskal-Wallis were performed as the default statistical tests, unless mentioned otherwise in the figure legends. Sample size for mouse experiments was determined using a sample size calculator.

### REFERENCES—EXAMPLES 1 & 2

Ahlman, B., Leijonmarck, C. E., Lind, C., Vinnars, E., Wernerman, J., 1993. Free amino acids in biopsy specimens from the human colonic mucosa. J. Surg. Res. 55, 647-653. doi.org/10.1006/jsre.1993.1198
Brand, M. W., Wannemuehler, M. J., Phillips, G. J., Proctor, A., Overstreet, A. M., Jergens, A. E., Orcutt, R. P., Fox, J. G., 2015. The altered schaedler flora: Continued applications of a defined murine microbial community. ILAR J. 56, 169-178. doi.org/10.1093/ilar/ilv012
Carbonero, F., Benefiel, A. C., Alizadeh-Ghamsari, A. H., Gaskins, H. R., 2012. Microbial pathways in colonic sulfur metabolism and links with health and disease. Front. Physiol. 3 November, 1-11. doi.org/10.3389/fphys.2012.00448
Castillo-Rodriguez, E., Fernandez-Prado, R., Esteras, R., Perez-Gomez, M., Gracia-Iguacel, C., Fernandez-Fernandez, B., Kanbay, M., Tejedor, A., Lazaro, A., Ruiz-Ortega, M., Gonzalez-Parra, E., Sanz, A., Ortiz, A., Sanchez-Niño, M., 2018. Impact of Altered Intestinal Microbiota on Chronic Kidney Disease Progression. Toxins 10, 300. doi.org/10.3390/toxins10070300
Chen, T. K., Knicely, D. H., Grams, M. E., 2019. Chronic Kidney Disease Diagnosis and Management: A Review. JAMA 322, 1294. doi.org/10.1001/jama.2019.14745
Chen, Y.-Y., Chen, D.-Q., Chen, L., Liu, J.-R., Vaziri, N. D., Guo, Y., Zhao, Y.-Y., 2019. Microbiome—metabolome reveals the contribution of gut—kidney axis on kidney disease. J. Transl. Med. 17, 5. doi.org/10.1186/s12967-018-1756-4
Crews, D. C., Bello, A. K., Saadi, G., 2019. Saudi Journal of Kidney Diseases and Transplantation 10.
Darkoh, C., Plants-Paris, K., Bishoff, D., DuPont, H. L., 2019. Clostridium difficile Modulates the Gut Microbiota by Inducing the Production of Indole, an Interkingdom Signaling and Antimicrobial Molecule. mSystems 4, e00346-18, /msystems/4/2/msys.00346-18.atom.doi.org/10.1128/mSystems.00346-18
David, L. A., Maurice, C. F., Carmody, R. N., Gootenberg, D. B., Button, J. E., Wolfe, B. E., Ling, A. V., Devlin, A. S., Varma, Y., Fischbach, M. A., Biddinger, S. B., Dutton, R. J., Turnbaugh, P. J., 2014. Diet rapidly and reproducibly alters the human gut microbiome. Nature 505, 559-563. doi.org/10.1038/nature12820
Devlin, A. S., Marcobal, A., Dodd, D., Nayfach, S., Plummer, N., Meyer, T., Pollard, K. S., Sonnenburg, J. L., Fischbach, M. A., 2016. Modulation of a Circulating Uremic Solute via Rational Genetic Manipulation of the Gut Microbiota. Cell Host Microbe 20, 709-715. doi.org/10.1016/j.chom.2016.10.021
Fukuuchi, F., Hida, M., Aiba, Y., Koga, Y., Endoh, M., Kurokawa, K., Sakai, H., 2002. Intestinal bacteria-derived putrefactants in chronic renal failure. Clin. Exp. Nephrol. 6, 99-104. doi.org/10.1007/s101570200016
Gao, X. H., Krokowski, D., Guan, B. J., Bederman, I., Majumder, M., Parisien, M., Diatchenko, L., Kabil, O., Willard, B., Banerjee, R., Wang, B., Bebek, G., Evans, C. R., Fox, P. L., Gerson, S. L., Hoppel, C. L., Liu, M., Aryan, P., Hatzoglou, M., 2015. Quantitative H₂S-mediated protein sulfhydration reveals metabolic reprogramming during the integrated stress response. eLife 4, 1-21. doi.org/10.7554/eLife.10067
Hine, C., Mitchell, J., 2017. Endpoint or Kinetic Measurement of Hydrogen Sulfide Production Capacity in Tissue Extracts. Bio-Protoc. 7, 1-18. doi.org/10.21769/BioProtoc.2382
Jia, T., Olauson, H., Lindberg, K., Amin, R., Edvardsson, K., Lindholm, B., Andersson, G., Wernerson, A., Sabbagh, Y., Schiavi, S., Larsson, T. E., 2013. A novel model of adenine-induced tubulointerstitial nephropathy in mice. BMC Nephrol. 14. doi.org/10.1186/1471-2369-14-116
Kogan, A., Gdalevsky, G. Y., Cohen-Luria, R., Goldgur, Y., Phillips, R. S., Parola, A. H., Almog, O., 2009. Conformational changes and loose packing promote E. coli Tryptophanase cold lability. BMC Struct. Biol. 9, 1-12. doi.org/10.1186/1472-6807-9-65
Krishnan, N., Fu, C., Pappin, D. J., Tonks, N. K., 2011. H₂S-Induced Sulfhydration of the Phosphatase PTP1B and Its Role in the Endoplasmic Reticulum Stress Response. Sci. Signal. 4, ra86-ra86. doi.org/10.1126/scisignal.2002329
Kumar, A., Sperandio, V., 2019. Indole Signaling at the Host-Microbiota-Pathogen Interface. mBio 10, e01031-19, /mbio/10/3/mBio.01031-19. doi.org/10.1128/mBio.01031-19
Lee, J., Zhang, X. S., Hegde, M., Bentley, W. E., Jayaraman, A., Wood, T. K., 2008. Indole cell signaling occurs primarily at low temperatures in Escherichia coli. ISME J. 2, 1007-1023. doi.org/10.1038/ismej.2008.54
Linden, D. R., 2014. Hydrogen Sulfide Signaling in the Gastrointestinal Tract. Antioxid. Redox Signal. 20, 818-830. doi.org/10.1089/ars.2013.5312
Liu, Y., Yang, R., Liu, X., Zhou, Y., Qu, C., Kikuiri, T., Wang, S., Zandi, E., Du, J., Ambudkar, I. S., Shi, S., 2014. Hydrogen sulfide maintains mesenchymal stem cell function and bone homeostasis via regulation of Ca2+channel sulfhydration. Cell Stem Cell 15, 66-78. doi.org/10.1016/j.stem.2014.03.005
Lun, H., Yang, W., Zhao, S., Jiang, M., Xu, M., Liu, F., Wang, Y., 2019. Altered gut microbiota and microbial biomarkers associated with chronic kidney disease. MicrobiologyOpen 8, e00678. doi.org/10.1002/mbo3.678
Magee, E. A., Richardson, C. J., Hughes, R., Cummings, J. H., 2000. Contribution of dietary protein to sulfide production in the large intestine: an in vitro and a controlled feeding study in humans. Am J Clin Nutr 72, 1488-1494.
Mishanina, T. V., Libiad, M., Banerjee, R., 2015. Biogenesis of reactive sulfur species for signaling by hydrogen sulfide oxidation pathways. Nat. Chem. Biol. 11, 457-464. doi.org/10.1038/nchembio.1834
Mustafa, A. K., Gadalla, M. M., Sen, N., Kim, S., Mu, W., Gazi, S. K., Barrow, R. K., Yang, G., Wang, R., H, S., 2009. POSTTRANSLATIONAL MODIFICATIONS H 2 S Signals Through Protein S-Sulfhydration. Science 2, 1-9. doi.org/10.1126/scisignal.2000464
Paul, B. D., Snyder, S. H., 2012. H₂S signalling through protein sulfhydration and beyond. Nat. Rev. Mol. Cell Biol. 13, 499-507. doi.org/10.1038/nrm3391
Peng, H., Zhang, Y., Palmer, L. D., Kehl-Fie, T. E., Skaar, E. P., Trinidad, J. C., Giedroc, D. P., 2017. Hydrogen Sulfide and Reactive Sulfur Species Impact Proteome S-Sulfhydration and Global Virulence Regulation in Staphylococcus aureus. ACS Infect. Dis. 3, 744-755. doi.org/10.1021/acsinfecdis.7b00090
Phillips, R. S., Gollnick, P. D., 1989. Evidence that cysteine 298 is in the active site of tryptophan indole-lyase. J. Biol. Chem. 264, 10627-10632.
Poesen, R., Mutsaers, H. A. M., Windey, K., van den Broek, P. H., Verweij, V., Augustijns, P., Kuypers, D., Jansen, J., Evenepoel, P., Verbeke, K., Meijers, B., Masereeuw, R., 2015. The Influence of Dietary Protein Intake on Mammalian Tryptophan and Phenolic Metabolites. PLOS ONE 10, e0140820. doi.org/10.1371/journal.pone.0140820
Ren, J., Sang, Y., Lu, J., Yao, Y. F., 2017. Protein Acetylation and Its Role in Bacterial Virulence. Trends Microbiol. 25, 768-779. doi.org/10.1016/j.tim.2017.04.001
Rosshart, S. P., Herz, J., Vassallo, B. G., Hunter, A., Wall, M. K., Badger, J. H., McCulloch, J. A., Anastasakis, D. G., Sarshad, A. A., Leonardi, I., Collins, N., Blatter, J. A., Han, S.-J., Tamoutounour, S., Potapova, S., 2019. Laboratory mice born to wild mice have natural microbiota and model human immune responses 14.
Schulz, M. D., Atay, Ç., Heringer, J., Romrig, F. K., Schwitalla, S., Aydin, B., Ziegler, P. K., Varga, J., Reindl, W., Pommerenke, C., Salinas-Riester, G., Bock, A., Alpert, C., Blaut, M., Polson, S. C., Brandi, L., Kirchner, T., Greten, F. R., Polson, S. W., Arkan, M. C., 2014. High-fat-diet-mediated dysbiosis promotes intestinal carcinogenesis independently of obesity. Nature 514, 508-512. doi.org/10.1038/nature13398
Sen, N., Paul, B. D., Gadalla, M. M., Mustafa, A. K., Sen, T., Xu, R., Kim, S., Snyder, S. H., 2012. Hydrogen sulfide-linked sulfhydration of NF-κB mediates its antiapoptotic actions. Mol. Cell 45, 13-24. doi.org/10.1016/j.molcel.2011.10.021
Shimada, T., Tanaka, K., Ishihama, A., 2016. Transcription factor DecR (YbaO) controls detoxification of L-cysteine in Escherichia coli. Microbiol. U. K. 162, 1698-1707. doi.org/10.1099/mic.0.000337
Shimizu, T., Masuda, S., 2019. Persulphide-responsive transcriptional regulation and metabolism in bacteria. J. Biochem. (Tokyo) mvz063. doi.org/10.1093/jb/mvz063
Thielemann, L. E., Oberhauser, E. W., Rosenblut, G., Videla, L. A., Valenzuela, A., 1990. Sulfur-containing amino acids that increase renal glutathione protect the kidney against papillary necrosis induced by 2-bromoethylamine. Cell Biochem. Funct. 8, 19-24. doi.org/10.1002/cbf.290080104
Tokushige, M., Tsujimoto, N., Oda, T., Honda, T., Yumoto, N., Ito, S., Yamamoto, M., Kim, E. H., Hiragi, Y., 1989. Role of cysteine residues in tryptophanase for monovalent cation-induced activation. Biochimie 71, 711-720. doi.org/10.1016/0300-9084 (89)90087-4
Vaziri, N. D., Wong, J., Pahl, M., Piceno, Y. M., Yuan, J., DeSantis, T. Z., Ni, Z., Nguyen, T.-H., Andersen, G. L., 2013. Chronic kidney disease alters intestinal microbial flora. Kidney Int. 83, 308-315. doi.org/10.1038/ki.2012.345
Whitt, D. D., Demoss, R. D., 1975. Effect of microflora on the free amino acid distribution in various regions of the mouse gastrointestinal tract. Appl. Microbiol. 30, 609-15.
Wlodarska, M., Luo, C., Kolde, R., D'Hennezel, E., Annand, J. W., Heim, C. E., Krastel, P., Schmitt, E. K., Omar, A. S., Creasey, E. A., Garner, A. L., Mohammadi, S., O'Connell, D. J., Abubucker, S., Arthur, T. D., Franzosa, E. A., Huttenhower, C., Murphy, L. O., Haiser, H. J., Vlamakis, H., Porter, J. A., Xavier, R. J., 2017. Indoleacrylic Acid Produced by Commensal Peptostreptococcus Species Suppresses Inflammation. Cell Host Microbe 22, 25-37.e6. doi.org/10.1016/j.chom.2017.06.007
Xu, K.-Y., Xia, G.-H., Lu, J.-Q., Chen, M.-X., Zhen, X., Wang, S., You, C., Nie, J., Zhou, H.-W., Yin, J., 2017. Impaired renal function and dysbiosis of gut microbiota contribute to increased trimethylamine-N-oxide in chronic kidney disease patients. Sci. Rep. 7, 1445. doi.org/10.1038/s41598-017-01387-y
Zelante, T., Iannitti, R. G., Cunha, C., DeLuca, A., Giovannini, G., Pieraccini, G., Zecchi, R., D'Angelo, C., Massi-Benedetti, C., Fallarino, F., Carvalho, A., Puccetti, P., Romani, L., 2013. Tryptophan catabolites from microbiota engage aryl hydrocarbon receptor and balance mucosal reactivity via interleukin-22. Immunity 39, 372-385. doi.org/10.1016/j.immuni.2013.08.003
Zhao, L., Zhang, F., Ding, X., Wu, G., Lam, Y. Y., Wang, Xuejiao, Fu, H., Xue, X., Lu, C., Ma, J., Yu, L., Xu, C., Ren, Z., Xu, Y., Xu, S., Shen, H., Zhu, X., Shi, Y., Shen, Q., Dong, W., Liu, R., Ling, Y., Zeng, Y., Wang, Xingpeng, Zhang, Q., Wang, J., Wang, L., Wu, Y., Zeng, B., Wei, H., Zhang, M., Peng, Y., Zhang, C., 2018. Gut bacteria selectively promoted by dietary fibers alleviate type 2 diabetes. Science 359, 1151-1156. doi.org/10.1126/science.aao5774
Zou, J., Chassaing, B., Singh, V., Pellizzon, M., Ricci, M., Fythe, M. D., Kumar, M. V., Gewirtz, A. T., 2017. Fiber-Mediated Nourishment of Gut Microbiota Protects against Diet-Induced Obesity by Restoring IL-22-Mediated Colonic Health. Cell Host Microbe 23, 41-53.e4. doi.org/10.1016/j.chom.2017.11.003

1. A method of regulating the level or activity of hydrogen sulfide (H₂S) in the gastrointestinal tract of a subject, the method comprising: administering to the subject a composition comprising a sulfated amino acid.

2. The method of claim 1, wherein the composition comprises one or more of a sulfated amino acid selected from the group consisting of: methionine, cysteine, homocysteine, taurine, cystine (di-cysteine), salts, analogs, and derivatives thereof.

3. The method of claim 1, wherein the composition comprises at least one food ingredient.

4. The method of claim 3, wherein the food ingredient is selected from the group consisting of: fats, carbohydrates, proteins, fibers, nutritional balancing agents, and mixtures thereof.

5. The method of claim 1, wherein the composition is formulated as a dietary supplement.

6. The method of claim 1, wherein the composition is formulated as a medical food.

7. The method of claim 1, wherein the composition is formulated as a pharmaceutical composition.

8. The method of claim 1, wherein the administering is oral administration, enteral administration, or parenteral administration.

9. The method of claim 1, wherein the subject is a mammal.

10. The method of claim 1, wherein the subject is a human, a dog, or a cat.

11. The method of claim 1, wherein the subject has or is suspected of having an inflammatory or fibrotic disease of the kidney.

12. A method of treating an inflammatory or fibrotic disease of the kidney in a subject, the method comprising: administering to a subject in need thereof a composition comprising a sulfated amino acid.

13. The method of claim 12, wherein the composition comprises one or more of a sulfated amino acid selected from the group consisting of: methionine, cysteine, homocysteine, taurine, cystine (di-cysteine), salts, analogs, and derivatives thereof.

14. The method of claim 12, wherein the composition is formulated as a dietary supplement.

15. The method of claim 12, wherein the composition is formulated as a medical food.

16. The method of claim 12, wherein the composition is formulated as a pharmaceutical composition.

17. The method of claim 12, wherein the administering is oral administration, enteral administration, or parenteral administration.

18. The method of claim 12, wherein the subject is a mammal.

19. The method of claim 12, wherein the subject is a human, a dog, or a cat.

20. The method of claim 12, wherein the inflammatory or fibrotic disease of the kidney is selected from the group consisting of: chronic kidney disease, renal parenchymal injury, tubulitis, end-stage renal failure, lupus, nephritis, acute renal failure, kidney infection, polycystic kidney disease, renal amyloidosis, and renal colic.


SEQUENCE LISTING (26 sequences)

SEQ ID NO: 1 - DNA, 30 bp, Artificial Sequence
  Description of Artificial Sequence Synthetic primer
atatggatcctctggcggtaggtctgtatg

SEQ ID NO: 2 - DNA, 46 bp, Artificial Sequence
  Description of Artificial Sequence Synthetic primer
gtggtggtggtggtggtgctcgagaacttctttaagttttgcggtg

SEQ ID NO: 3 - DNA, 52 bp, Artificial Sequence
  Description of Artificial Sequence Synthetic primer
ctcgagcaccaccaccaccaccactaattaatactacagagtggctataagg

SEQ ID NO: 4 - DNA, 33 bp, Artificial Sequence
  Description of Artificial Sequence Synthetic primer
atatgcggccgcccaaacacgatcacaaaggag

SEQ ID NO: 5 - DNA, 31 bp, Artificial Sequence
  Description of Artificial Sequence Synthetic primer
atatggatccgcgtatttgattaccggcaac

SEQ ID NO: 6 - DNA, 51 bp, Artificial Sequence
  Description of Artificial Sequence Synthetic primer
gtcctgacctgattctggatatttattctaacatagcccttccacagagaa

SEQ ID NO: 7 - DNA, 51 bp, Artificial Sequence
  Description of Artificial Sequence Synthetic primer
ttctctgtggaagggctatgttagaataaatatccagaatcaggtcaggac

SEQ ID NO: 8 - DNA, 32 bp, Artificial Sequence
  Description of Artificial Sequence Synthetic primer
atatgcggccgctgcaccaccatccaactacc

SEQ ID NO: 9 - protein, 21 aa, Unknown
  Description of Unknown TnaA sequence
LLPHIPADQFPAQALACELYK

SEQ ID NO: 10 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 11 - protein, 460 aa, Pseudoflavonifractor sp.
MLDFTKYPAEPFRIKSVETVKMNTRAEREEIAKAAGYNTFLINSEDVYIDLLTDSGTNAMSDKQWGGLMQGDEAYAGSRNWFHLEATVQELFGFKHVIPTHQGRGAENILGHIAIKPGQYVPGNMYFTTTRFHQEANGGIFRDIIIDEAHDATLDLPFKGNVDIKKVQALIDEVGAENIAYLCVAVTVNLAGGQPVSMQNLREVSEICHKNKIRMFMDATRCVENAYFIKEREEGYADVAIKDIVHEMFSYADGCTMSGKKDCLTNIGGFLCMNDDDLYMNAREWVVVYEGMPSYGGMAGRDMEAMAIGLRESMQEEYIEHRIKQVRYLGDRLLEAGVPIIEPVGGHAVFLDARRFCPHLTQDQLPAQALAAQLYIDSGVRSMERGIVSAGRDKDTGEDHHPKLETVRLTIPRRVYTYAHMDFIADAVIDLYKNKEEIRGLKWVYEPKMLRFFTGRFEHI

SEQ ID NO: 12 - protein, 449 aa, Alistipes putredinis
ESWKIKMVEPIKKSTREEREQWLKEAKYNVFMLRSDQVYIDCITDSGTGAMSDRQWAAMMTGDESYAGARSFYELKDTITRITGFEYVIPTHQGRAAENVLFSHLVKEGDIVPGNSHFDTTKGHIESRKAFAVDCTVDEAKDTQLEVPFKGNVDLDKLEKCLAENADKIPFIIVTITNNTAGGQPVSMDNLRGVRKIADKYNKRVVFDSARFAENAYFIKMRDPQYKDATIKEICKEMFSLADIMTMSSKKDGLVNMGGFIATRWEDIYEGAKRFVIPFEGFLTYGGMNGRDMAALSVGLDENTEFDMLQTRIKQVEYLAAKLDEYGIPYQRPVGGHALFIDADKVLDRIPKEEFPAQRLTMELYLEAGVRGCEIGYLLADRDPITRENRFTGNDFLRLCICRRTYTNNHMDVIAAALKNVYDRRNEITRGVKITWEAELMRHFTVQLE

SEQ ID NO: 13 - protein, 448 aa, Bacteroides sp.
ESWKIKMVEPIRKSTREEREQWLKECDYNVFMLKSEQVYIDCITDSGTGAMSDRQWAAMMMGDESYAGARSFFNLKETITRITGFEYVLPTHQGRAAENVLFSYLVHEGDIIPGNSHFDTTKGHIESRKAFAVDCTIDEAKDTQLEHPFKGNVDINKLEKVLKEQGDKVPFVIVTITNNTAGGQPVSMQNLREVNVLCEKYKKPLIFDSARFAENAYFIKTREEGYADKTIKEIAREMFDLADGMTMSSKKDGLVNMGGFIATRHKDWYEGAKGFCICFEGFVTYGGMNGRDMDALAVGLDENTEFDMLETRIRQVQYLASKLDEYGIPYQRPVGGHAIFLDADKILTEVPKEEFPAQMLTVELYREAGIRGCEIGYILADRDPVTRENRFGGLDFLRLCIARRVYTNNHMDVIAAAIKNVYDRRHEVKRGLKIVWEAPLLRHFTVKL

SEQ ID NO: 14 - protein, 449 aa, Bacteroides intestinalis
ESWKIKMVEPIRKSTRQEREQWLKEAHYNVFQLKSEQVYIDLITDSGTGAMSDRQWAAMMLGDESYAGASSFFNLKEVITRLTGFEYVIPTHQGRAAENVLFSYLVHEGDIVPGNSHFDTTKGHIEGRKAIALDCTIDEAKNTQLEIPFKGNVDPVKLEKALQEHADRIPFIIVTITNNTAGGQPVSMQNLREVRALADKYHKPVLFDSARFAENAYFIKMREDGYSGKTIKEITKEMFDLADGMTMSAKKDGIVNMGGFIATRKKDWYEGAKGFCVQYEGYLTYGGMNGRDMNALATGLDENTEFDNLETRIRQVEYLAKKLDEYGIPYQRPAGGHAIFVDATKVLTHVPKNEFPAQTLTVELYLEAGIRGCEIGYILADRDPVTRENRFTGLDLLRLAIPRRVYTDNHMNVIAVALKNVFDRRESITNGVRITWEAPLMRHFTVQLE

SEQ ID NO: 15 - protein, 451 aa, Bacteroides thetaiotaomicron
ESWKIKMVEPIRKSTREEREQWIKEAHYNVFQLKSEQVYIDLITDSGTGAMSDRQWAGMMLGDESYAGATSFFKLKDTITRITGFEYIIPTHQGRAAENVLFSYLVHEGNIVPGNSHFDTTKGHIEGRHAIALDCTIDEAKNTQLEIPFKGNVDPAKLEKALSEYADRIPFIIVTITNNTAGGQPVSMQNLREVRAIADKYNKPVLFDSARFAENAYFIKMREEGYQDKSIKEITREMFDLADGMTMSAKKDGIVNMGGFIATRRKEWYEGAKGFCVQYEGYLTYGGMNGRDMNALAIGLDENTEFDNLETRIKQVEYLAQKLDEYEIPYQRPAGGHAIFVDASKVLTHVPKEEFPAQTLTVELYLEAGIRGCEIGYILADRDPITHENRFNGLDLLRLAIPRRVYTDNHMNVIAAALRNVFERRETITRGVRIAWEAPLMRHFTVQLERL

SEQ ID NO: 16 - protein, 451 aa, Bacteroides acidifaciens
ESWKIKMVEPIRKSTREEREQWIKEAHYNVFQLKSEQVYIDLITDSGTGAMSDRQWAGMMLGDESYAGATSFFKLKEMITKLTGFEYIIPTHQGRAAENVLFSYLVHEGDIVPGNSHFDTTKGHIEGRRATALDCTIDAAKQTQLELPFKGNVDPDKLQKALTEHAERIPFIIVTITNNTAGGQPVSMQNLREVRAIADKYGKPVLFDSARFAENAYFIKMHEEGYQDKSIKEITKEMFALADGMTMSAKKDGIVNMGGFIATRREDWYEGAKGFCVQYEGYLTYGGMNGRDMNALAIGLDENTEFDNLETRIKQVEYLAKKLDEYGIPYQRPAGGHAIFIDAPKVLTHVPKEEFPAQTLTVELYLEAGIRGCEIGYILADRDPVTHENRFNGLDLLRLAIPRRVYTDNHMNVIAAALKNVYERRESITRGVRIAWEAPLMRHFTVQLERL

SEQ ID NO: 17 - protein, 451 aa, Bacteroides uniformis
EAWKIKMVEPIRKSTRQEREQWLKEAHYNVFQLKSEQVYIDLITDSGTGAMSDRQWAGMMLGDESYAGATSFFKLKEVITRLTGFEYIIPTHQGRAAENVLFSYLVHEGDIVPGNSHFDTTKGHIEGRRALALDCTIDEAKQTQLEIPFKGNVDPKKLEKALQEHSERIPFVIVTITNNTAGGQPVSMQNLREVRAIADKYGKPVLFDSARFAENAYFIKMREEGYRNKSIKEITREMFDLADGMTMSAKKDGIVNMGGFIATRRKDWYEGAKGFCVQFEGYLTYGGMNGRDMNALAIGLDENTEFDNLETRIHQVGYLAMKLDEYGIPYQRPAGGHAIFVDAPKVLTHVPKEEFPAQTLTAELYLEAGIRGCEIGYLLADRDPVTRENRFNGLDLLRLAIPRRVYTDNHMDVVAAALKNVYDKRESITRGVRITWEAPLMRHFTVQLERL

SEQ ID NO: 18 - protein, 451 aa, Bacteroides nordii
ESWKIKMVEPIRKSTRQEREQWIKEAHYNVFQLKAEQVYIDLITDSGTGAMSDRQWAGMMLGDESYAGASSFFKLKDKITQLTGFEYIIPTHQGRAAENVLFSYLVHEGDIVPGNSHFDTTKGHIEGRKALALDCTIDEAKQTQLEIPFKGNVDPAKLEKALQEHADRIPFIIVTITNNTAGGQPVSMQNLREVRALADKYNKPVLFDSARFAENAYFIKTREEGYGNKTIKKITREMFDLADGMTMSAKKDGIVNMGGFIATRRKEWYEGAKGFCVQYEGYLTYGGMNGRDMNALAIGLDENTEFDNLETRIHQVQYLAKKLDEYGIPYQRPAGGHAIFVDAPKVLTCVPKEEFPAQTLTIELYLEAGIRGCEIGYLLADRDPVTRQNRFNGLDLLRLAIPRRVYTDNHMDVIAAALKNVYDRRENVTRGVRIAWEAPLMRHFTVQLERL

SEQ ID NO: 19 - protein, 448 aa, Bacteroides clarus
ESWKIKMVEPIRKSTREEREQWLRDAHYNVFQLRADQVYIDLLTDSGTGAMSDRQWGAMMTGDESYAGARSFFRLKETVGRLTGFDYFIPTHQGRAAENVLFSHLVKEGDIVPGNSHFDTTKGHIESRKAFALDCTVDEARDTQLEVPFKGNVDIAKLEKALEEYHDRVPFIIVTITNNTAGGQPVSLQNLREVRAVADRWGKRVILDSARFAENAYFIKTREEGQGARTIKEIVLDMFSLADAMTMSAKKDGIVNMGGFIATRHEDWYEGAKRFCIPFEGYLTYGGMNGRDMEALAVGLDENTEFDNLHSRIHQVEYLAKRLDEFGIPYQRPAGGHAIFVDASKVLTHVPREEFPAQTLTVELYREAGIRGCEIGYMLADRDPVTRENRFGGLDLLRLAIPRRVYTDNHMDVIAVALKNVYDRRESITRGLRIIWEAELMRHFTVKL

SEQ ID NO: 20 - protein, 448 aa, Prevotella sp.
ESWRIKMVEPIRKSTREEREQWIKAAHYNVFQLPAEQVYIDLLTDSGTGAMSDRQWAAMMTGDESYAGARSFFNMKNAVERITGFPIVIPTHQGRAAENVLFSALVKEGDIVPGNSHFDTTKGHIESRKAFAVDCTIDEAKDTQLEHPFKGNVDIDKLEKAVSENPGRVPFIIVTITNNTAGGQPVSMANLKAVRELADRHNLRVIFDSARFAENAYFIKMREEGYADKSIKEIVREMFALADGMTMSAKKDAIVNMGGFFATRHEDWADGARRFAIPFEGYLTYGGMNGRDMEALAVGLDENTEFDNLHTRIHQVEYLAKRLDEFGIPYQRPAGGHAIFVDASKILTNVPKEEFPAQTLTVELYREAGIRGCEIGYILADRDPVTRENRFGGLDLLRLAIPRRTYTDNHMDVVAVALKNVYDRRNEITRGVKITWEAPLMRHFTVQL

SEQ ID NO: 21 - protein, 448 aa, Bacteroides sp.
ESWRIKMVEPIRKSTRQEREQWIREAHYNVFQLPAEQVYIDLLTDSGTGAMSDRQWSAMMMGDESYAGARSFFNMRDTIERITGFEHVIPTHQGRAAENVLFSALVKKGDVVPGNSHFDTTKGHIESRGAFAVDCTIDEARDTQLEHPFKGNVDIDKLEKAVTEAAHVPFIIVTVTNNTAGGQPVSMANLRAVRELADRHGLRVIFDSARFAENAYFIKTREEGYADKTIKEIVREMFSLADGMTMSAKKDAIVNMGGFIATRHADWAEAAKCFSIPFEGYLTYGGMNGRDMEALAVGLDENTEFDNLHSRIHQVEYLAKRLDEFGIPYQRPAGGHAIFVDASKILTEIPKEEFPAQTLTVELYLEAGIRGCEIGYILADRDPVTRENRFGGLDLLRLAIPRRTYTDNHMDVVAVALKNVFDRRHEITRGVRITWEAPLMRHFTVQLE

SEQ ID NO: 22 - protein, 452 aa, Odoribacter splanchnicus
ESFKIKMVETIRKSTREEREQWIKEAKYNLFNLKSDDVFIDLLTDSGTGAMSDRQWAEMMLGDESYAGARSYYKMKEAIKNILGFDYFLPTHQGRAAENVLYSTLIKEGDVLPGNSHFDTTKGHIEFRKALAIDCTIDEAADTQLEIPFKGNMDPAKLEAVLKKYPKEKIPAVVLTVTNNTAGGQPVSMQNIREVSALCRKYGVNLQIDSARFAENAYFIKTREKGYENKSIKEIVKEMFSYADIMTMSSKKDAIVNMGGFVAFKNEELWKKCQMFCIMNEGFITYGGMSGRDMNALAQGLDEGTEFDYLETRIRQVEYLGVRLDEYGIPYQRPAGGHAIFVDAKKILTHVPKEEFIAQTLGVELYLEAGIRGVEIGSILADRDPVTRENRYPKLELLRLAIPRRTYTNNHMDVIAVALKNVYDRREQITHGYRITKEFPIMRHFTVELEPV

SEQ ID NO: 23 - protein, 451 aa, Unknown
  Description of Unknown Culturomica massiliensis sequence
ESYRIKMVETIRKSTREEREQWIKEAKYNLFALKSDHVFIDLLTDSGTGAMSDKQWSAMMLGDESYAGARSYYNMKNAIKDILGFDYFLPTHQGRAAENVLYSTIIKEGDILPGNSHFDTTKGHIEFRKAVALDCTIDEAADTQLEIPFKGNLDPAKLEEVLKKYPKEKIPAVVLTVTNNTAGGQPVSMQNIREVSALCKKYGVLLQMDSARFAENAYFIKVREKGYENKTIKEIVKEMFSYADIMTMSSKKDAIVNMGGFVAFKNEELWKRCQMFCIMNEGFITYGGMSGRDMNALAQGLDEGTEFDYLETRIGQVEYLGAKLDEYGIPYQRPAGGHAIFVDAKKILTHVPKEEFIAQTLGVELYLEAGIRGVEIGSILADRDPVTRENRYPRLELLRLAIPRRTYTNNHMDVIAAALKNVYDRRESITRGYVITKEFPIMRHFTVELEK

SEQ ID NO: 24 - protein, 452 aa, Alistipes sp.
EPYKIKTTEAIRTSTREEREQWLAEARYNLFNLRSDQVFIDLLTDSGTGSMSDRQWAGMMTGDESYAGASSYYRLKETIERIFGMPYFLPTHQGRAAENVIFSALLKEGDVVPGNSHFDTTKGHIEFRHCSAPDCTIDAAADTQLEIPFKGEMDLEKLERVFKSHPKEKIPCTVLTITNNTAGGQPVSMKNIRETAELCRRYGIPLLIDSARFAENAYFIKTREAGYADKTIKEIVREIYQYADIMTISAKKDGVVNMGGFVAMRSEELRRKAMTFGIMYEGYVTYGGMSGRDMDALAVGLDENTEFEQLDARIRQVKLLGDLLDEYGVPYQRPAGGHAIFIDAKKILPNLPKEQFIAQTLGCELYLEAGIRGVEIGSILADRDPDTHENRYPRLELLRLAIPRRVYTDNHIRVIAAACRNIYERRDEITKGYRITFEAPILRHFTVELEPV

SEQ ID NO: 25 - protein, 450 aa, Alistipes sp.
EPYKIKMTEAIRPSTRAEREQWIREAKYNLFKLRSDQVTIDLLTDSGTGSMSDRQWAAMMTGDESYAGASSYFRLKQIVNDLFGMPHFLPTHQGRAAENVIFSALLKEGDIVPGNSHFDTTKGHIEFRRCHAIDCTIDAAADTQKELPFKGEMDLEKLEHVLKSHPKEKIPCVVLTITNNTAGGQPVSMRNIRETAELCRRYGIPMLIDSARFAENAYFIKTREAGYENKSIKEIVREIYTYADLMTISAKKDGVVNMGGFLAMHDEELYRKAMQYSIMYEGYLTYGGMSGRDMDALAVGLDENTEFEQLDARIRQVKLLGDLLDEYGVPYQRPAGGHAIFVDAKKILPNLPKEEFIAQTLGIELYLEAGIRGVEIGSILADRDPETHENRYPKLELLRLAIPRRVYTDNHIRVIAAACRNIYERREQITSGYKITFETPILRHFTVELE

SEQ ID NO: 26 - protein, 479 aa, Artificial Sequence
  Description of Artificial Sequence Synthetic polypeptide
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEVLEHHHHHH